(19) United States
(12) Patent Application Publication
(10) Pub. No.: US 2024/0132448 A1
(43) Pub. Date: Apr. 25, 2024

(54) ORGANIC COMPOUNDS AND USES THEREOF

(71) Applicant: Zhejiang Brilliant Optoelectronic Technology Co.,Ltd., Taizhou (CN)

(72) Inventors: Junyou PAN, Taizhou (CN); Huaijun CHEN, Taizhou (CN); Jiahui TAN, Taizhou (CN); Klaus MULLEN, Taizhou (CN)

(73) Assignee: Zhejiang Brilliant Optoelectronic Technology Co.,Ltd., Taizhou (CN)

(21) Appl. No.: 18/518,743

(22) Filed: Nov. 24, 2023

Related U.S. Application Data
(63) Continuation of application No. PCT/CN2022/094943, filed on May 25, 2022.

(30) Foreign Application Priority Data
May 25, 2021 (CN) 202110573058.5

(51) Int. Cl. C07D 209/86; C07B 59/00; C07C 15/38; C07C 25/13; C07D 213/24; C07D 273/01; C07D 307/91; C07D 311/02; C07D 333/76; C07D 407/10; C07D 493/04; H10K 85/20; H10K 85/60

(52) U.S. Cl. CPC C07D 209/86; C07B 59/004; C07C 15/38; C07C 25/13; C07D 213/24; C07D 273/01; C07D 307/91; C07D 311/02; C07D 333/76; C07D 407/10; C07D 493/04; H10K 85/20; H10K 85/622; H10K 85/654; H10K 85/6572; H10K 85/6574; H10K 85/6576; C07B 2200/05

(57) ABSTRACT

Disclosed are organic compounds including a structure of formula (I). The organic compound has a good rigid structure, so that the organic compound has good thermal stability, and high efficiency and the long lifetime of an OLED device are realized. Also provided are formulations containing the organic compounds, and at least one organic solvent. Further provided are organic electronic devices containing a functional layer, the functional layer comprises the organic compounds.

### CROSS-REFERENCE TO RELATED APPLICATIONS

The present application is a continuation of International Application No. PCT/CN2022/094943, filed on May 25, 2022, which claims priority to Chinese Patent Application No. 202110573058.5, filed on May 25, 2021. All of the aforementioned applications are incorporated herein by reference in their entireties.

### TECHNICAL FIELD

The present disclosure relates to the field of organic photoelectric material and device technology, and in particular to an organic compound, a formulation, and the applications thereof in organic electronic devices, particularly in organic electroluminescent devices. The present disclosure also relates to an organic electronic device comprising an organic compound as described herein, and the applications thereof.

### BACKGROUND

Due to the diversity of the structure and the synthesis, low manufacturing cost, and excellent optical and electrical properties, the organic semiconducting materials show great potential in the applications. In particular, as the most promising next-generation display technology, the commercialization of organic light-emitting diodes (OLEDs) is being developed.

Up to now, a variety of emitting material systems based on fluorescent and phosphorescent materials have been developed so far. OLEDs using fluorescent materials present high reliability, but their internal quantum efficiency for electroluminescence is limited to 25% due to the 1:3 branching ratio between the singlet and triplet excited states of the excitons. At present, the device lifetime based on blue phosphorescent material system still cannot meet the practical requirements. Therefore, the blue fluorescent material system is still a current research hotspot in both industry and academic research.

The performance of the light-emitting layer material usually determines the efficiency and lifetime of the light-emitting device, in which the host material undertakes the transport of the electrons and holes, recombination, and exciton energy transfer, so the properties of the host material have a very important impact on the performance of the entire device, especially the structural stability of the host material in the excited state directly determines the lifetime of the device. The obvious TTA (triplet-triplet annihilation) effect of the anthracene compounds in the current blue fluorescent material system improves the luminous efficiency of the fluorescent system. However, as the host materials, the anthracene compounds are easily damaged in the excited state, thus reducing the lifetime of the device. At present, the lifetime of blue materials has long been the short board of the OLED technology.

Therefore, the existing technologies, especially the material solutions, still need to be improved and developed.

### SUMMARY

In one aspect, the present disclosure provides an organic compound comprising a structure of formula (I):

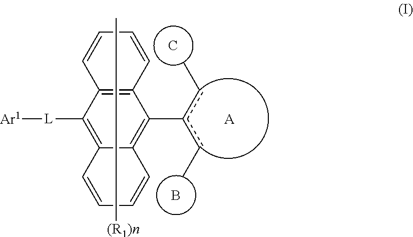

Where A, B, C are the same or different, and are independently selected from the group consisting of a substituted/unsubstituted fused aromatic or heteroaromatic ring structure containing 5 to 30 ring atoms, B and C are each linked to A at the adjacent position of the linkage site between A and anthracene unit, and B and C may be fused with A to form a ring, respectively; Ar¹ is selected from a substituted/unsubstituted fused aromatic or heteroaromatic ring structure containing 8 to 40 ring atoms; L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof; R₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ may be bridged to a carbon atom on an adjacent or linked aromatic ring via a bridging group, the bridging group being the same or different as a single bond, or a di-bridging group, or tri-bridging group; n is integer from 0 to 8, and the adjacent two substituents can be fused to form a ring.

In another aspect, the present disclosure also provides a polymer comprising at least one repeating unit, the at least one repeating unit comprises a structure of formula (I).

In yet another aspect, the present disclosure further provides a mixture comprising an organic compound or a polymer as described above, and at least one organic functional material, the at least one organic functional material is selected from a hole-injection material (HIM), a hole-transport material (HTM), an electron-transport material (ETM), an electron-injection material (EIM), an electron-blocking material (EBM), a hole-blocking material (HBM), an emitting material (Emitter), or a host material (Host).

In yet another aspect, the present disclosure further provides a formulation comprising an organic compound or a polymer as described above, and at least one organic solvent.

In yet another aspect, the present disclosure further provides an organic electronic device comprising a functional layer, the functional layer comprises an organic compound or a polymer or a mixture as described above.

Beneficial effect: introducing the bulky group onto benzene or other aromatic compounds, which are linked with anthracene and its derivatives at 9- and 10-position of anthracene, at the adjacent position of the linkage site between above-mentioned benzen or other aromatic compounds and anthracene and its derivatives could adjust the energy level of the materials and improve their photo-oxidation stability as well as their stability in the excited state, thus enhancing the efficiency and lifetime of the devices based on the organic compound as described herein. The luminous efficiency and the lifetime of the electroluminescent device can be improved by using the organic compound as described herein as a host material and other suitable materials. Therefore, the present disclosure provides a solution for manufacturing a light-emitting device with high-efficiency and long-lifetime.

### DETAILED DESCRIPTION OF THE EMBODIMENTS

The present disclosure is to provide an organic compound, a formulation, an organic electronic device, and the applications thereof, particularly in the organic electroluminescent devices, aiming to improve the efficiency and lifetime of a blue host material.

In order to make the objects, technical solutions, and effects of the present disclosure more clear and definite, the present disclosure is further described in detail below. It should be understood that the embodiments described herein are only intended to explain the present disclosure and are not intended to limit the present disclosure.

As used herein, the terms “formulation”, “printing ink”, and “ink” have the same meaning, and they are interchangeable with each other.

As used herein, the terms “host material”, “matrix material” have the same meaning, and they are interchangeable with each other.

As used herein, the term “substituted” means that a hydrogen atom of the compound is substituted.

As used herein, “the number of ring atoms” means that the number of atoms constituting the ring itself of a structural compound (e. g., a monocyclic compound, a fused ring compound, a cross-linked compound, a carbocyclic compound, and a heterocyclic compound) by covalent bonding. When the ring is substituted with a substituent, the atoms contained in the substituent are not included in the ring atoms. The above rule applies for all cases without further specific description. For example, the number of ring atoms of a benzene ring is 6, the number of ring atoms of a naphthalene ring is 10, and the number of ring atoms of a carbazole group is 13.

In embodiments of the present disclosure, the energy level structure of the organic material, singlet energy level (E_{S1}), triplet energy level (E_{T1}), highest occupied molecular orbital (HOMO), and lowest unoccupied molecular orbital (LUMO) play key roles. The determination of these energy levels is introduced as follows.

HOMO and LUMO energy levels can be measured by photoelectric effect, for example by XPS (X-ray photoelectron spectroscopy), UPS (UV photoelectron spectroscopy), or by cyclic voltammetry (hereinafter referred to as CV). Recently, quantum chemical methods, such as density functional theory (hereinafter referred to as DFT) are becoming effective method for calculating the molecular orbital energy levels.

The singlet energy level E_{S1} of the organic material can be determined by the emission spectrum, and the triplet energy level E_{T1} of the organic material can be measured by low-temperature time-resolved spectroscopy. E_{S1} and E_{T1} can also be calculated by quantum simulation (for example, by Time-dependent DFT), for instance with the commercial software Gaussian 09W (Gaussian Inc.), the specific simulation method can be found in WO2011141110 or as described in the following embodiments. ΔE_{ST} stands for E_{S1}-E_{T1}.

It should be noted that the absolute values of HOMO, LUMO, E_{S1} and E_{T1} may vary depending on the measurement method or calculation method used. Even for the same method, different ways of evaluation, for example, using either the onset or peak value of a CV curve as reference, may result in different (HOMO/LUMO) values. Therefore, reasonable and meaningful comparison should be carried out by using the same measurement and evaluation methods. In the embodiments of the present disclosure, the values of HOMO, LUMO, E_{S1} and E_{T1} are based on the Time-dependent DFT simulation, which however should not exclude the applications of other measurement or calculation methods.

In the present disclosure, (HOMO−1) stands for the energy level of the second highest occupied molecular orbital, (HOMO−2) stands for the energy level of the third highest occupied molecular orbital, and so on. (LUMO+1) stands for the energy level of the second lowest unoccupied molecular orbital, (LUMO+2) stands for the energy level of the third lowest occupied molecular orbital, and so on.

In one aspect, the present disclosure provides an organic compound comprising a structure of formula (I):

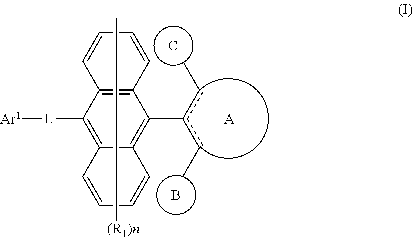

Where A, B, C are the same or different, and are independently selected from the group consisting of a substituted/unsubstituted fused aromatic or heteroaromatic ring structure containing 5 to 30 ring atoms, B and C are each linked to A at the adjacent position of the linkage site between A and anthracene unit, and B and C may be fused with A to form a ring, respectively; Ar¹ is selected from a substituted/unsubstituted fused aromatic or heteroaromatic ring structure containing 8 to 40 ring atoms; L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof; R₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ may be bridged to a carbon atom on an adjacent or linked aromatic ring via a bridging group, the bridging group being the same or different as a single bond, or a di-bridging group, or tri-bridging group; n is integer from 0 to 8, and the adjacent two substituents can be fused to form a ring.

The term “aromatic ring group” refers to a hydrocarbon group containing an aromatic ring. The term “heteroaromatic ring group” refers an aromatic hydrocarbon group containing at least one heteroatom. The term “fused-ring aromatic group” refers to an aromatic group containing two or more rings, in which two carbon atoms are shared by two adjacent rings, i. e., fused rings. The term “fused heterocyclic aromatic group” refers a fused aromatic hydrocarbon group containing at least one heteroatom. For the purposes of the present disclosure, the aromatic groups or heteroaromatic groups comprise not only aromatic ring systems, but also non-aromatic ring systems. Therefore, systems such as pyridine, thiophene, pyrrole, pyrazole, triazole, imidazole, oxazole, oxadiazole, thiazole, tetrazole, pyrazine, pyridazine, pyrimidine, triazine, carbene, and the like is also considered be aromatic groups or heterocyclic aromatic groups for the purposes of this disclosure.

For the purposes of the present disclosure, the aromatic ring systems contain 5 to 20 ring atoms, the heteroaromatic ring systems contain 1 to 10 carbon atoms and at least one heteroatom, provided that the total number of the carbon atoms and the heteroatoms is at least 4. The heteroatoms are preferably selected from Si, N, P, O, S and/or Ge, particularly preferably selected from Si, N, P, O and/or S.

For the purposes of the present disclosure, the aromatic or heteroaromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be aromatic ring systems for the purposes of this invention.

For the purposes of the present disclosure, the fused-ring aromatic or fused heterocyclic aromatic ring systems contain not only aromatic or heteroaromatic systems, but also have a plurality of aryl or heteroaryl groups linked by short non-aromatic units (<10% of non-H atoms, preferably <5% of non-H atoms, such as C, N or O atoms). Therefore, a system such as 9,9′-spirobifluorene, 9,9-diarylfluorene, triarylamine, diaryl ether, and the like is also considered to be fused-ring aromatic ring systems for the purposes of this invention.

For one of the purposes of the present disclosure, the non-aromatic ring system contains 1 to 10 carbon atoms in the ring system, preferably contains 1 to 3 carbon atoms, including both the saturated ring system and the partially unsaturated ring system, which may be either unsubstituted or substituted with one or more Rₙs, Rₙs may be the same or different at each occurrence and may also comprise one or more heteroatoms, preferably selected from Si, N, P, O, S and/or Ge, particularly preferably from Si, N, P, O and/or S. For example, cyclohexyl-like, piperidine-like systems, or cyclooctadiene-like ring systems. Meanwhile, the term is also applies to fused non-aromatic ring systems.

Specifically, examples of fused-ring aromatic groups include, but not limited to naphthalene, anthracene, fluoranthene, phenanthrene, phenalene, triphenylene, perylene, tetracene, pyrene, benzopyrene, acenaphthylene, fluorene, and derivatives thereof.

Specifically, examples of fused heterocyclic aromatic groups include, but not limited to benzofuran, benzothiophene, indole, carbazole, pyrroloimidazole, pyrrolopyrrole, thienopyrrole, thienothiophene, furopyrrole, furofuran, thienofuran, benzisoxazole, benzoisothiazole, benzimidazole, quinoline, isoquinoline, o-diazanaphthalene, quinoxaline, phenanthridine, primidine, quinazoline, quinazolinone, and derivatives thereof.

The term “linear alkane group” refers to an alkane having carbon atoms linked by a single chain and presenting a linear chain. When there are more than three carbon atoms, the alkane chain can not only be linked by a linear chain, but also can form a branch-like branched structure, this is a branched alkane. When there are more than three carbon atoms, the carbon atoms can form a linear/branched alkane, but also can through a single chain or a double bond linked to form a cyclic alkane, this is an alicyclic hydrocarbon. Alicyclic hydrocarbons can also contain more than two carbon rings, they can be linked in a variety of ways: in the molecule, two rings can share a carbon atom, this system is called the spiro ring; two carbon atoms on the ring can be linked with carbon bridges to form a double or multiple ring system, this is called the bridge ring; a few rings can be linked with each other to form a cage-like structure.

Specifically, examples of C₁-C₈ linear alkane groups include, but not limited to methyl, ethyl, n-propyl, n-butyl, n-pentyl, n-hexyl, n-heptyl, n-octyl.

Specifically, examples of C₁-C₈ branched alkane groups include, but not limited to isopropyl, t-butyl, isopentane, neopentane, 1,2-dimethylcyclohexane, trimethylpropane, 2,3-dimethylbutane, 2,2-dimethylbutane, 2-methylhexane, 3-methylhexane, 2,2-dimethylpentane, 3,3-dimethylpentane, 2,3-dimethylpentane, 2,4-dimethylpentane, 3-ethylpentane, 2,2,3-trimethylbutane, 2-methylheptane, 3-methylheptane, 4-methylheptane, 3-ethylhexane, 2,2-dimethylhexane, 2,3-dimethylhexane, 2,4-dimethylhexane, 2,5-dimethylhexane, 3,3-dimethylhexane, 3,4-dimethylhexane, 2-methyl-3-ethylpentane, 3-methyl-3-ethylpentane, 2,2,3-trimethylpentane, 2,2,4-trimethylpentane, 2,3,3-trimethylpentane, 2,3,4-trimethylpentane, 2,2,3,3-tetramethylbutane, and derivatives thereof.

Specifically, examples of C₃-C₈ alicyclic hydrocarbon groups include, but not limited to cyclopropane, cyclobutane, methylcyclopropane, cyclopentane, cyclohexane, cycloheptane, 1,2-dimethylcyclopentane, 1-methyl-3-ethylcyclopentane, cyclooctane, cyclopentene, cyclooctyne, 1,3-cyclohexadiene, 1-methyl-1-cyclohexene, 3-methyl-1-cyclohexene, 3-methylcyclopentene, 1,6-dimethyl-1-cyclohexene, 5-methyl-1,3-cyclohexene, spiro[2.4]heptane, 5-methylspiro[2.4]heptane, bicyclo[2.2.1]heptane, bicyclo[2.1.0]pentane, bicyclo[3.1.1]heptane, and derivatives thereof.

The term “alkoxy group” refers to the combination of an alkyl group with an oxygen atom, which can be further classified according to the type of alkyl group into a linear/branched alkyl group linked to an oxygen such as methoxy, ethoxy, propoxy, tert-butoxy, etc. and a cycloalkane linked to an oxygen such as cyclopropoxy, cyclohexyloxy, etc.

Specifically, examples of C₁-C₈ alkoxy groups include, but not limited to methoxy, ethoxy, propoxy, 2-methylethoxy, cyclopropoxy, n-butoxy, tert-butoxy, cyclobutoxy, 2-methylpropoxy, 3-methylpropoxy, n-pentyloxy, cyclopentyloxy, isopentyloxy, neopentyloxy, dimethylhexyloxy, trimethylpropoxy, n-hexyloxy, cyclohexyloxy, 2,3-dimethylbutoxy, 2,2-dimethylbutoxy, 2-methylhexyloxy, 3-methylhexyloxy, 2,2-dimethylpentyloxy, 3,3-dimethylpentyloxy, 2,3-dimethylpentyloxy, 2,4-dimethylpentyloxy, 3-ethylpentyloxy, n-heptyloxy, cycloheptyloxy, 2-methylheptyloxy, 3-methylheptyloxy, 4-methylheptyloxy, n-octyloxy, cyclooctyloxy, 3-ethylhexyloxy, 2,2-dimethylhexyloxy, 2,3-dimethylhexyloxy, 2,4-dimethylhexyloxy, 2,5-dimethylhexyloxy, 3,3-dimethylhexyloxy, 3,4-dimethylhexyloxy, 2-methyl-3-ethylpentyloxy, 3-methyl-3-ethylpentyloxy, and derivatives thereof.

In some embodiments, B and C of the organic compound as represented by formula (I) are the same or different at each occurrence and are independently selected from a substituted/unsubstituted benzene ring, biphenyl, naphthalene, anthracene, phenanthrene, fluoranthene, pyrene, fluorene, pyridine, dibenzofuran, etc., where the substituents of the aforementioned substituted structures are identically defined as the above-mentioned R₁.

In some embodiments, the organic compound comprises a structure of any one of formulas (II-a)-(II-f):

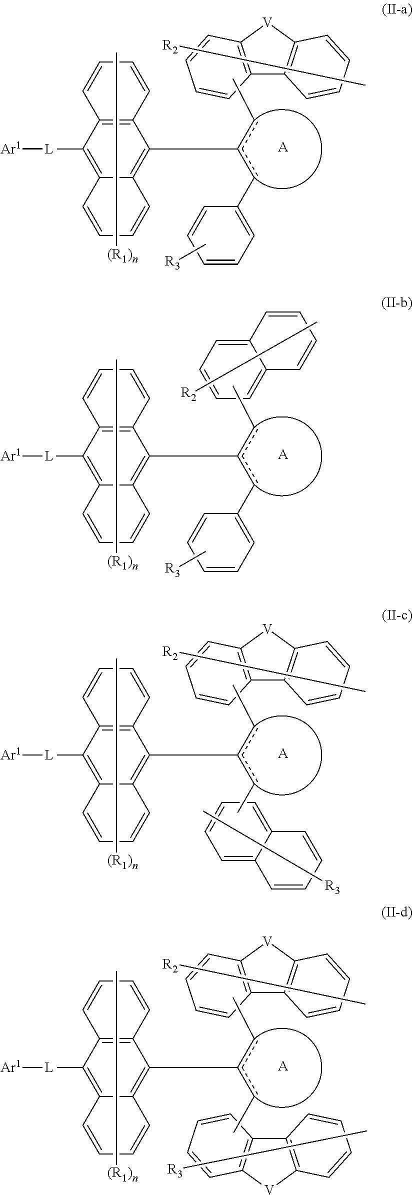

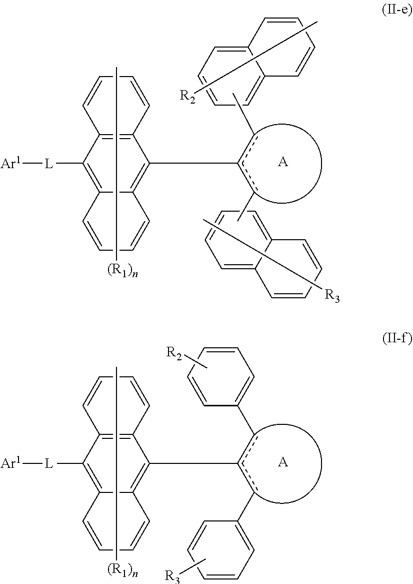

Where each A is independently selected from the group consisting of a substituted/unsubstituted fused aromatic or heteroaromatic ring structure containing 5 to 30 ring atoms, the two groups are each linked to A at the adjacent position of the linkage site between A and anthracene unit, and each A may be linked or fused with any adjacent linking group to form a ring; V, in multiple occurrences, is independently selected from B(R₁₀), C(═O), N(R₁₁), C(R₁₀R₁₁), O, S, P, P═O, or P═S; Ar¹, L, n, and R₁ are identically defined as described above; R₂, R₃, R₁₀, and R₁₁ are identically defined as R₁.

Preferably, V is selected from B(R₁₀), C(═O), N(R₁₁), O, or S.

In some embodiments, the As of the organic compound as represented by formulas (II-a)-(II-f) are independently selected from a substituted/unsubstituted benzene ring, biphenyl, naphthalene, anthracene, phenanthrene, fluoranthene, pyrene, fluorene, pyrrole, furan, thiophene, pyridine, cyclopentadiene, dibenzofuran, etc., where the substituents of the aforementioned substituted structures are identically defined as the above-mentioned R₁.

In some embodiments, the organic compound is shown in one of the following formulas:

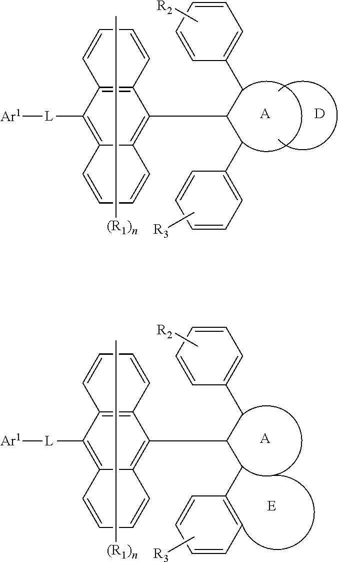

Where A, Ar¹, L, n and R₁-R₃ are identically defined as described above; D denotes a C₆-C₃₀ aromatic group or a C₅-C₃₀ heteroaromatic group being linked or fused with the A to form a ring; each E denotes an A being fused with the benzene ring linked to the A and its derivatives to form a ring.

In some embodiments, the organic compound is shown in formulas (III-a)-(III-c):

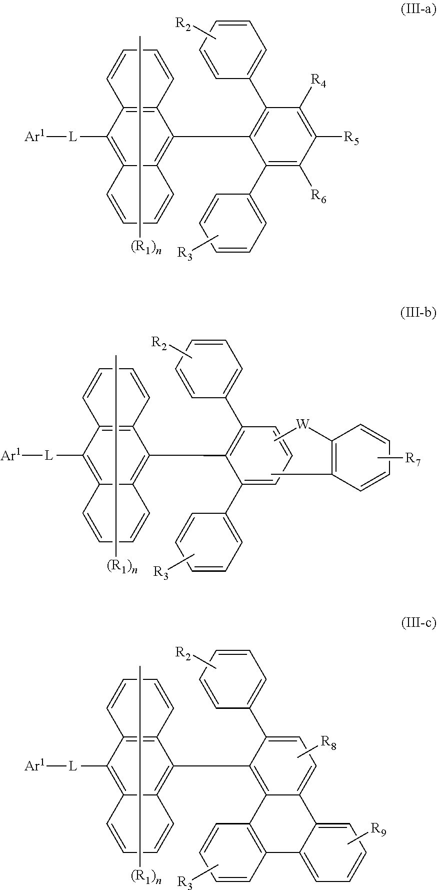

Where W, in multiple occurrences, is independently selected from B(R₃₀), C(═O), N(R₃₁), O, S, P, P═O, or P═S; R₂-R₉, R₃₀, R₃₁ are identically as the above-mentioned R₁, while R₄ and R₆ may be independently fused with R₅ to form a ring; Ar¹, L, n, and R₁ are identically defined as described above.

In some embodiments, L of the organic compound may be same or different, and is selected from a single bond, one or combinations of more than one of the following structural groups:

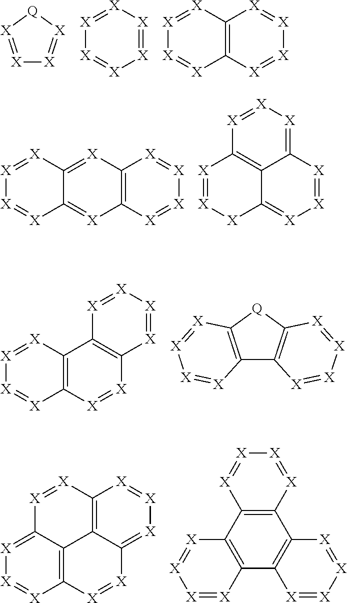

Where X, in multiple occurrences, is independently selected from C(R₂₇) or N; Q, in multiple occurrences, is independently selected from B(R₂₈), C(═O), N(R₂₉), O, S, P, P═O, or P═S; R₂₇-R₂₉ are identically defined as the above-mentioned R₁.

In some embodiments, Ar¹ of the polycyclic aromatic compound may be same or different, and is selected from one or combinations of more than one of the following structural groups:

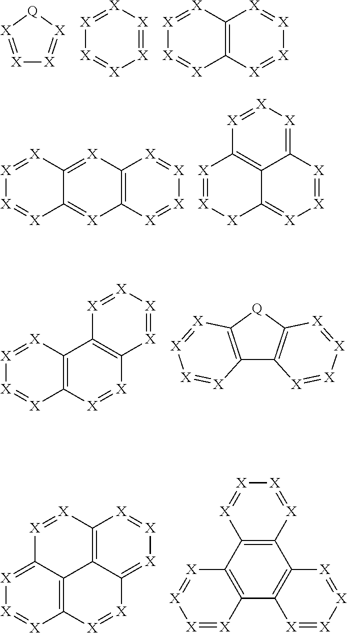

Where X, Q are identically defined as described above.

In some embodiments, each Ar¹ is independently selected from a substituted/unsubstituted benzene, naphthalene, anthracene, phenanthrene, fluoranthene, pyrene, fluorene, pyrrole, furan, thiophene, pyridine, cyclopentadiene, or dibenzofuran.

In some embodiments, each Ar¹ is independently selected from one of the structures of the following formulas:

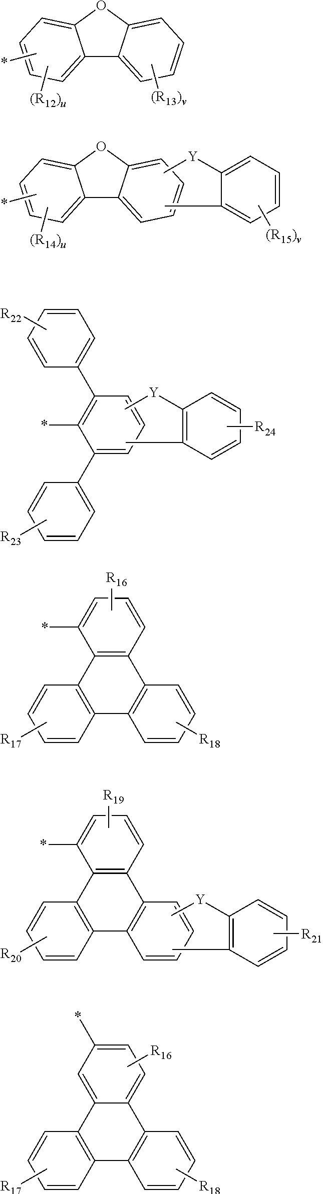

Where Y, in multiple occurrences, is independently selected from B(R₂₅), C(═O), N(R₂₆), C(R₂₅R₂₆), O, S, P, P═O, or P═S; * denotes a linkage site; R₁₂-R₂₆ are identically defined as the above-mentioned R₁, u, v are identically defined as the above-mentioned n, and when a plurality of substituents (R₁₂-R₂₆) are present at the same time, the adjacent two substituents can be fused to form a ring.

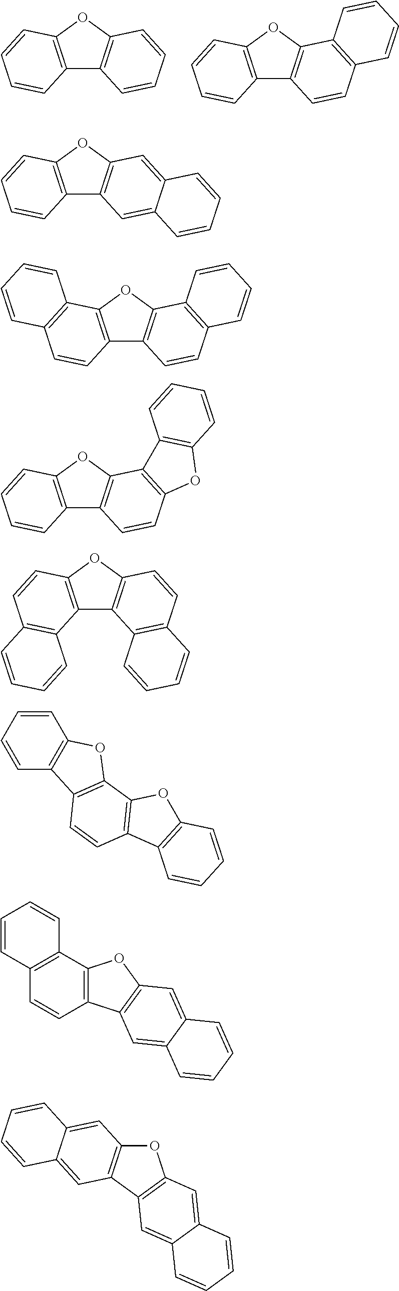

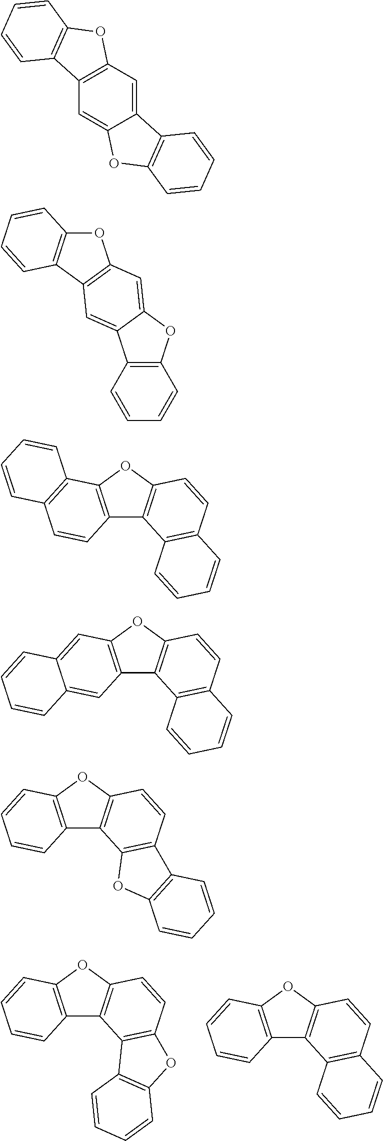

In some embodiments, the organic compound comprises one of the structures of the following formulas:

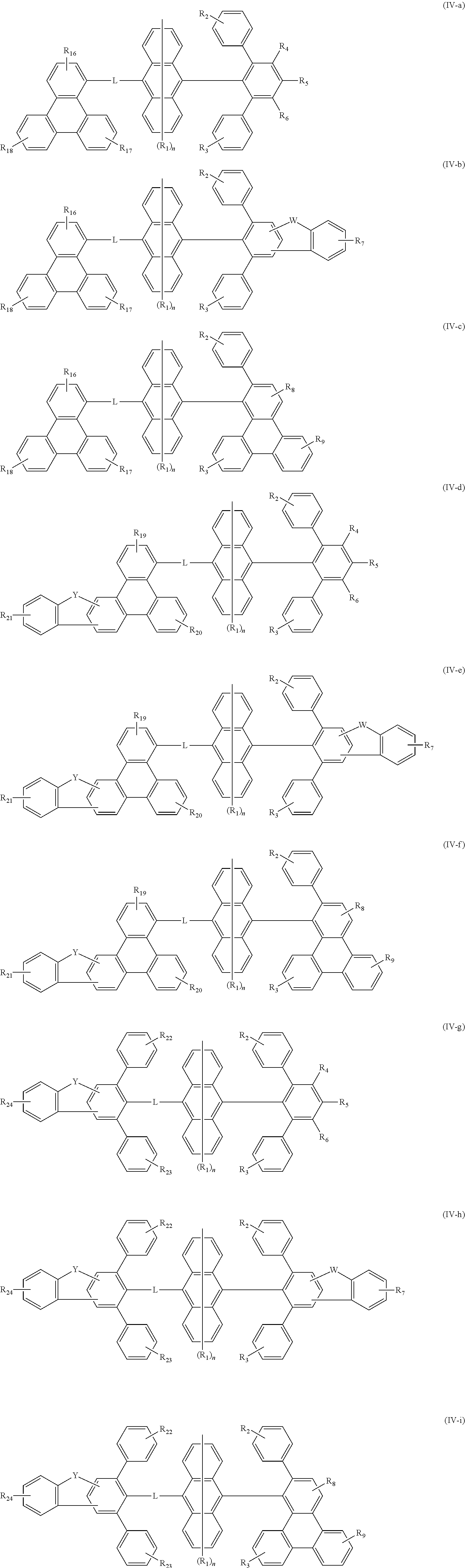

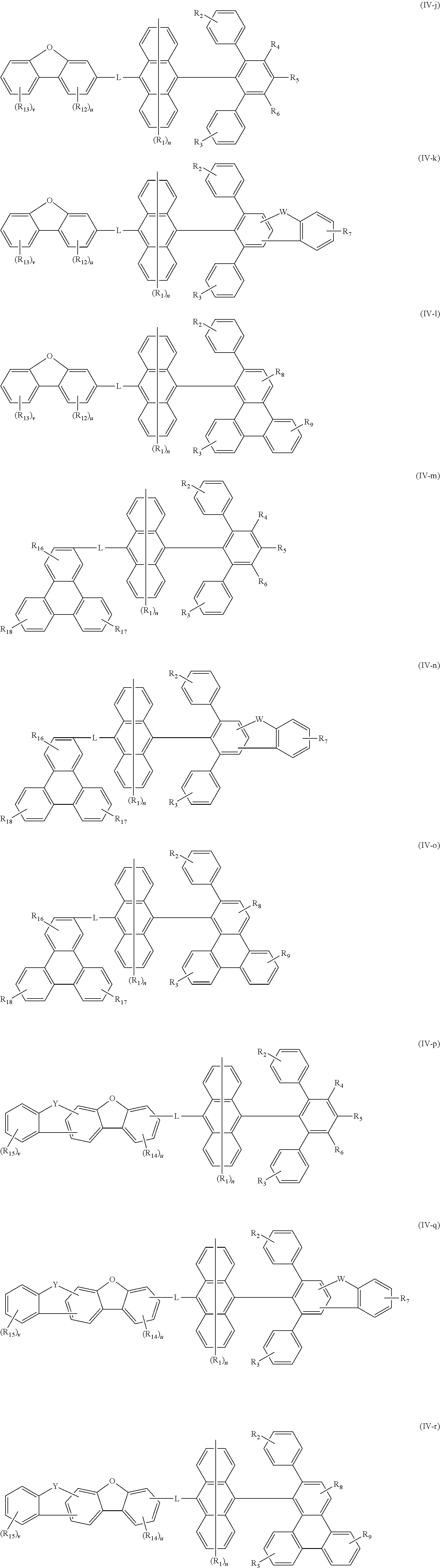

Where W, Y, L, R₁-R₉, R₁₂-R₂₄, n, u, and v are identically defined as described above.

In some embodiments, each L or Ar¹ may be independently selected from one or combinations of more than one of the following structural groups, where H atoms on the ring may be substituted arbitrarily:

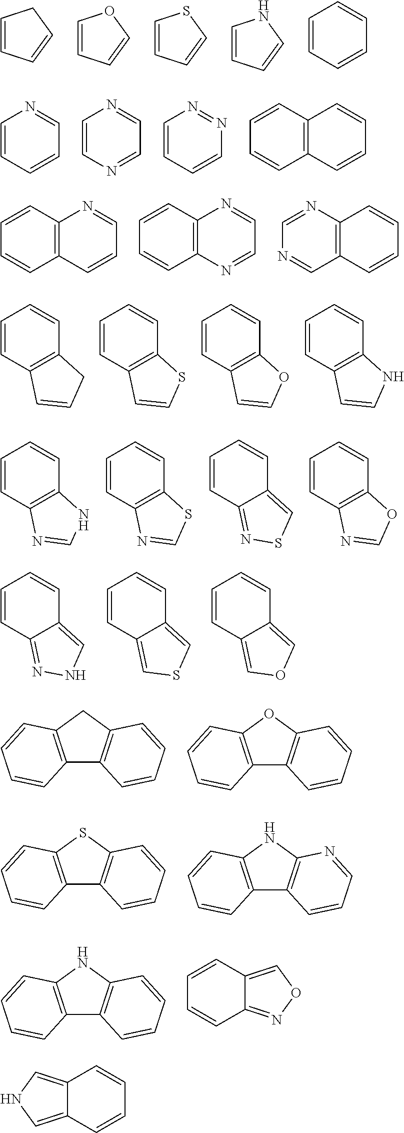

In some embodiments, R₁-R₃₁ may be further selected from one or combinations of more than one of the following structural groups, where H atoms on the ring may be substituted arbitrarily:

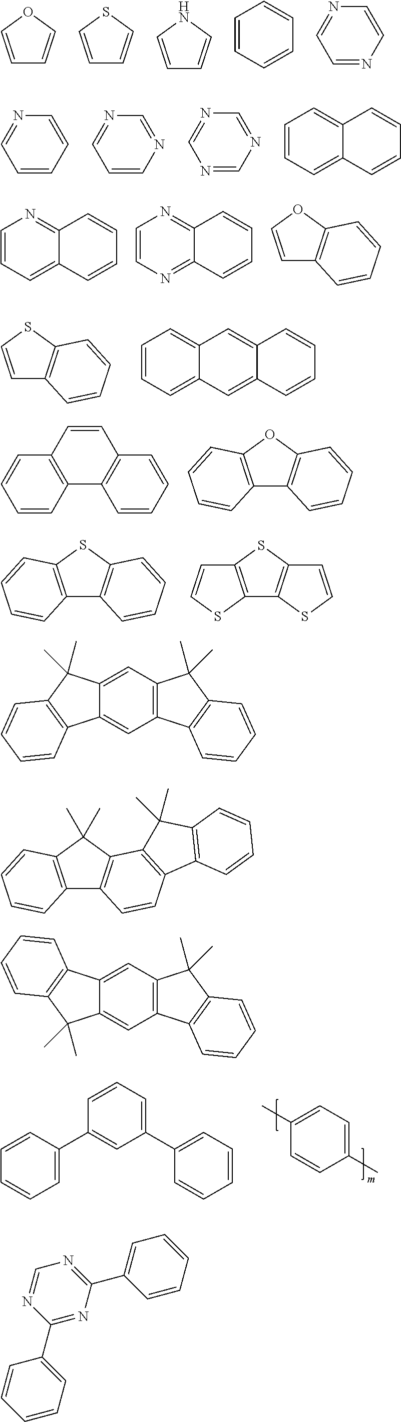

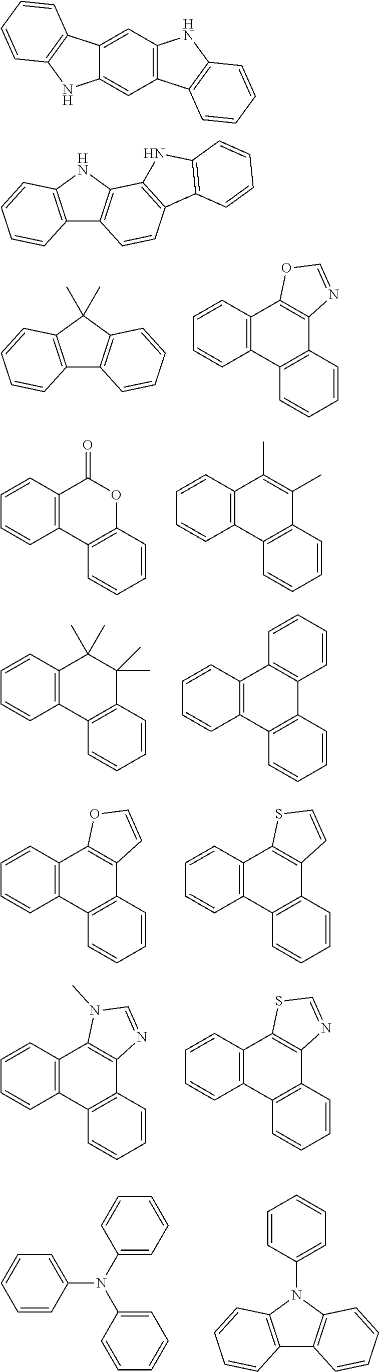

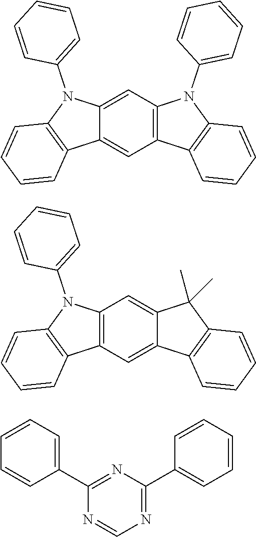

Where m is 1, or 2, or 3, or 4.

In some embodiments, R₁-R₃₁ may be further selected from one or combinations of more than one of the following structural groups, where H atoms on the ring may be substituted arbitrarily:

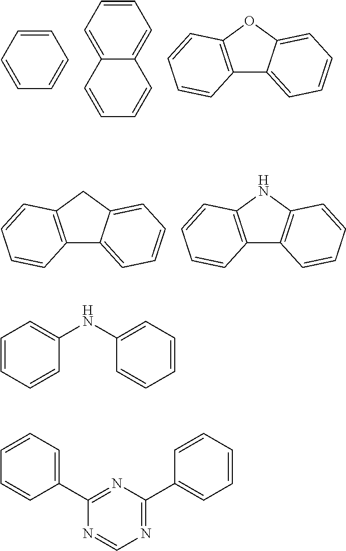

In some embodiments, R₁-R₃₁ of the organic compound may be bridged to a carbon atom on an adjacent or linked aromatic ring, the bridging groups being the same or different as a single bond, or a di-bridged, or tri-bridged linkage.

In some embodiments, R₁-R₃₁ of the organic compound at each occurrence are independently selected from a substituted/unsubstituted C₆-C₂₀ aromatic ring, a C₅-C₂₀ heteroaromatic ring, a C₁₀-C₂₀ fused ring, a C₁-C₈ linear/branched alkane, a C₃-C₁₀ alicyclic hydrocarbon, a C₁-C₈ alkoxy group or an allyl group.

Depending on the substitution type, the organic compound of formula (I) may have various functions, including, but not limited to, hole-transport function, electron-transport function, light-emitting function, and exciton-blocking function. For example, in formulas (III-a)-(III-c), the choice of R₁-R₃₁ determines the suitable function of the organic compounds, as R₁-R₃₁ have an influence on the electronic characteristics of the compounds in formulas (III-a)-(III-c).

In some embodiments, the organic compound as described herein is partially deuterated; preferably 10% or more of total H, more preferably 20% or more of total H, further preferably 30% or more of total H, and most preferably 40% or more of total H, are deuterated.

Specific structures of organic compounds as described herein are listed below (but not limited to), which can be substituted at all possible substitution sites.

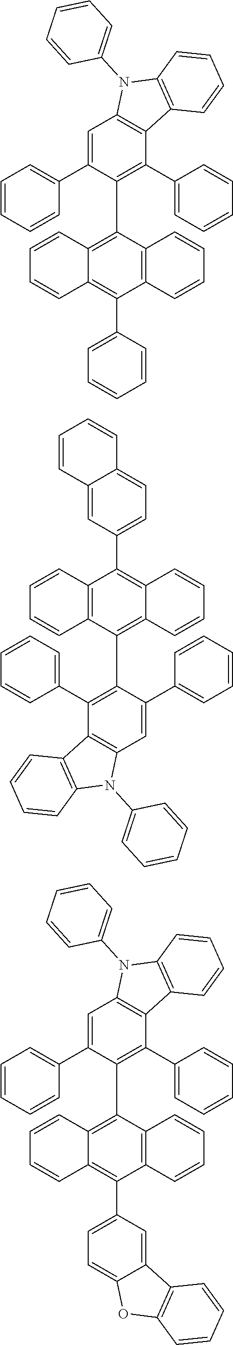

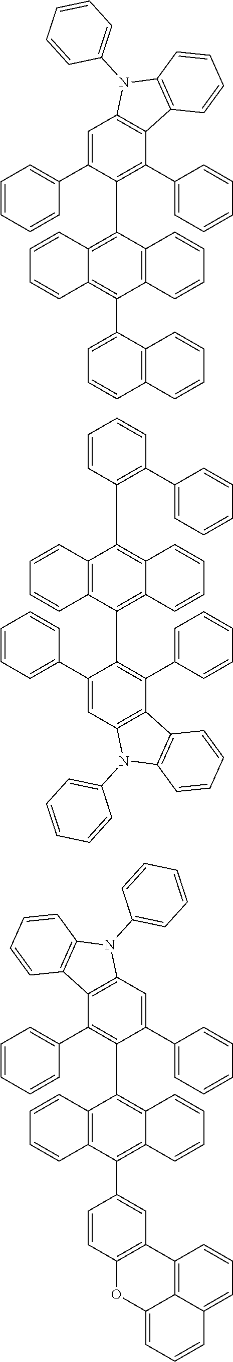

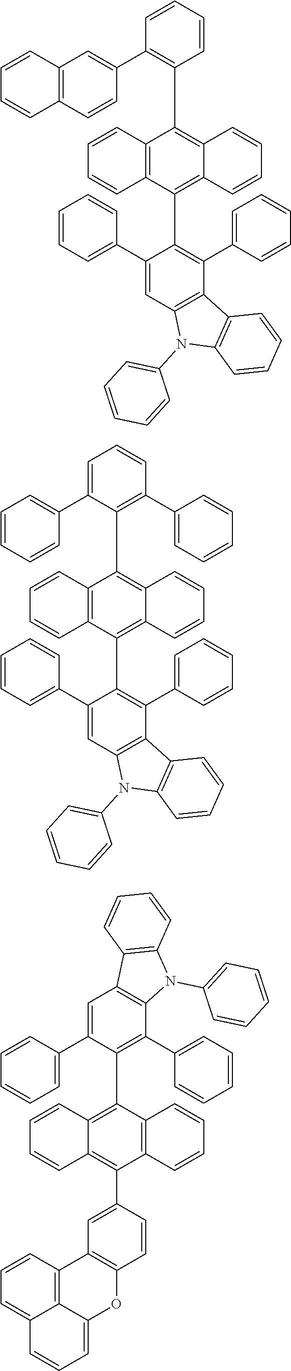

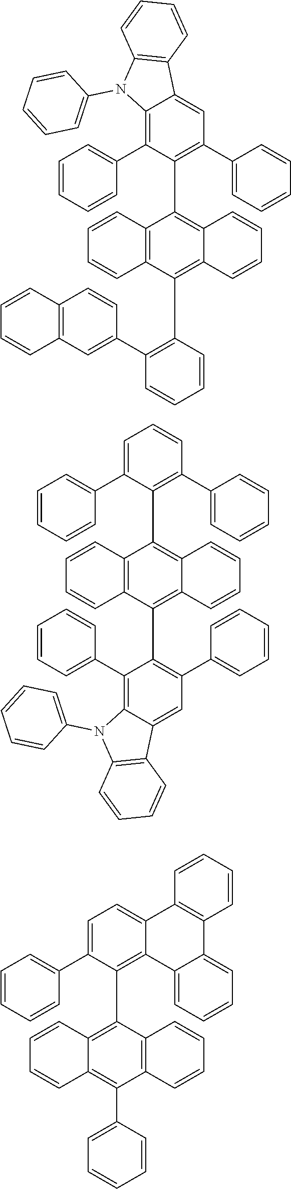

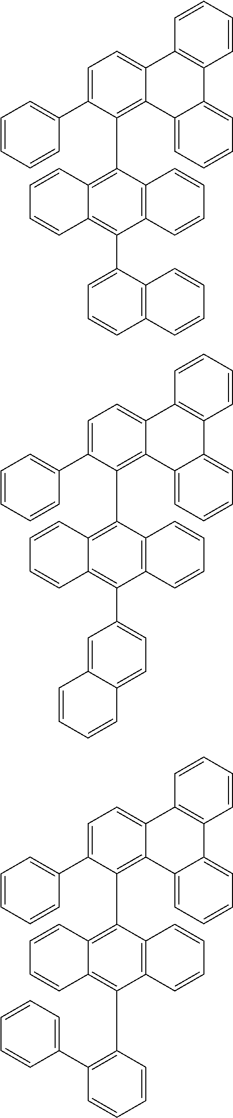

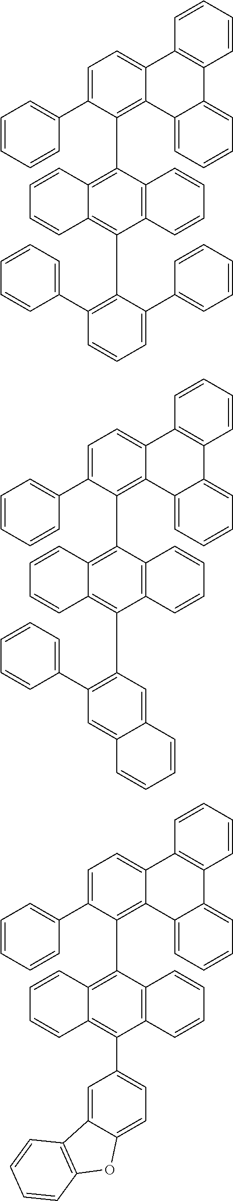

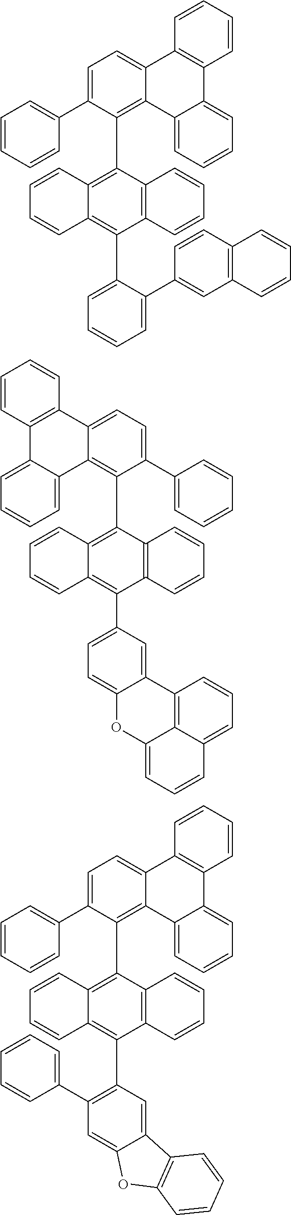

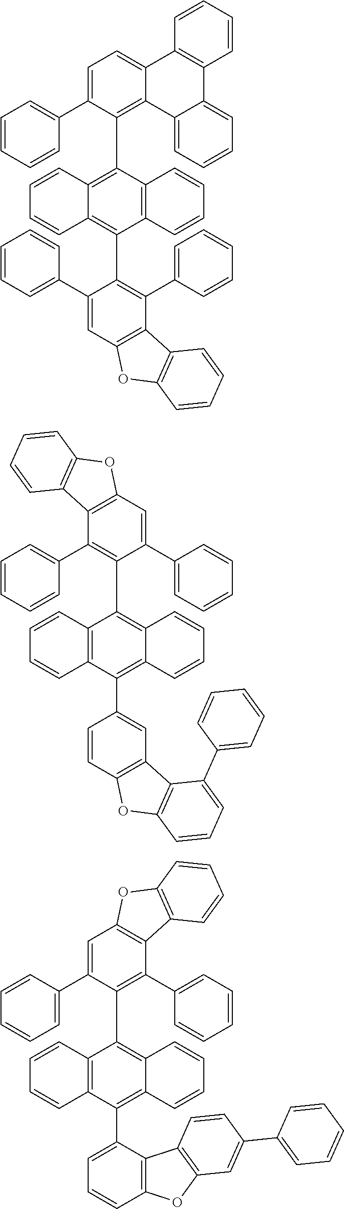

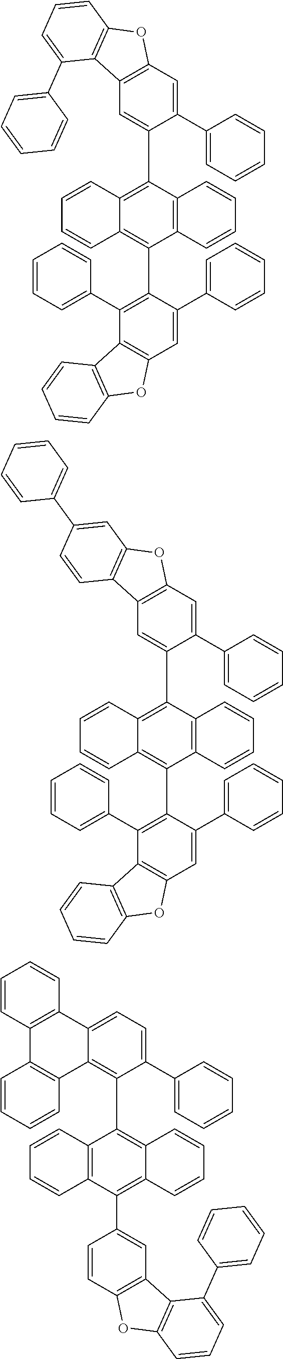

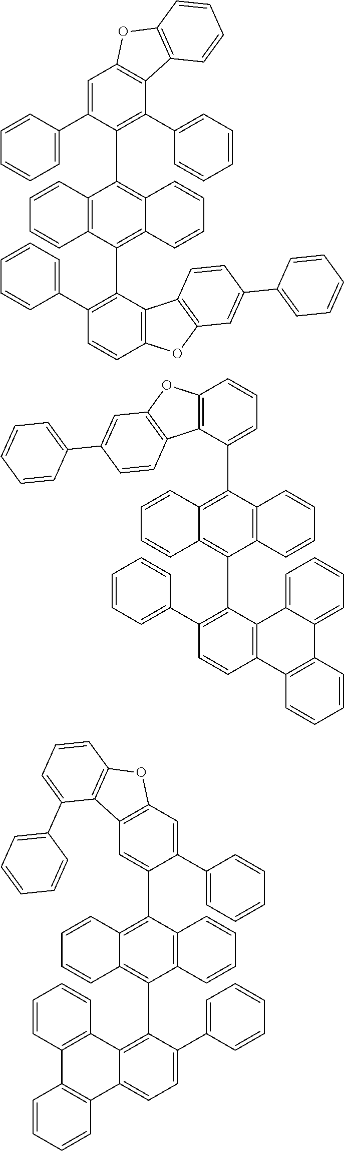

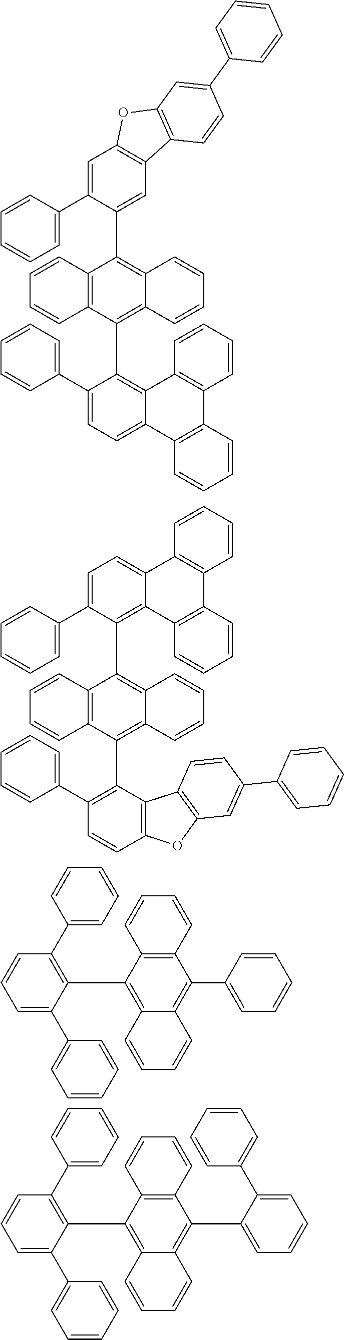

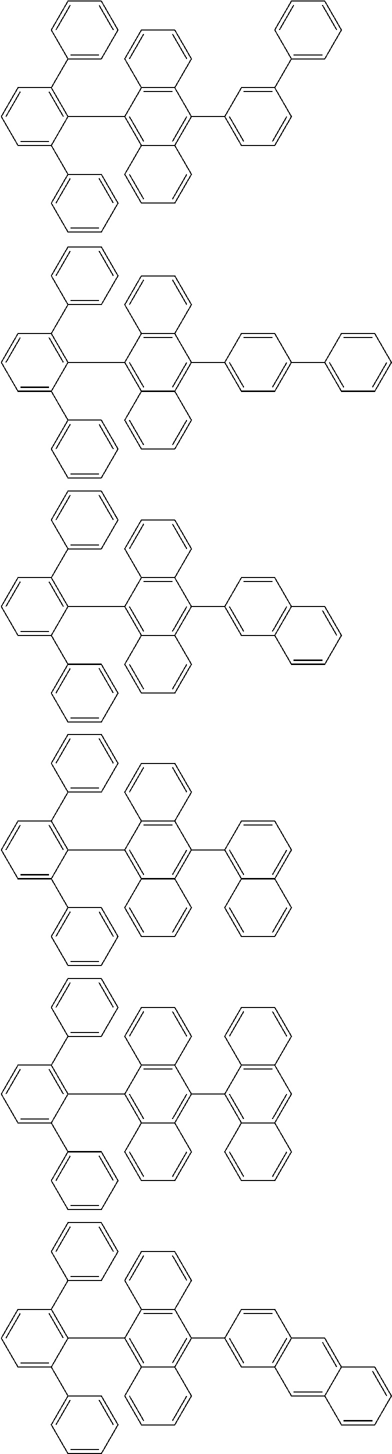

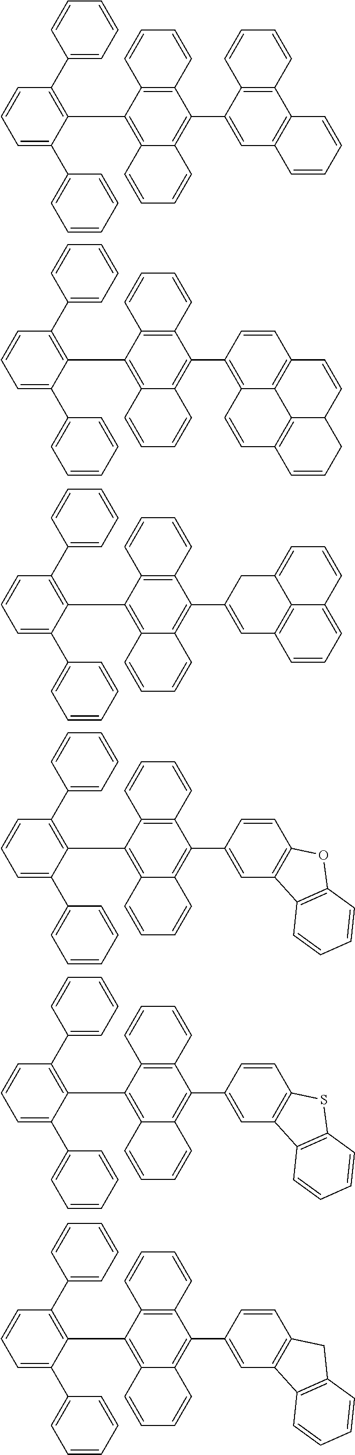

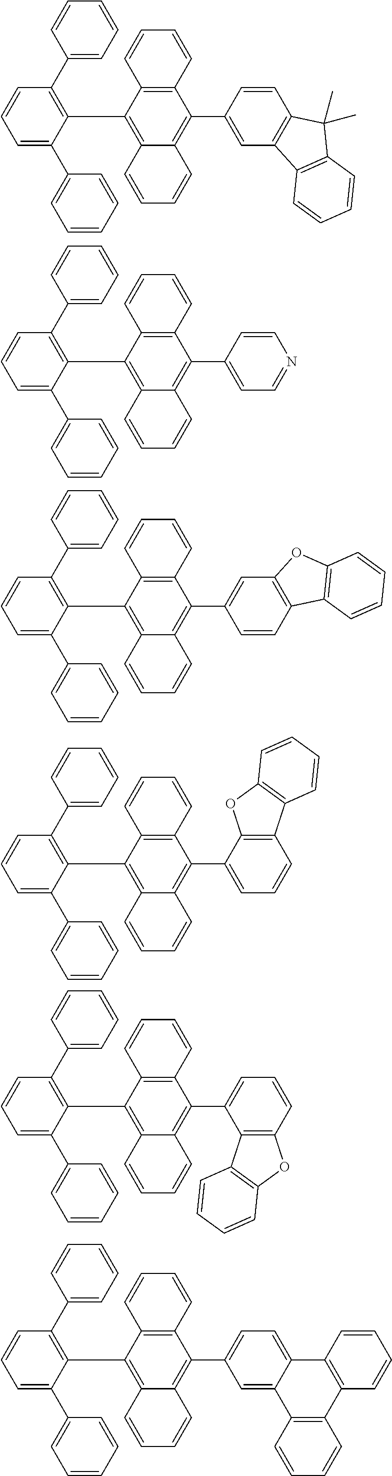

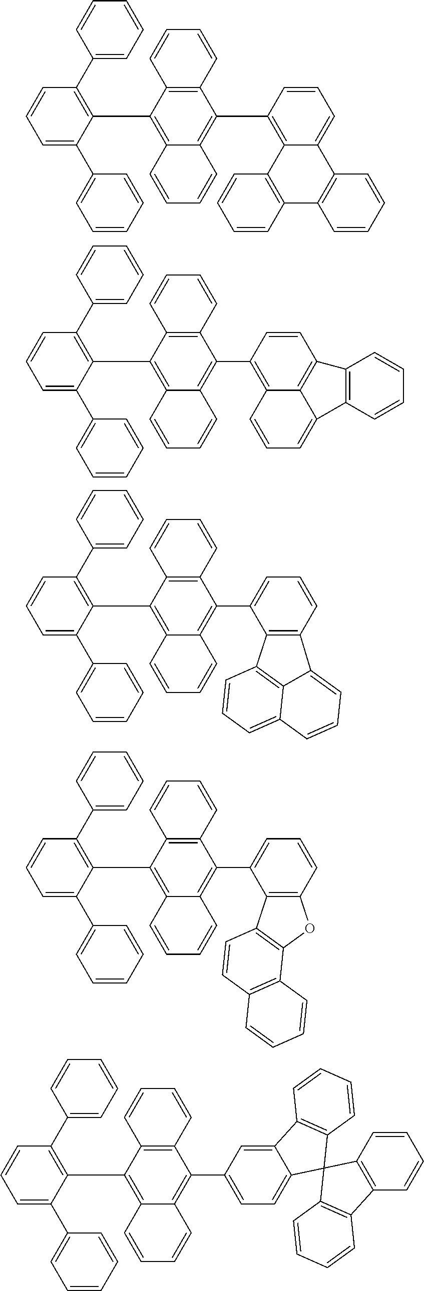

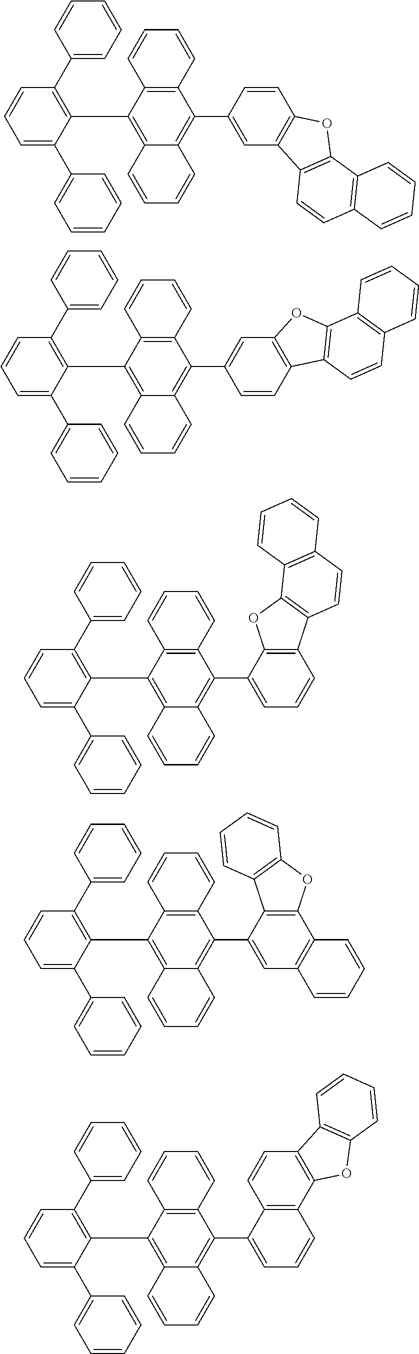

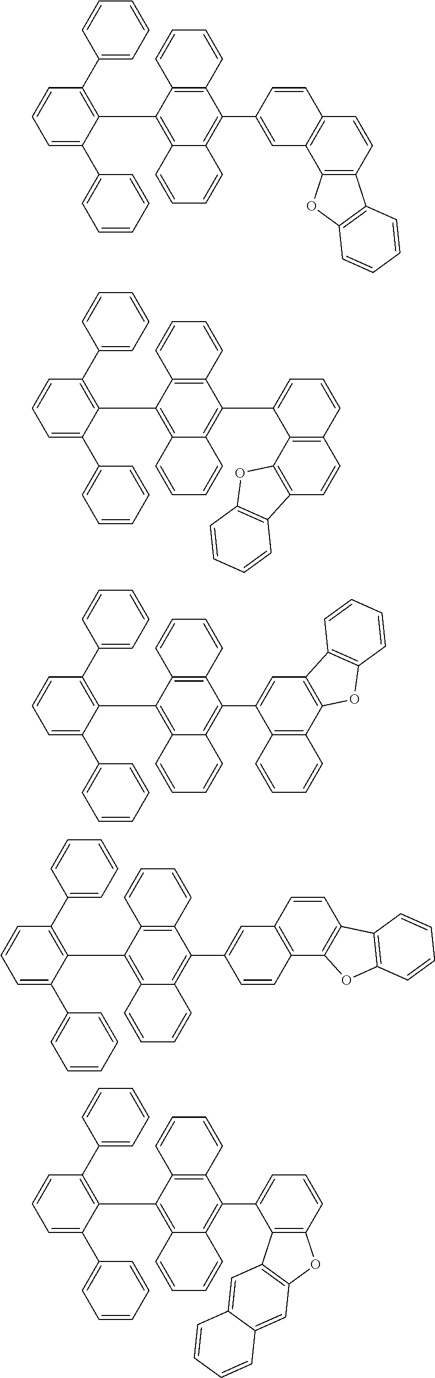

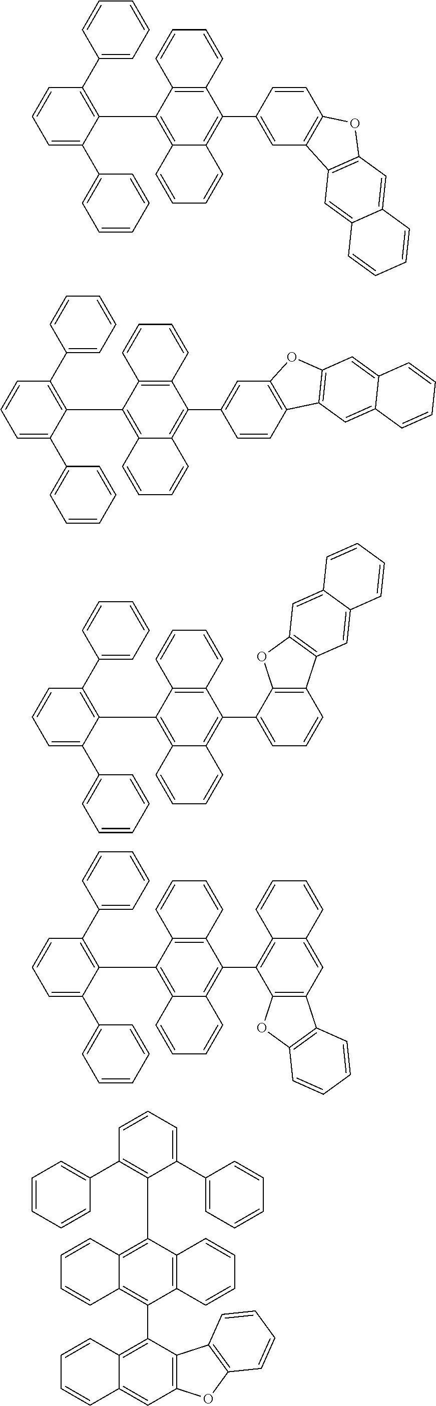

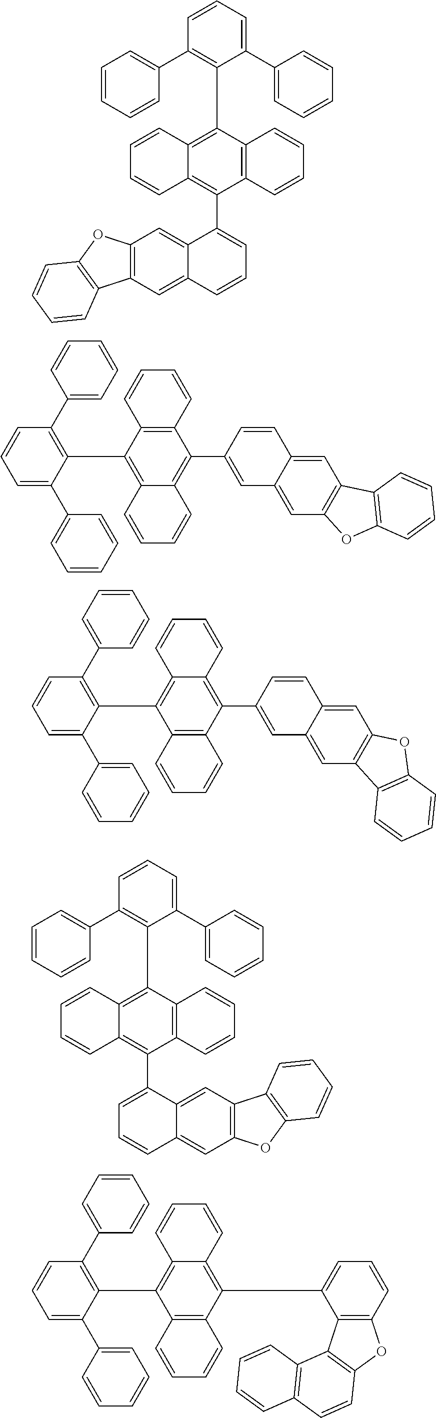

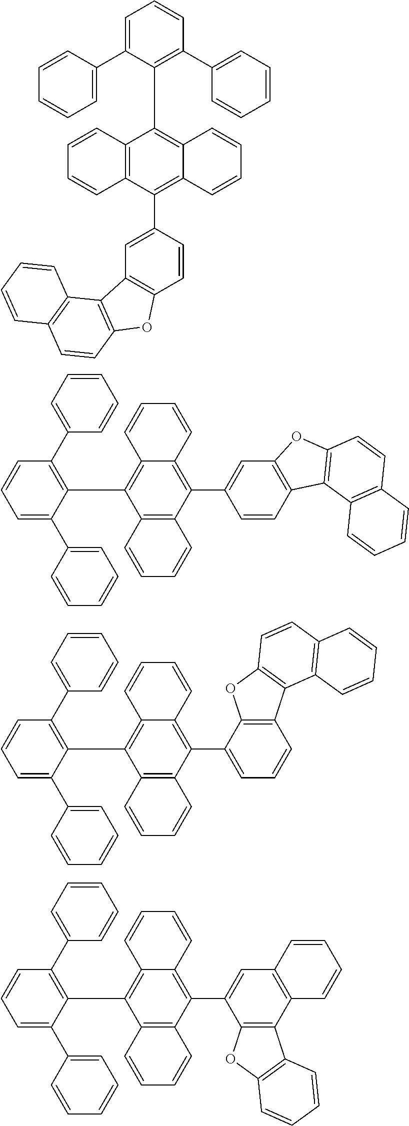

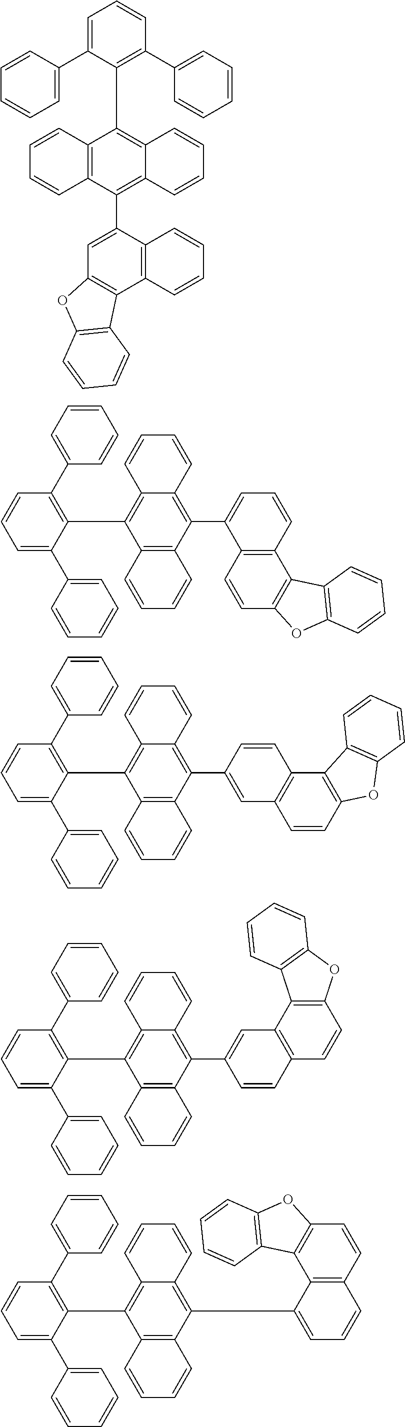

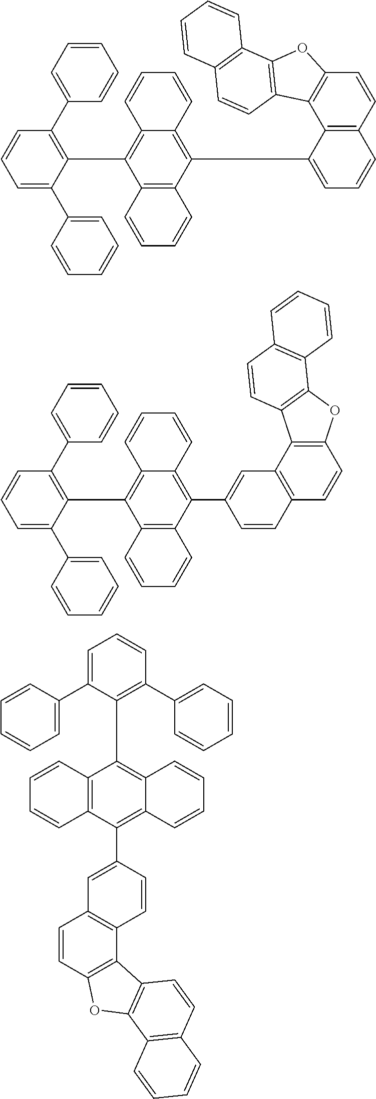

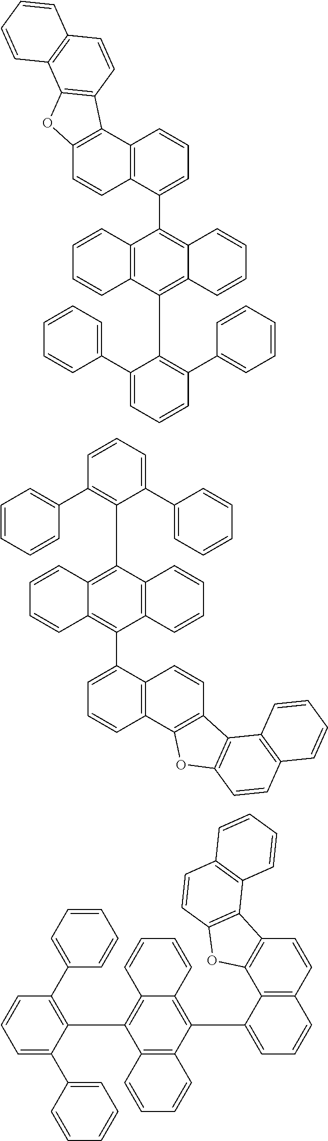

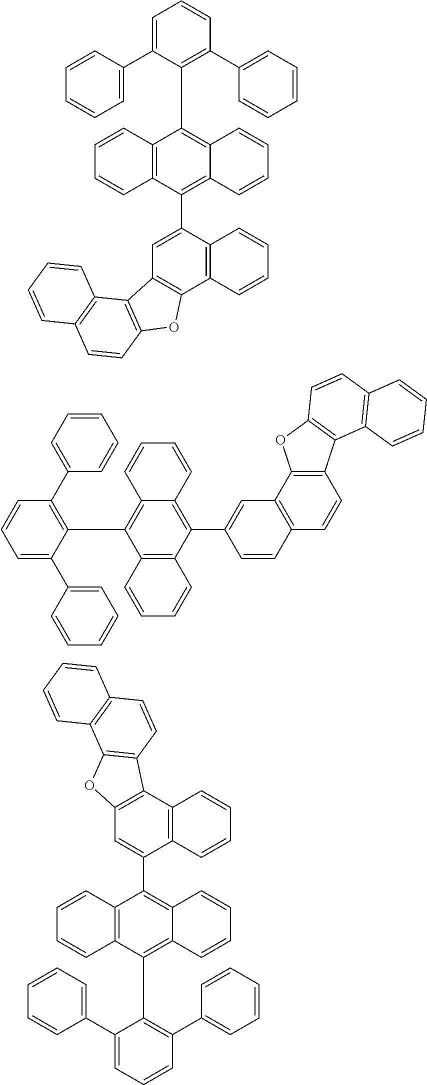

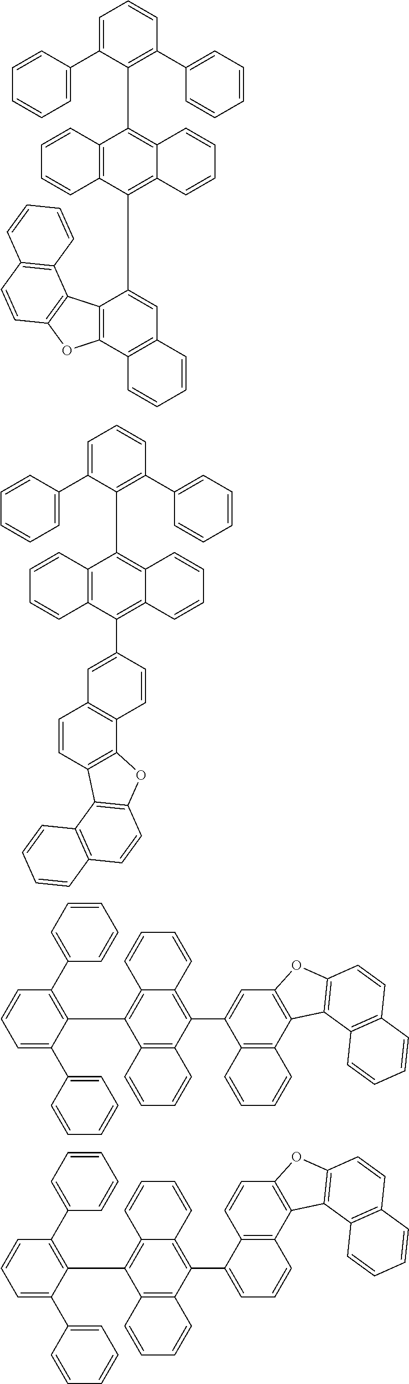

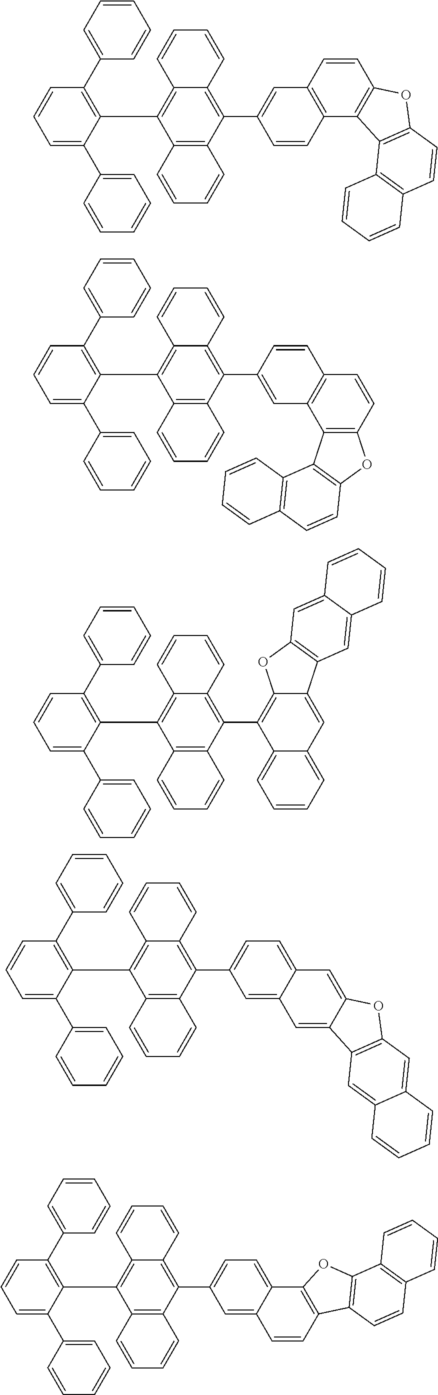

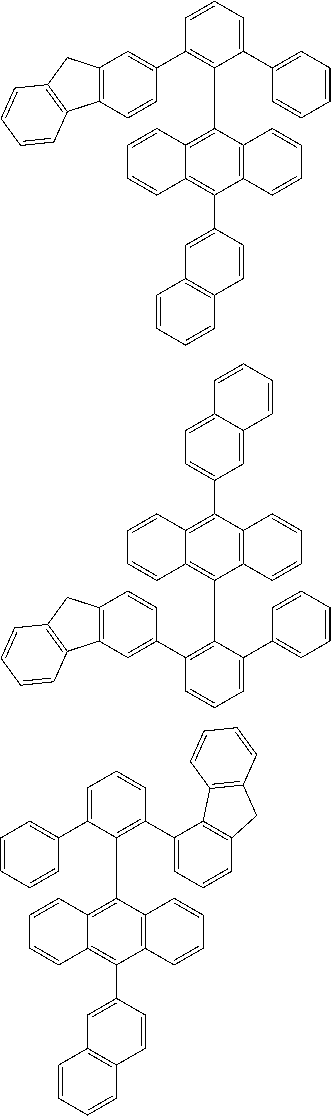

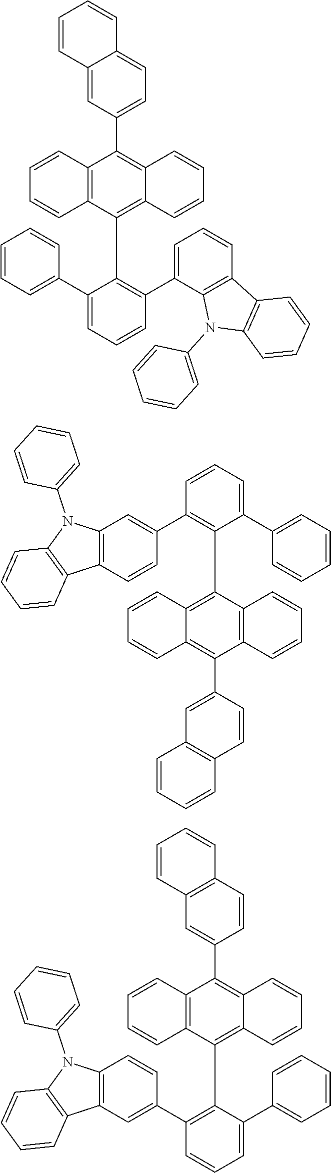

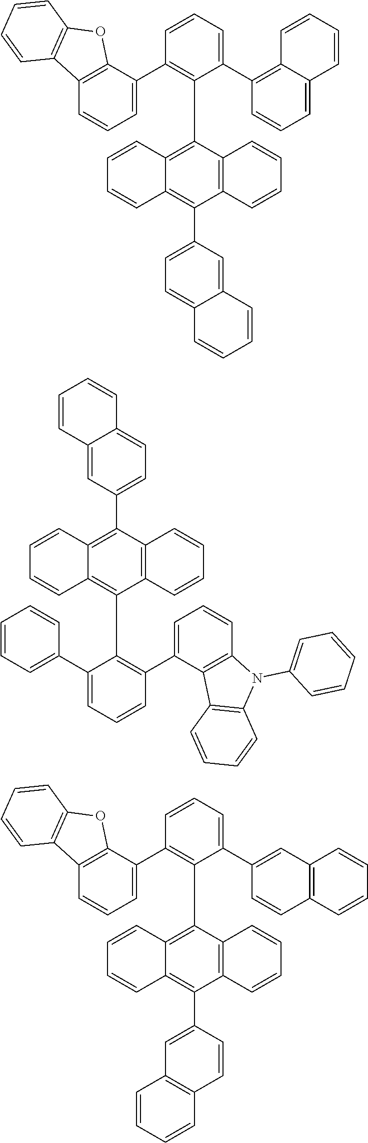

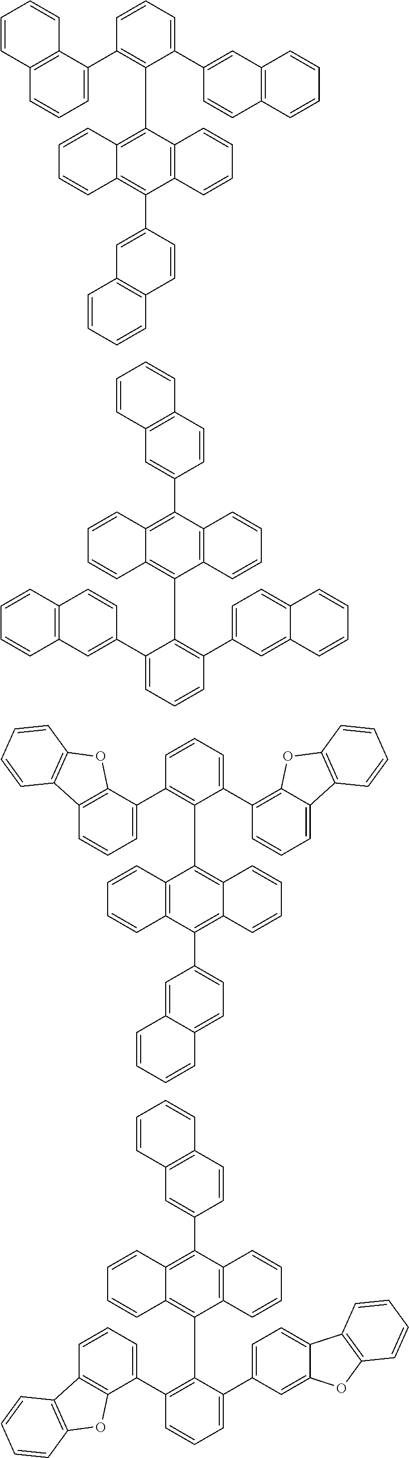

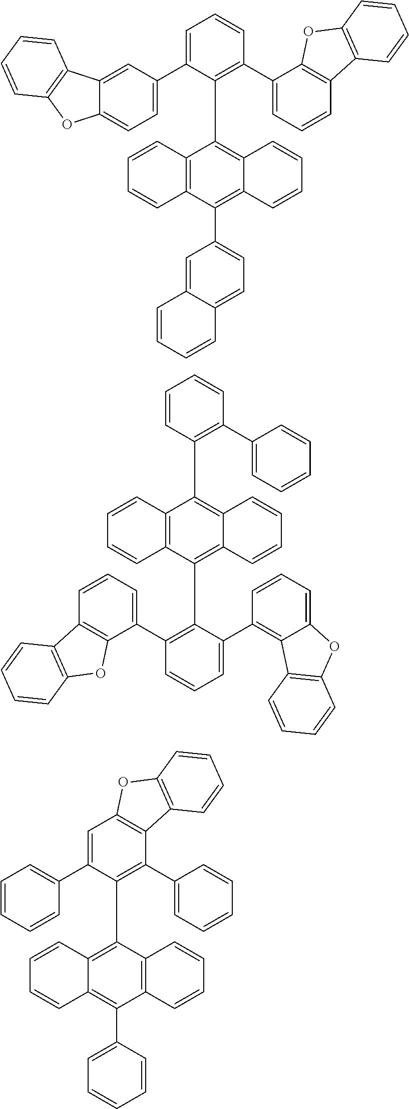

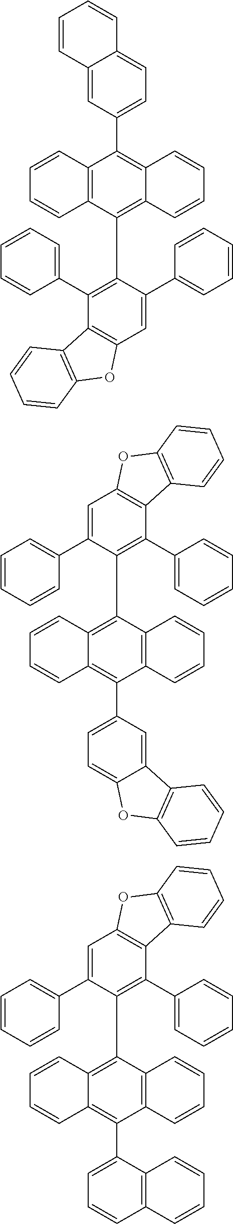

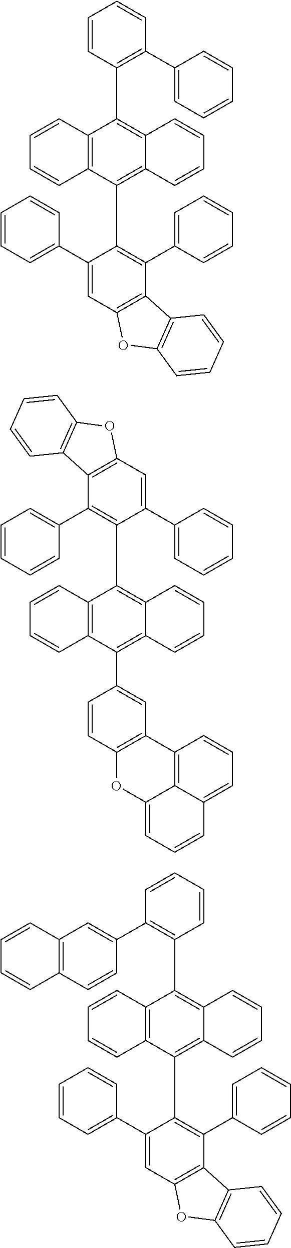

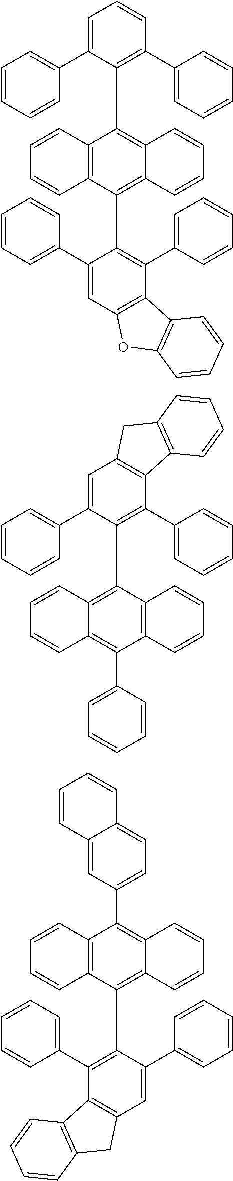

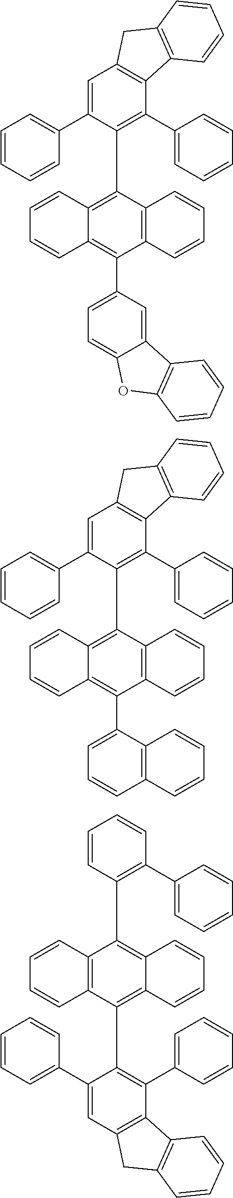

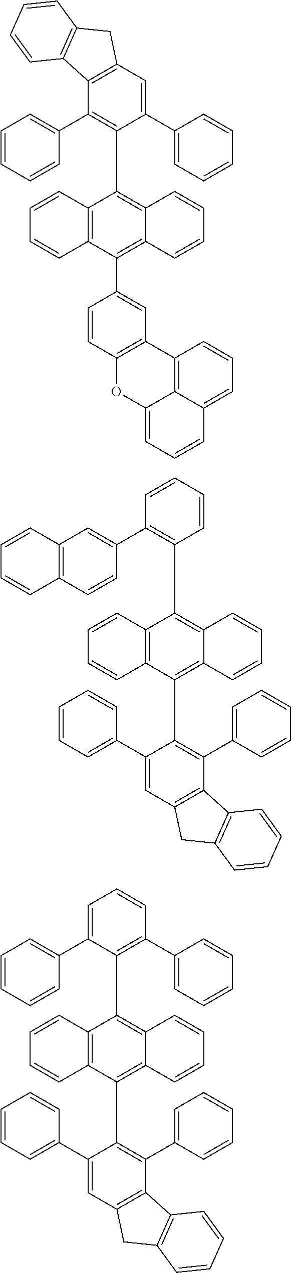

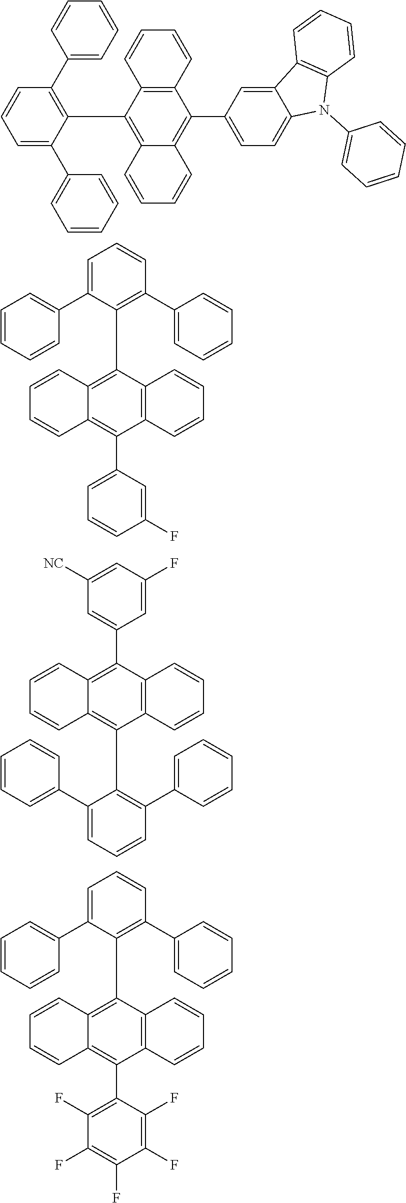

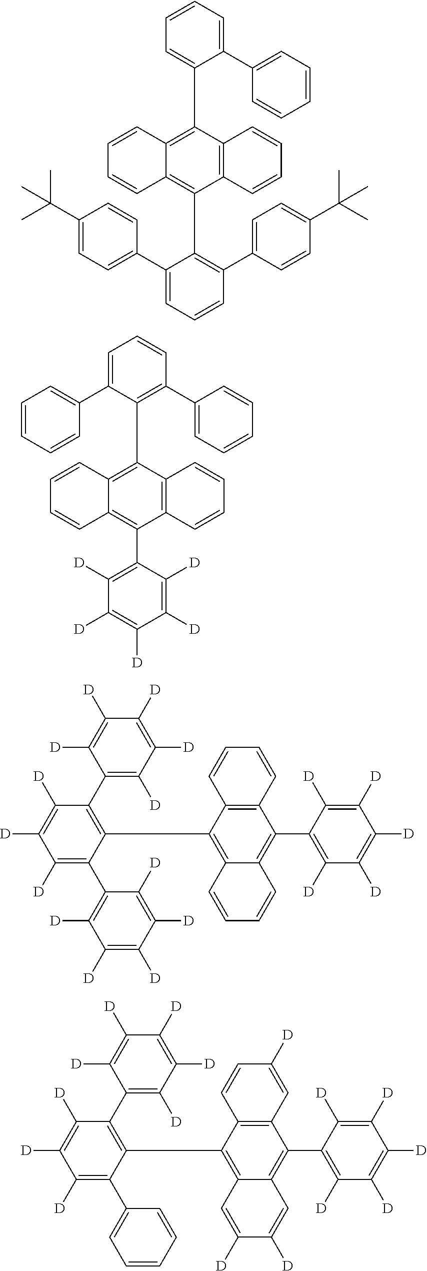

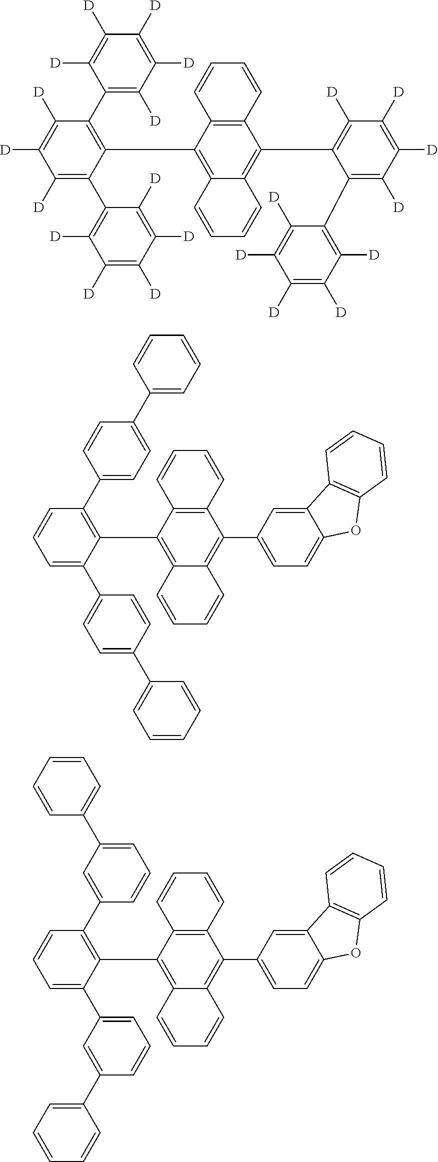

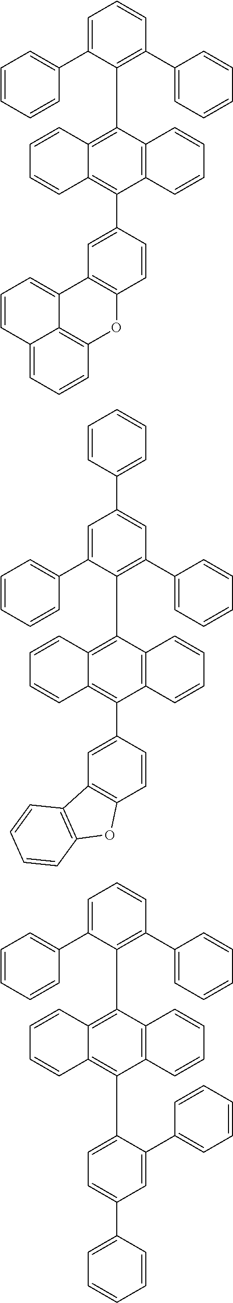

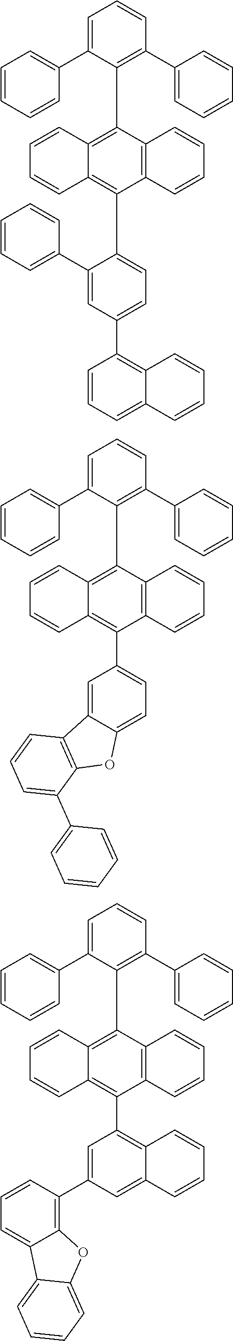

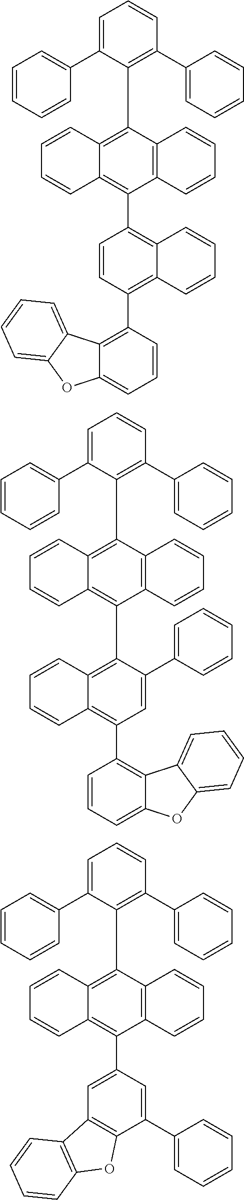

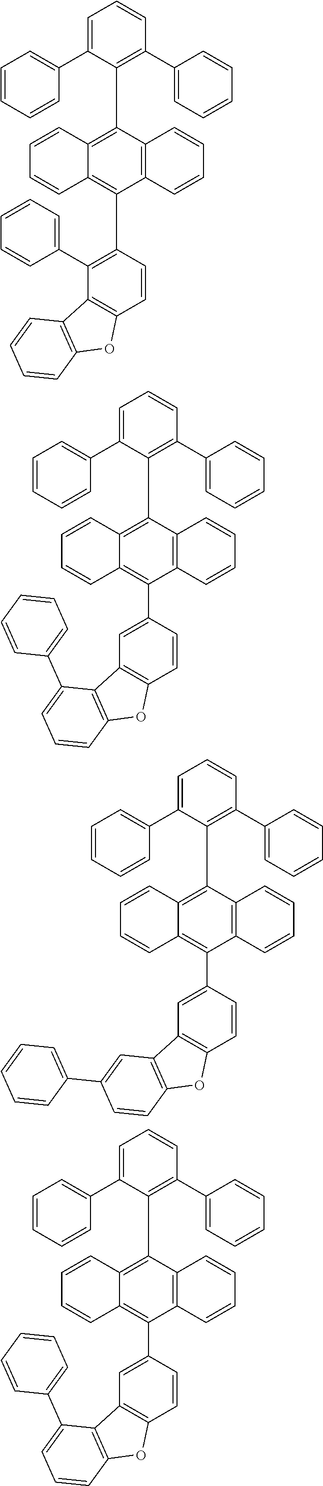

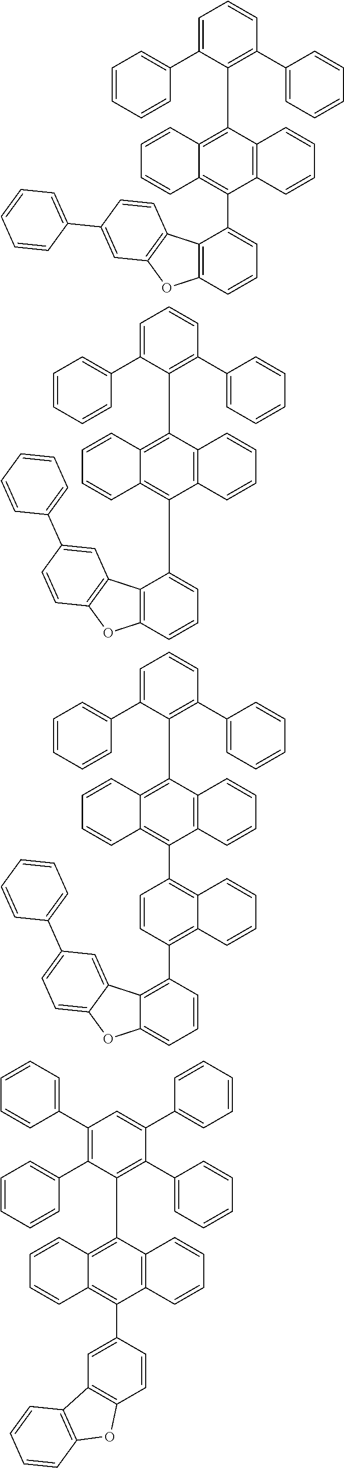

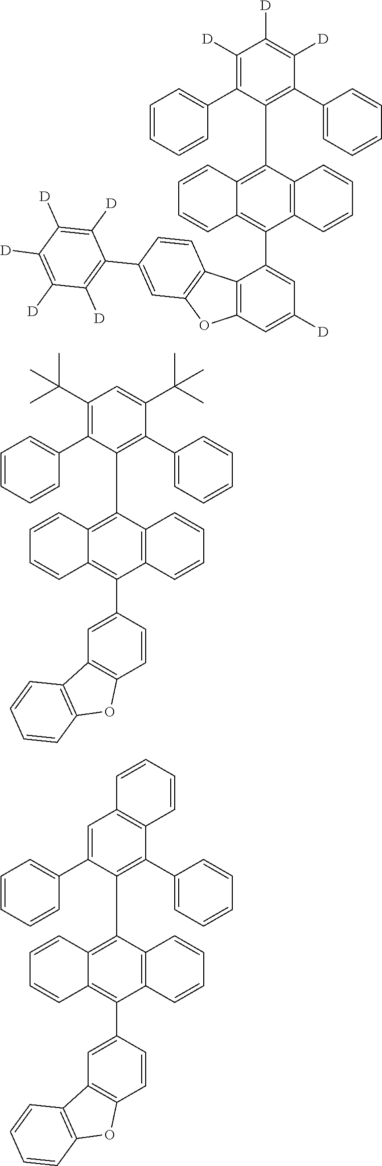

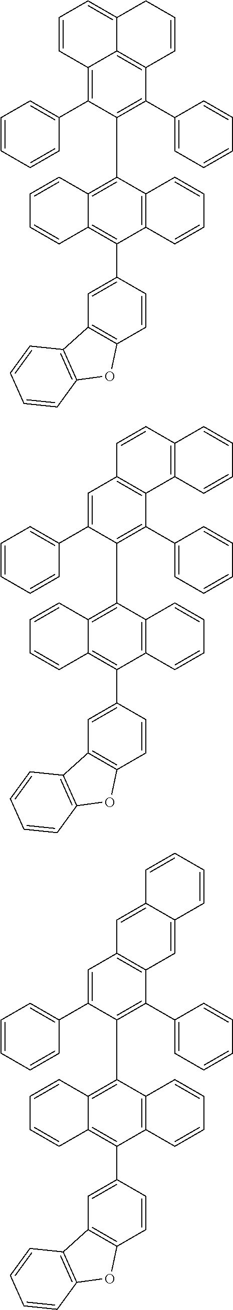 [SMILES: C1=Cc2c(-c3ccccc3)c(-c3c4ccccc4c(-c4ccc5oc6ccccc6c5c4)c4ccccc34)c(-c3ccccc3)c3cccc(c23)C1 ; c1ccc(-c2cc3ccc4ccccc4c3c(-c3ccccc3)c2-c2c3ccccc3c(-c3ccc4oc5ccccc5c4c3)c3ccccc23)cc1 ; c1ccc(-c2cc3cc4ccccc4cc3c(-c3ccccc3)c2-c2c3ccccc3c(-c3ccc4oc5ccccc5c4c3)c3ccccc23)cc1]

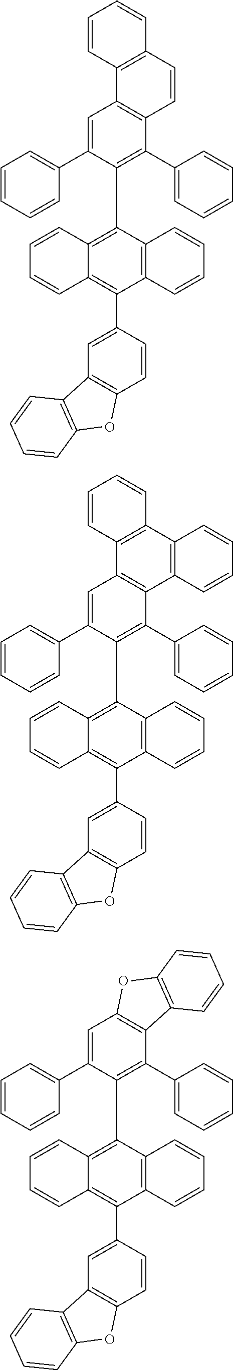

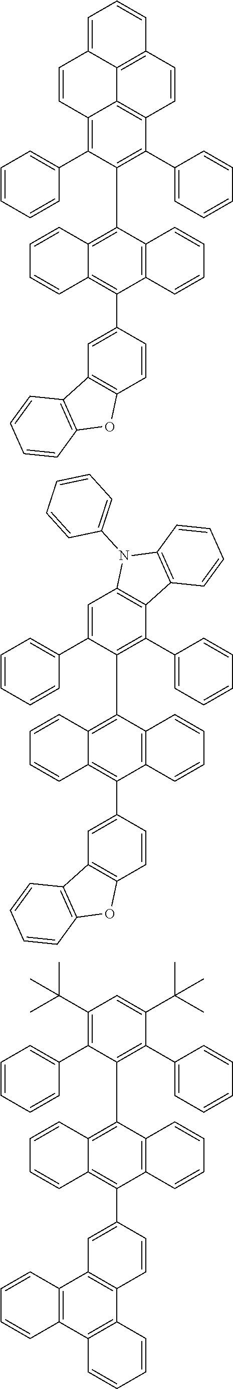

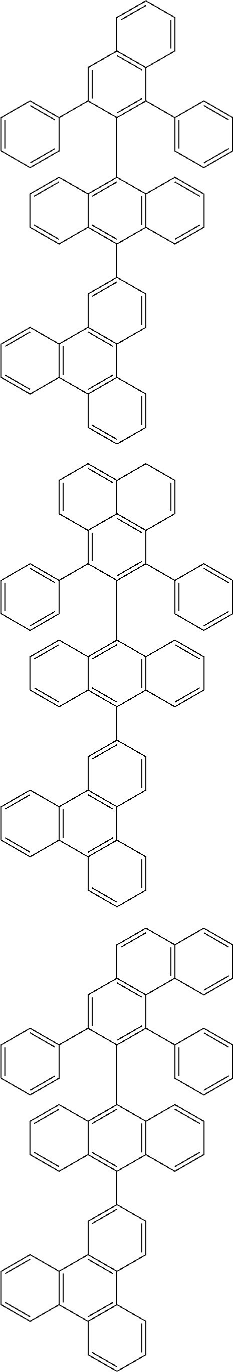

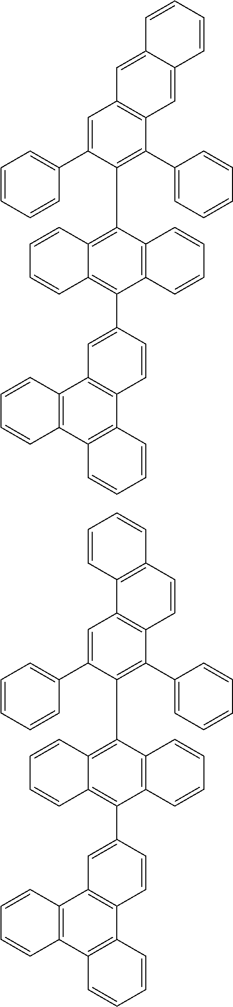

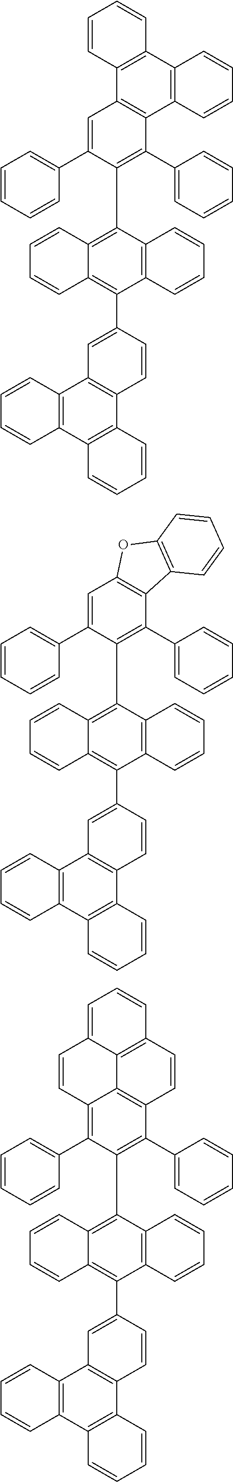

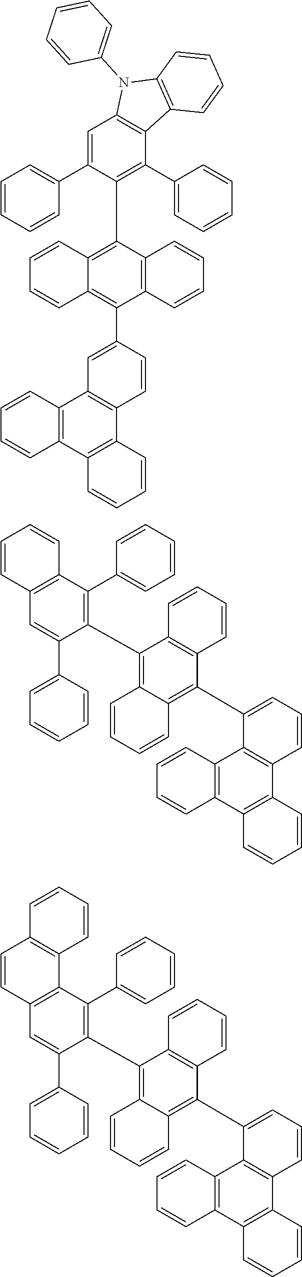

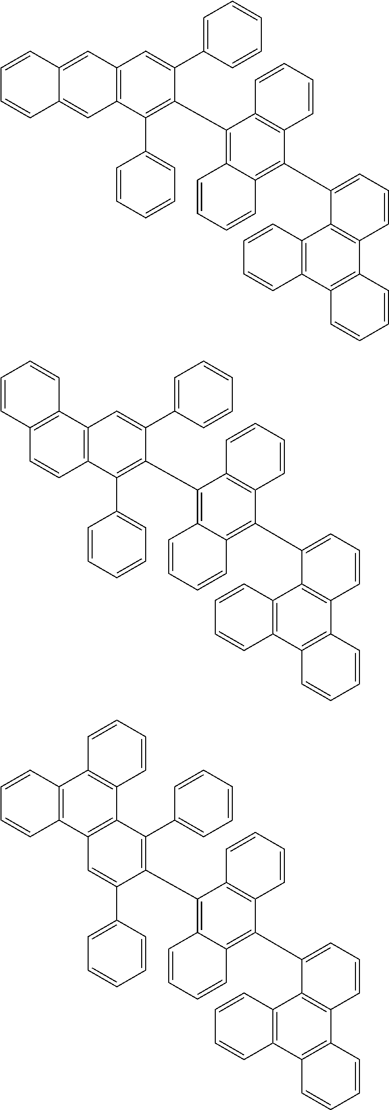

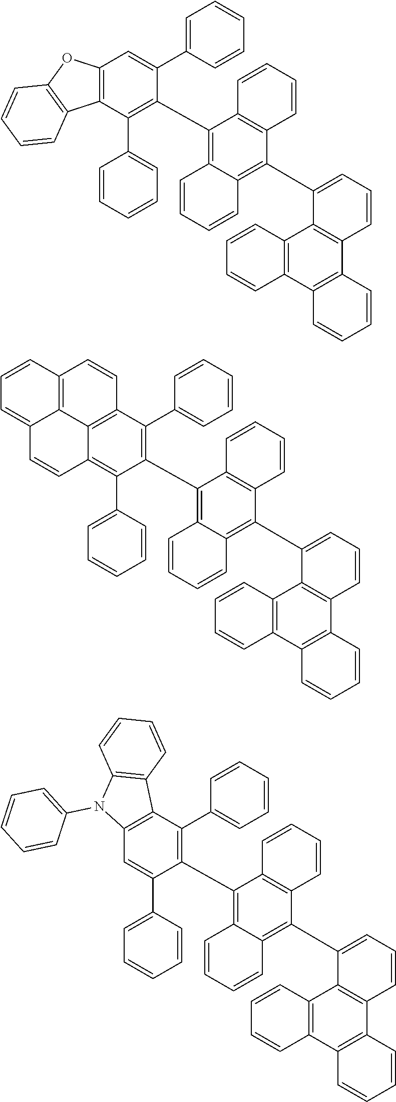

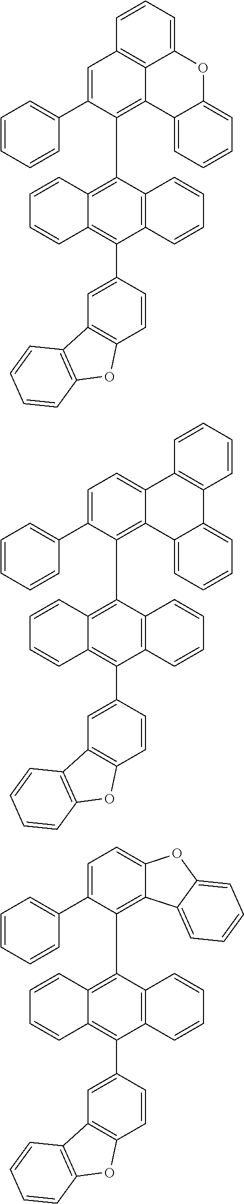

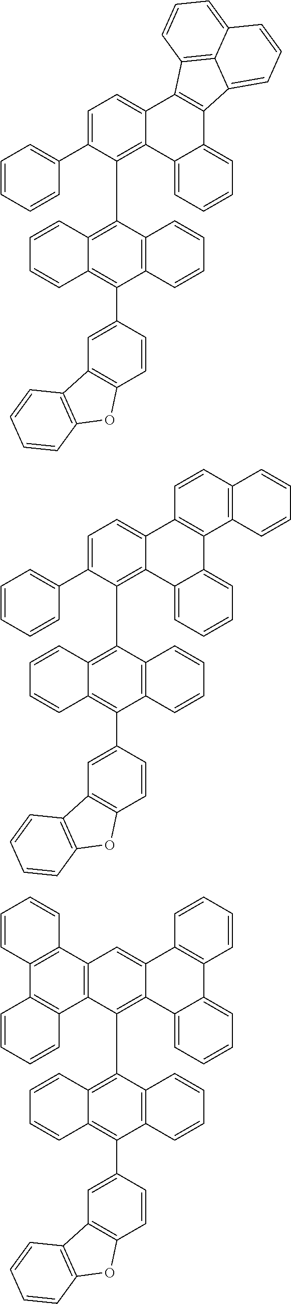

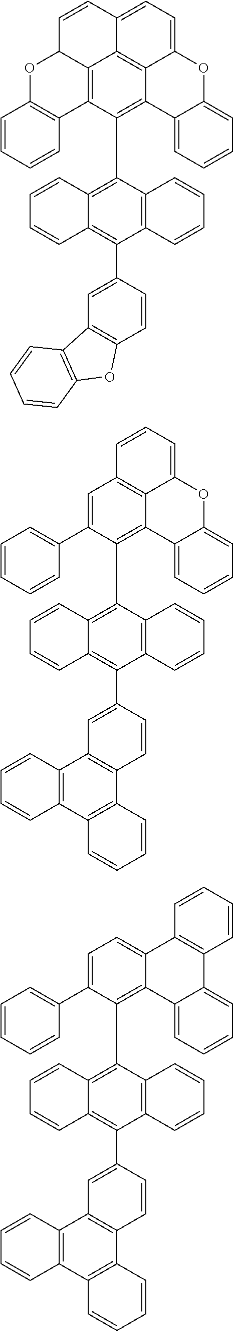

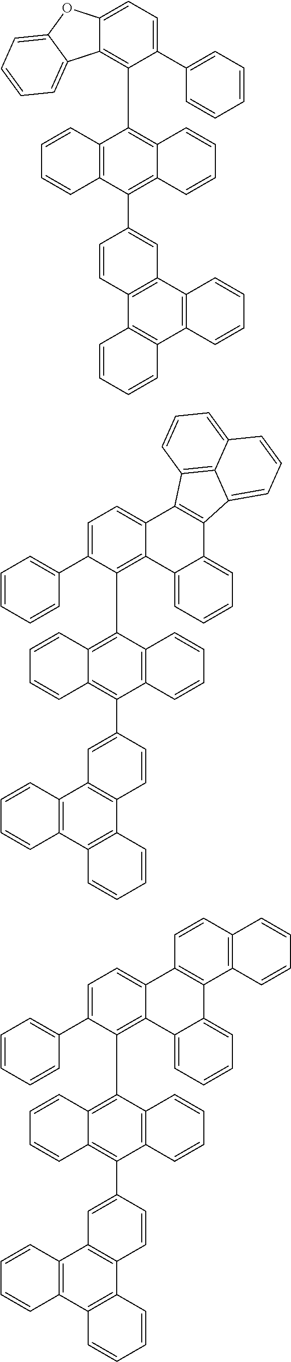

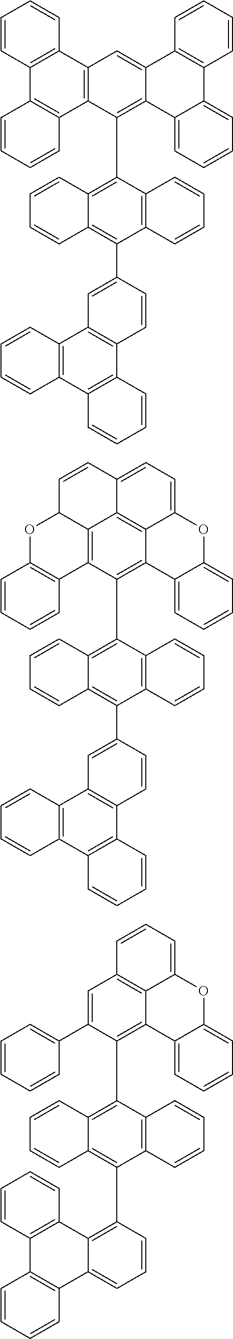

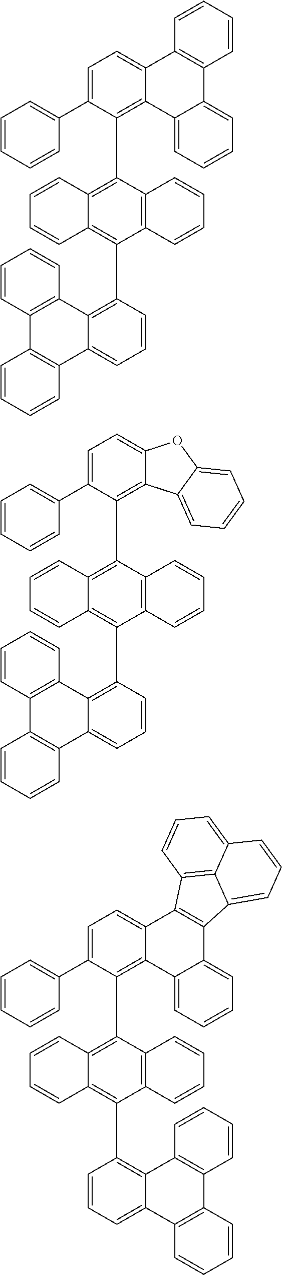

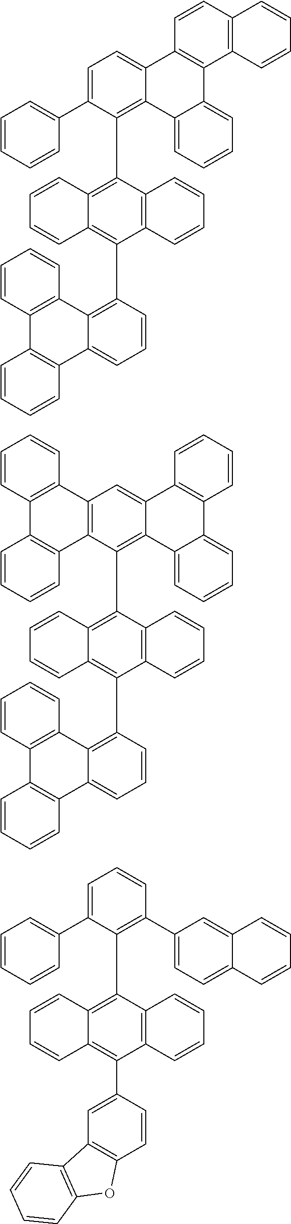

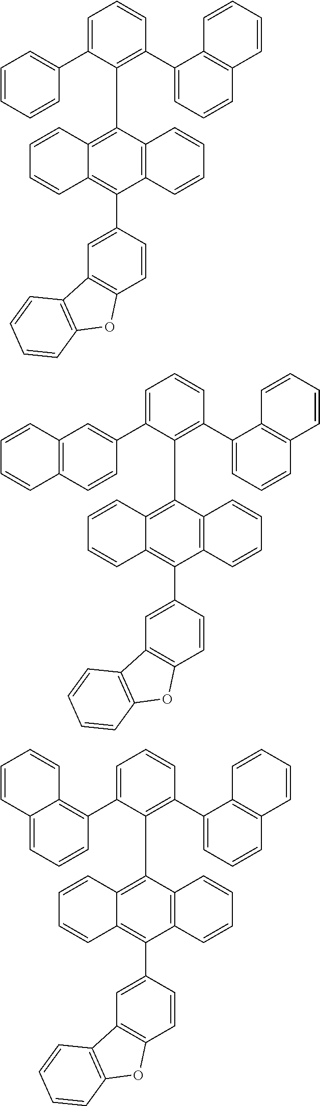

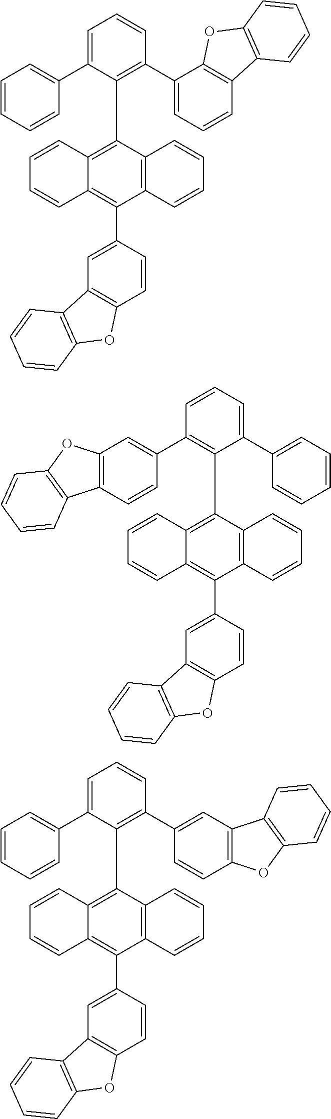

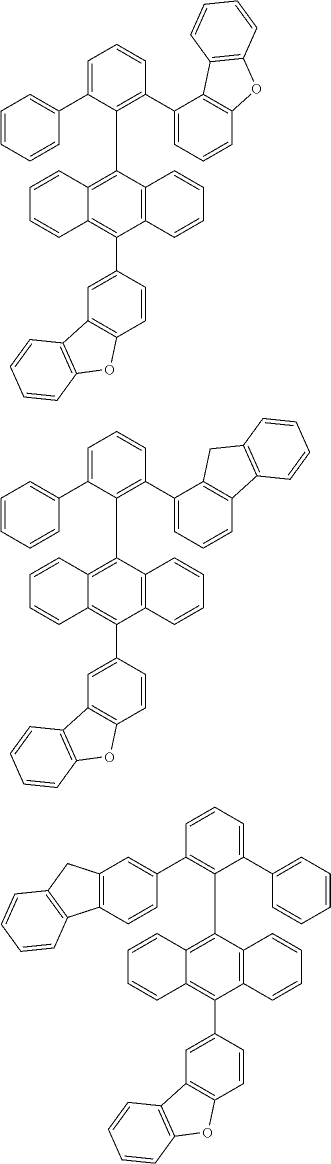 [SMILES: c1ccc(-c2cccc(-c3cccc4oc5ccccc5c34)c2-c2c3ccccc3c(-c3ccc4oc5ccccc5c4c3)c3ccccc23)cc1 ; c1ccc(-c2cccc(-c3cccc4c3Cc3ccccc3-4)c2-c2c3ccccc3c(-c3ccc4oc5ccccc5c4c3)c3ccccc23)cc1 ; c1ccc(-c2cccc(-c3ccc4c(c3)Cc3ccccc3-4)c2-c2c3ccccc3c(-c3ccc4oc5ccccc5c4c3)c3ccccc23)cc1]

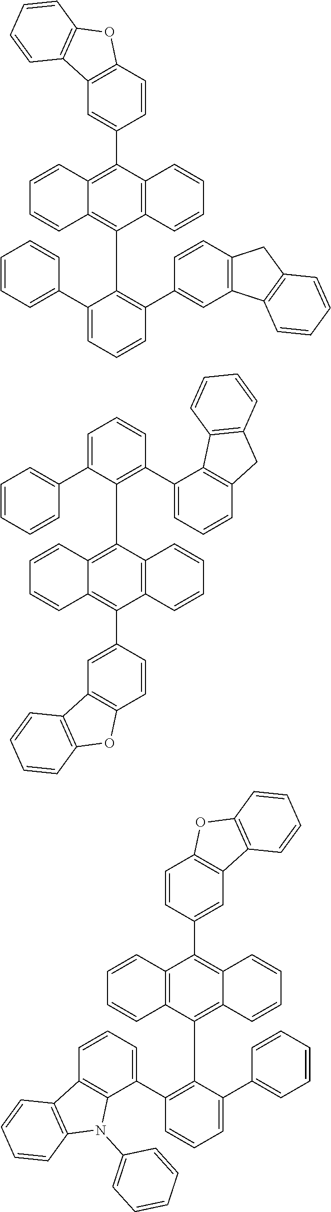 [SMILES: c1ccc(-c2cccc(-c3ccc4c(c3)-c3ccccc3C4)c2-c2c3ccccc3c(-c3ccc4oc5ccccc5c4c3)c3ccccc23)cc1 ; c1ccc(-c2cccc(-c3cccc4c3-c3ccccc3C4)c2-c2c3ccccc3c(-c3ccc4oc5ccccc5c4c3)c3ccccc23)cc1 ; c1ccc(-c2cccc(-c3cccc4c5ccccc5n(-c5ccccc5)c34)c2-c2c3ccccc3c(-c3ccc4oc5ccccc5c4c3)c3ccccc23)cc1]

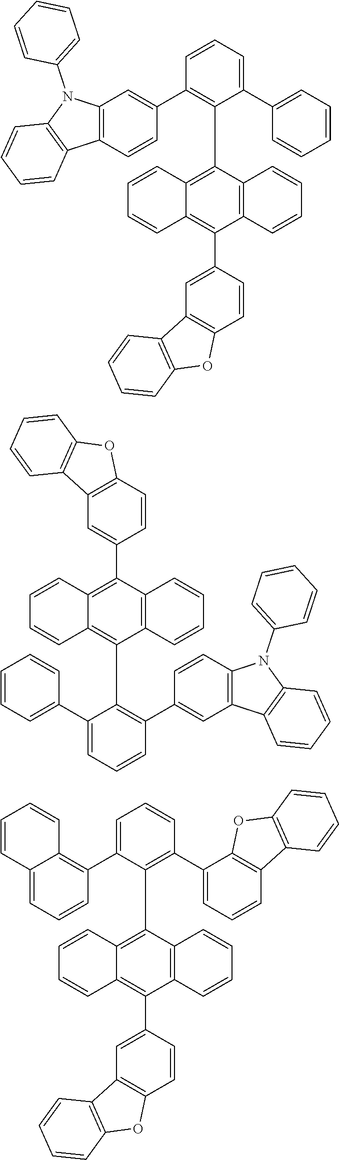

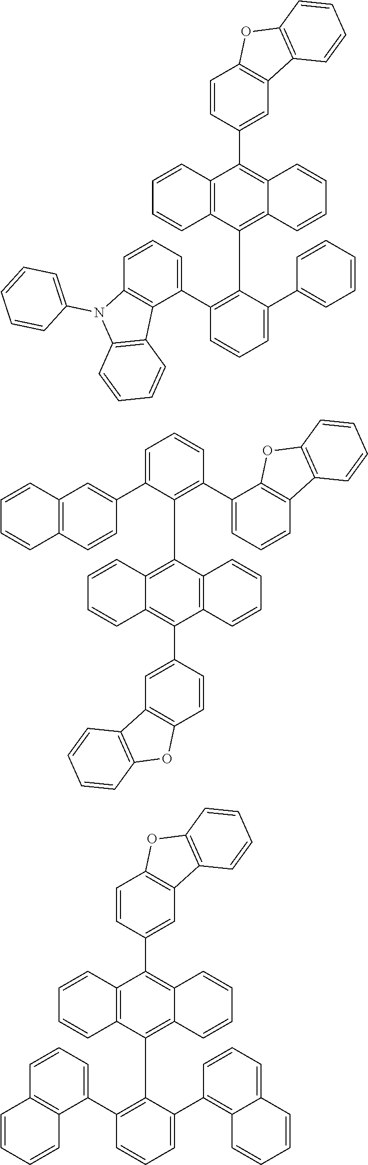

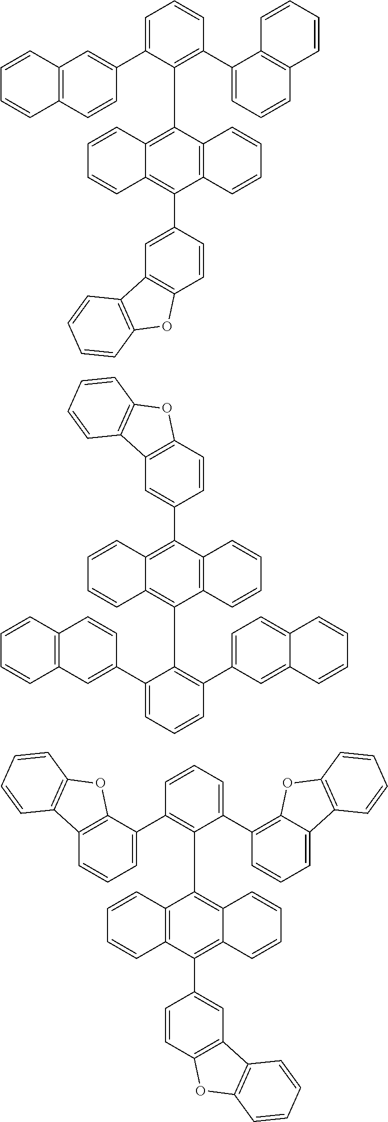

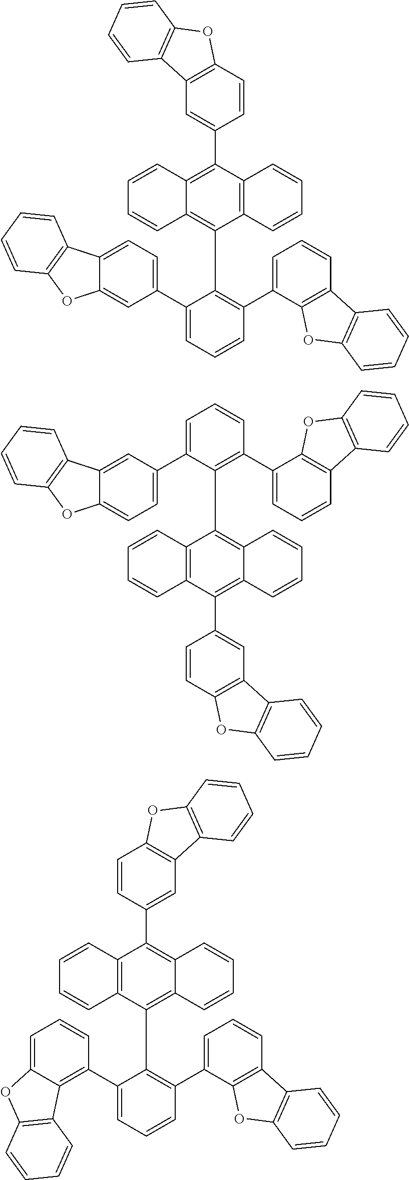

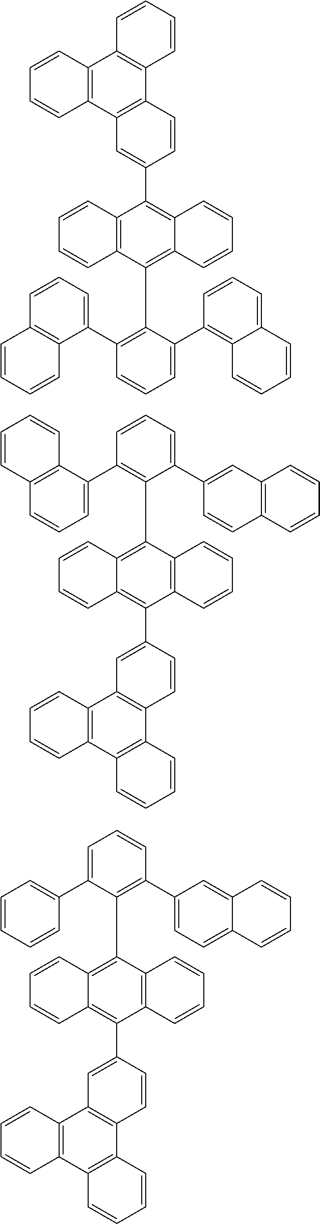

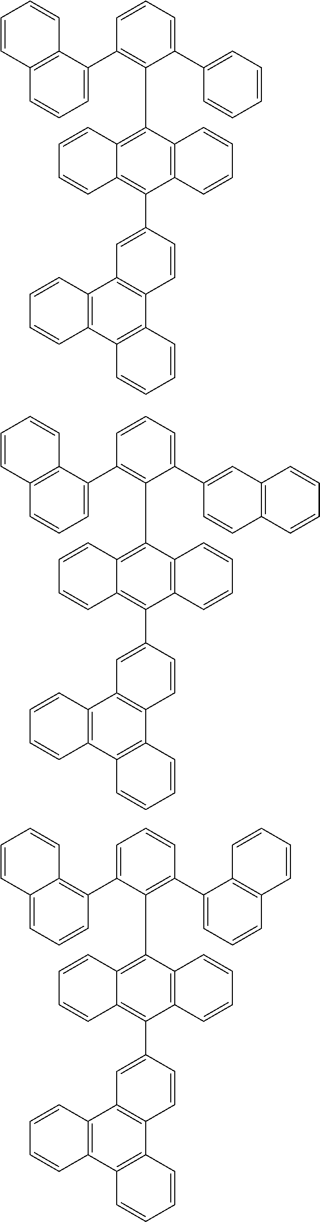

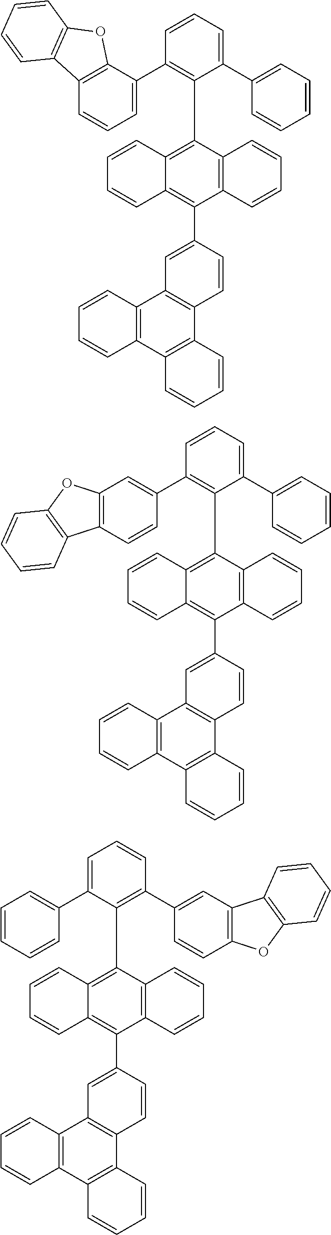

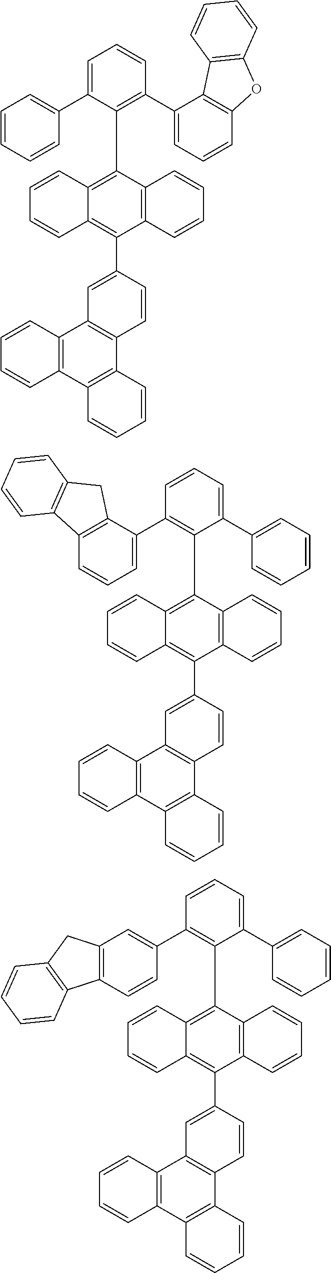

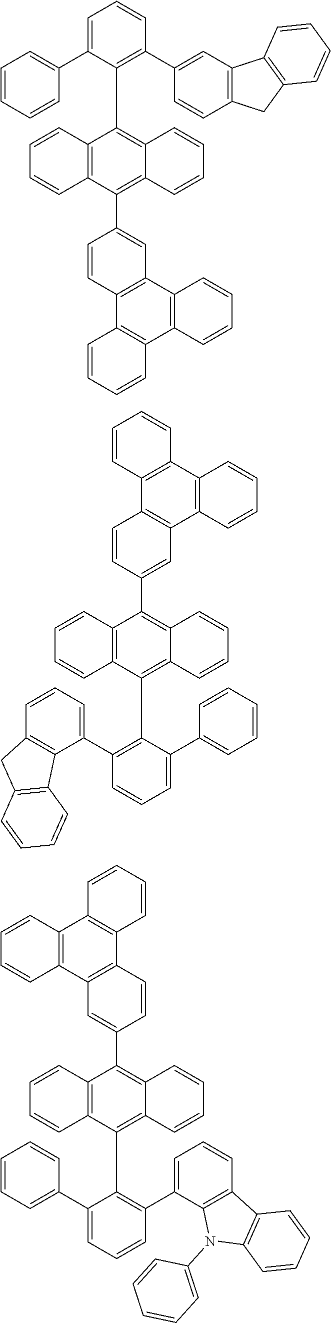

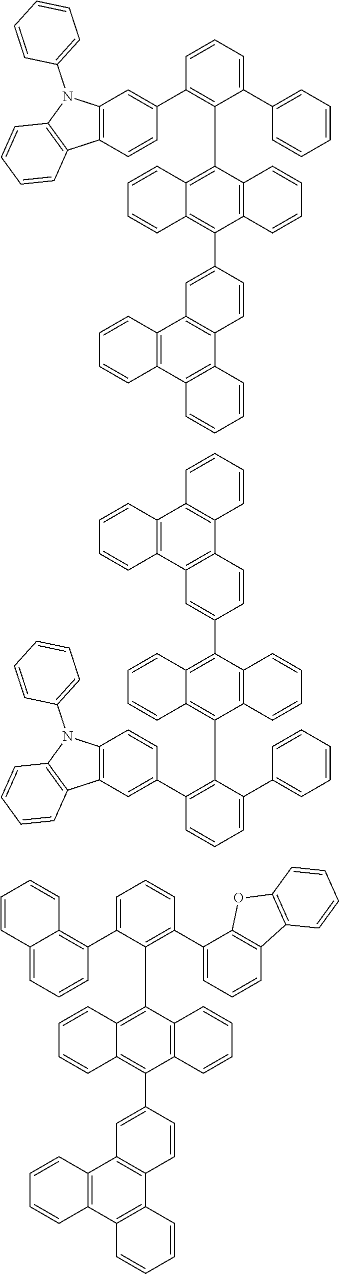

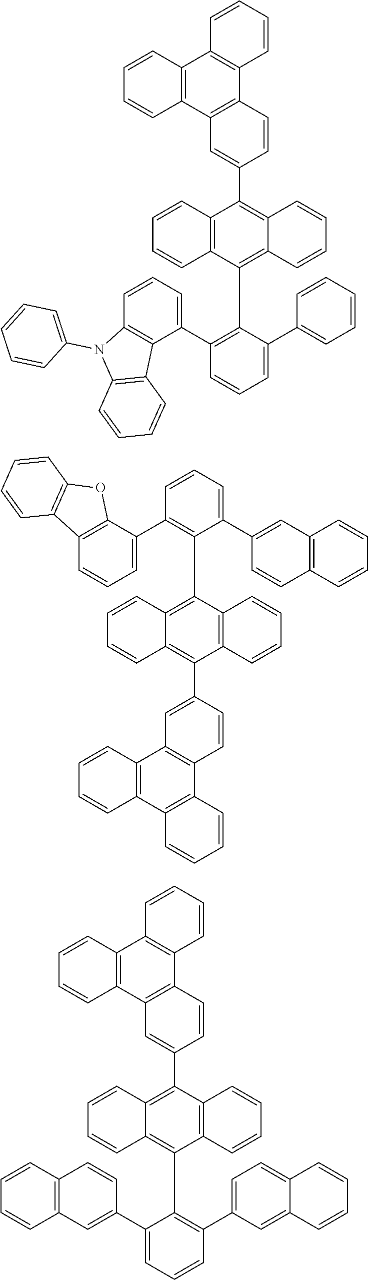

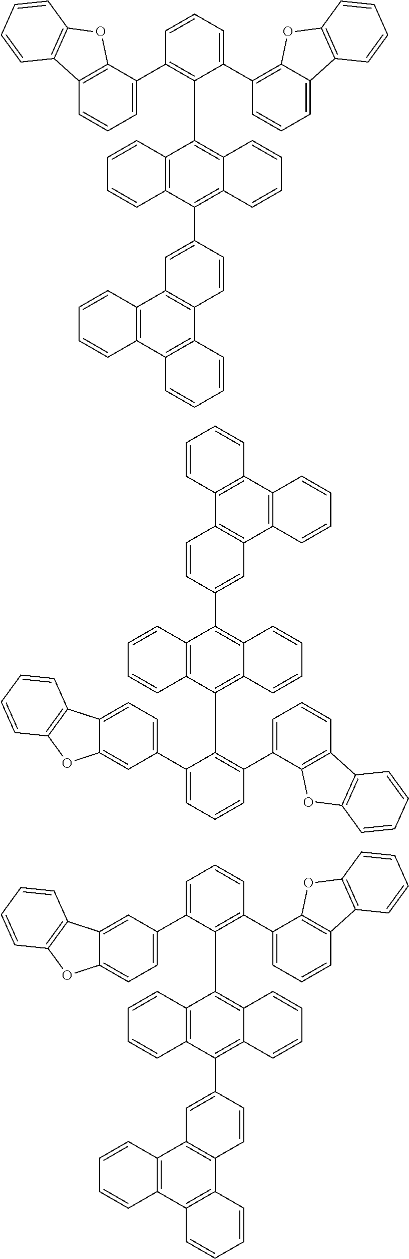

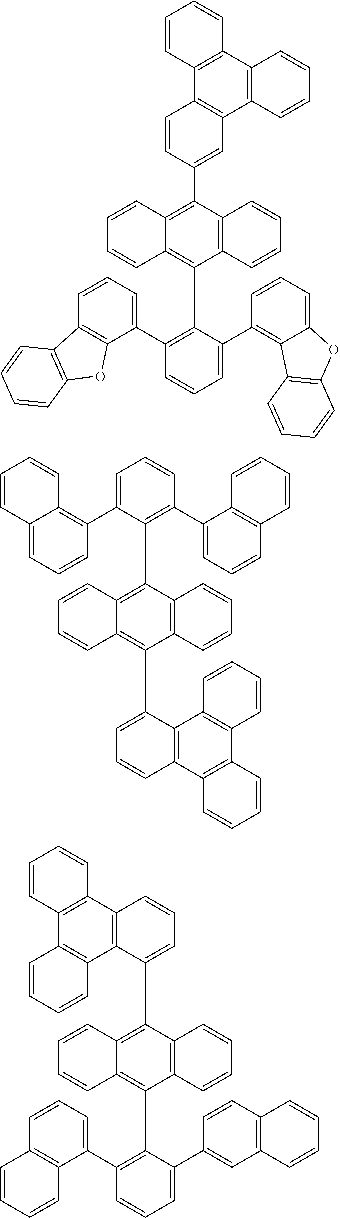

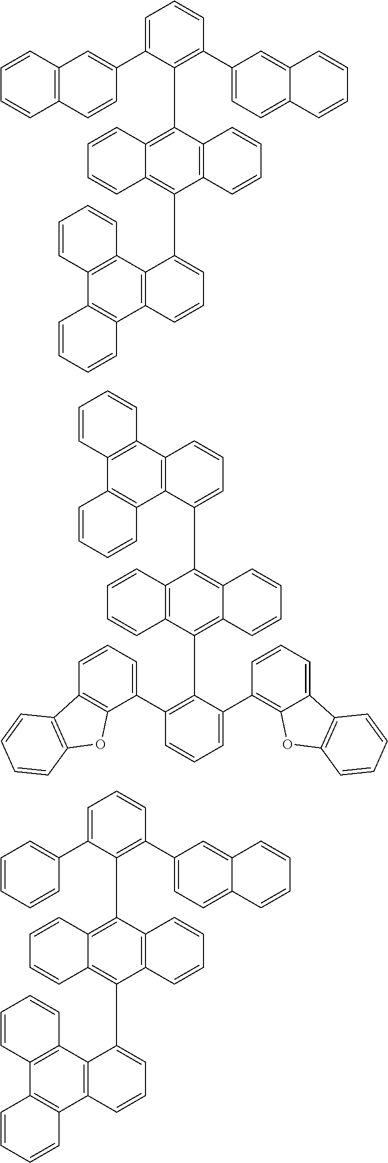

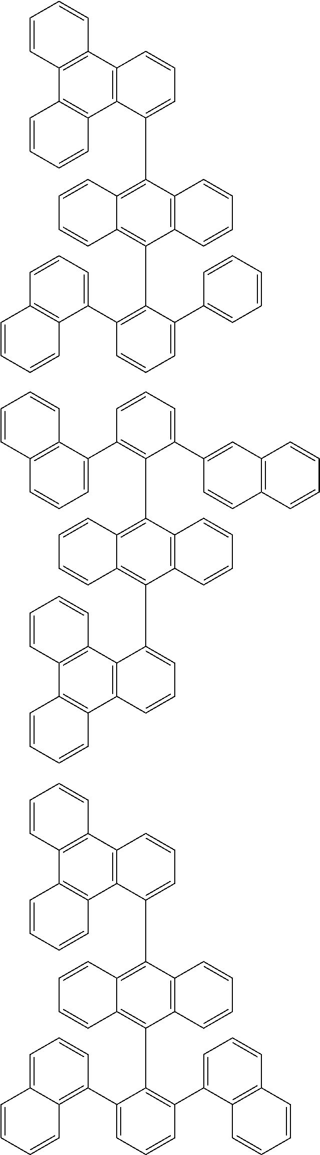

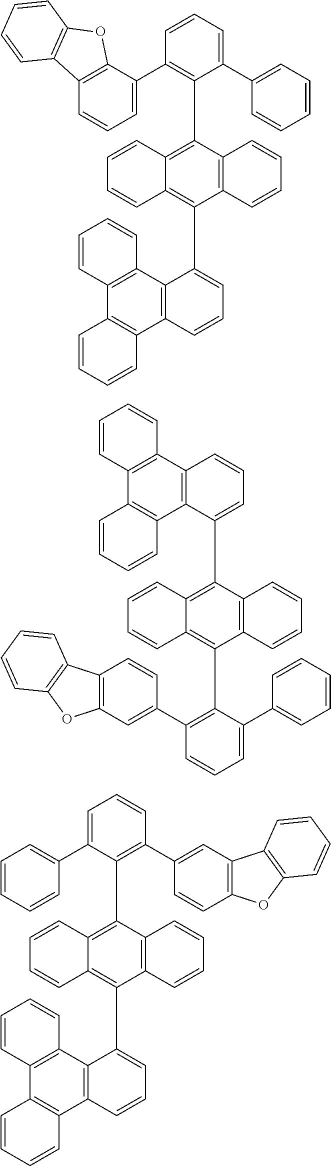

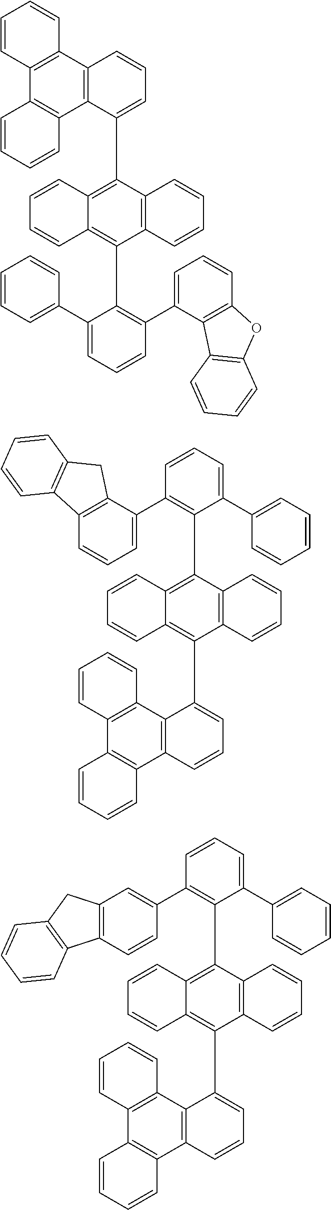

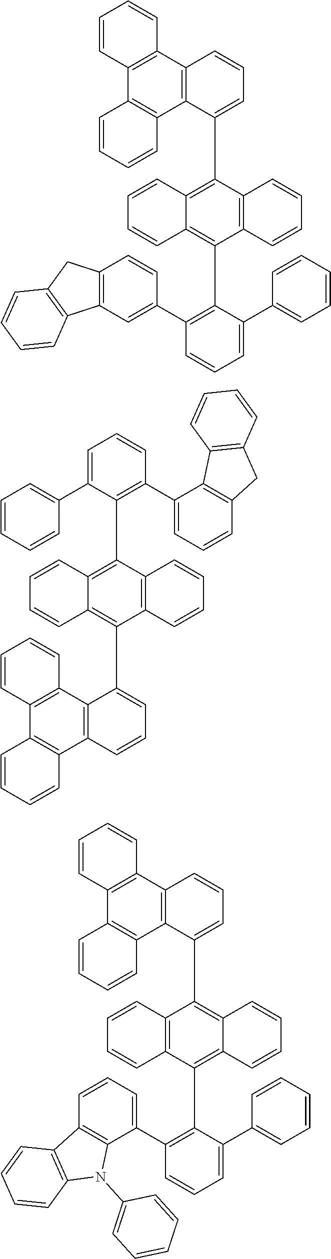

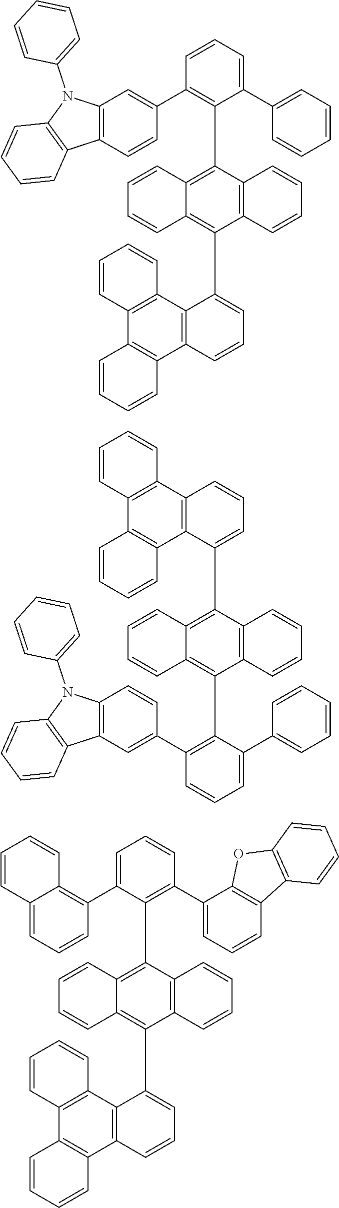

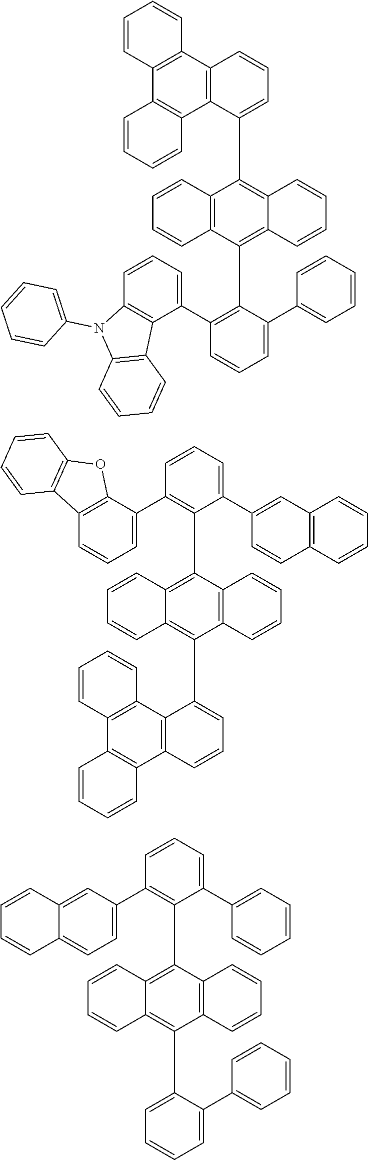

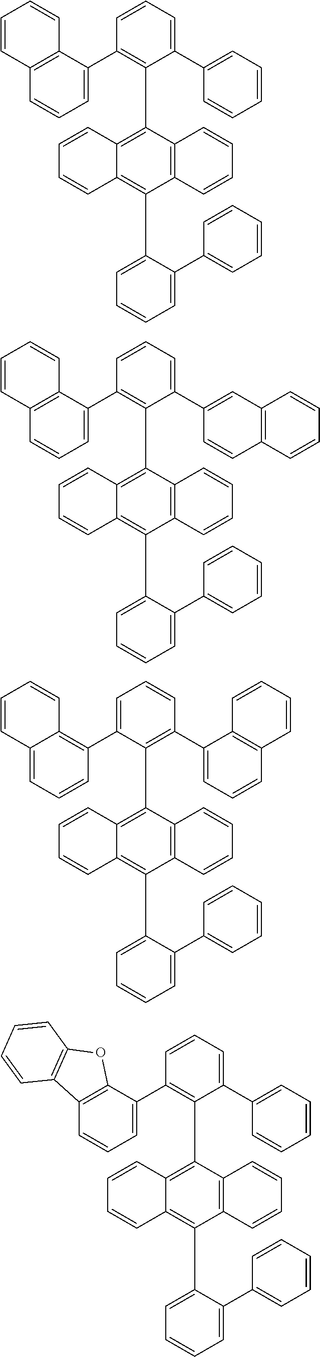

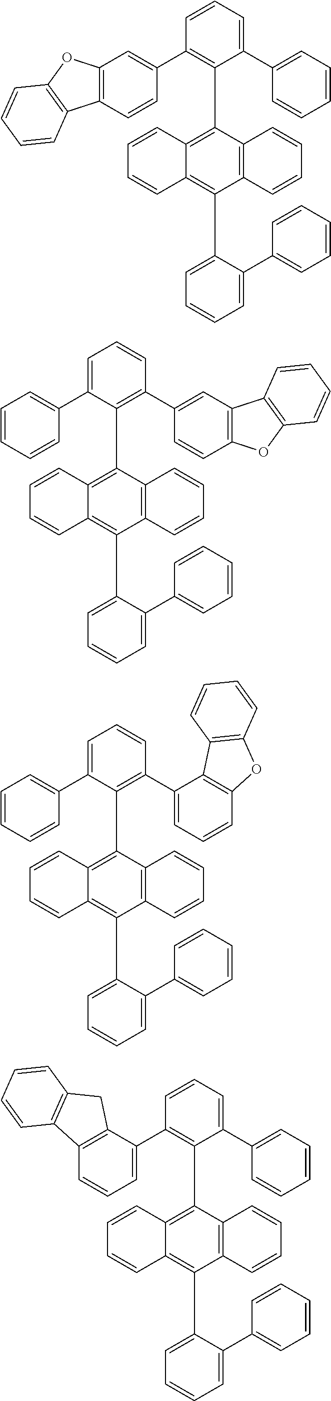

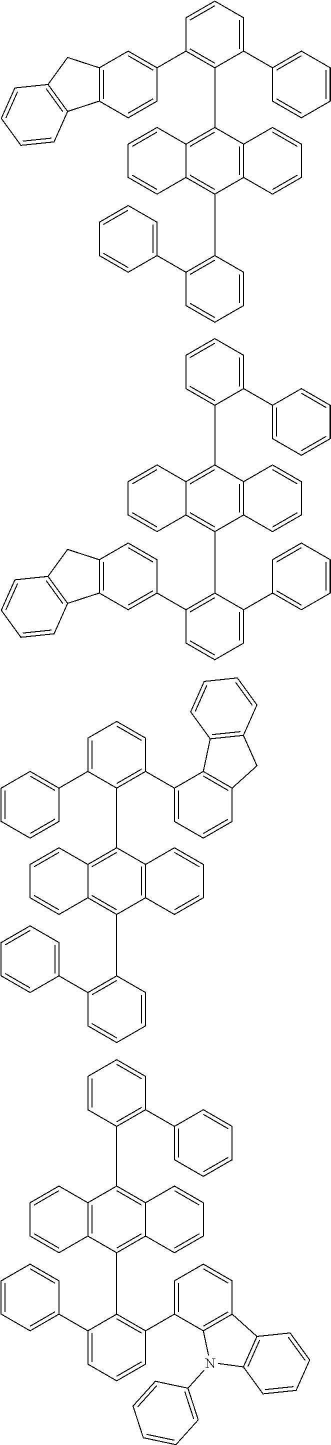

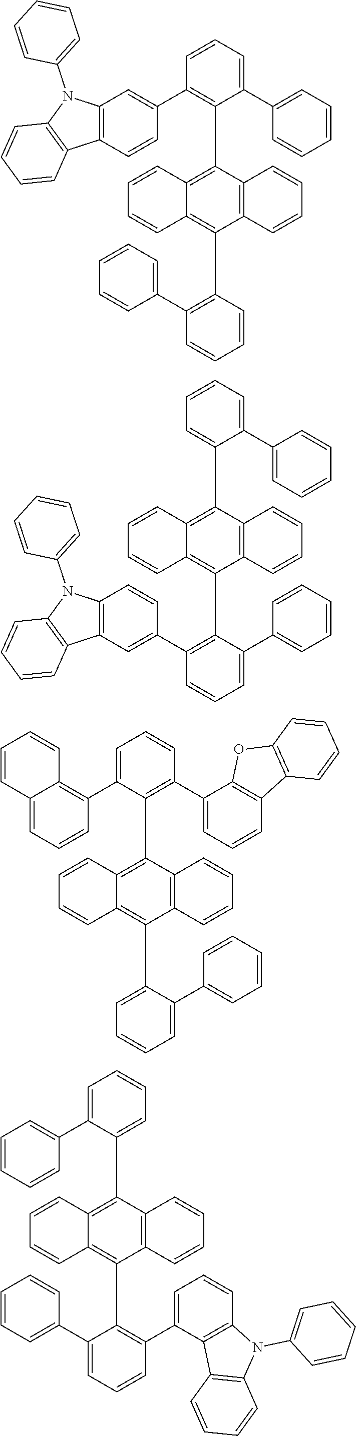

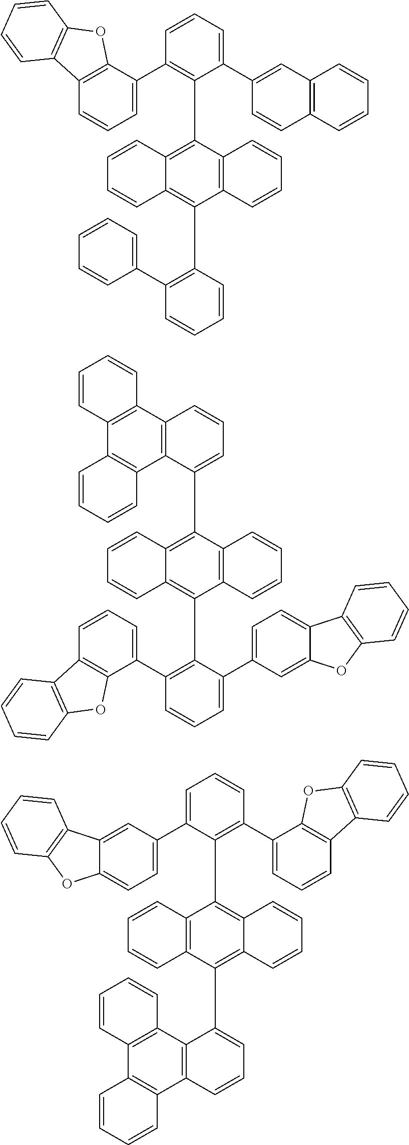 [SMILES: c1ccc(-c2ccccc2-c2c3ccccc3c(-c3c(-c4ccc5ccccc5c4)cccc3-c3cccc4c3oc3ccccc34)c3ccccc23)cc1 ; c1cc(-c2ccc3c(c2)oc2ccccc23)c(-c2c3ccccc3c(-c3cccc4c5ccccc5c5ccccc5c34)c3ccccc23)c(-c2cccc3c2oc2ccccc23)c1 ; c1cc(-c2ccc3oc4ccccc4c3c2)c(-c2c3ccccc3c(-c3cccc4c5ccccc5c5ccccc5c34)c3ccccc23)c(-c2cccc3c2oc2ccccc23)c1]

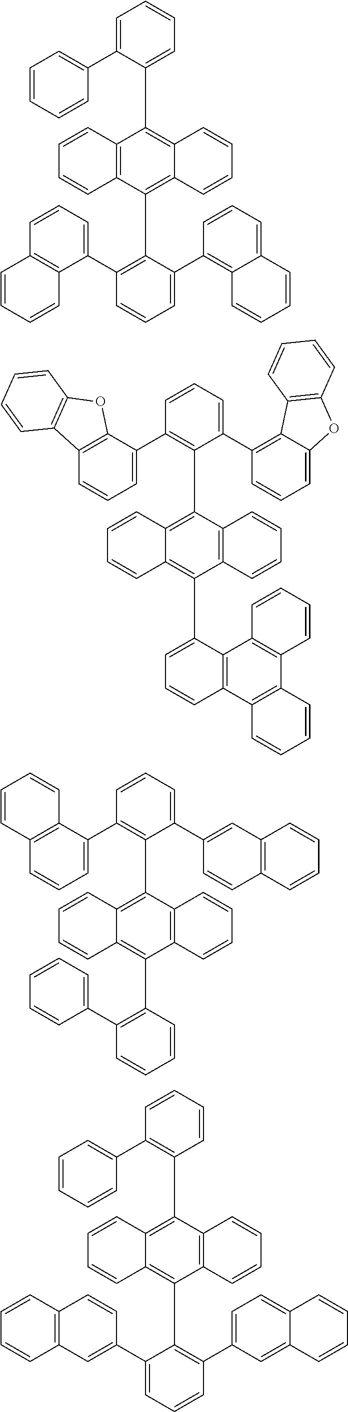

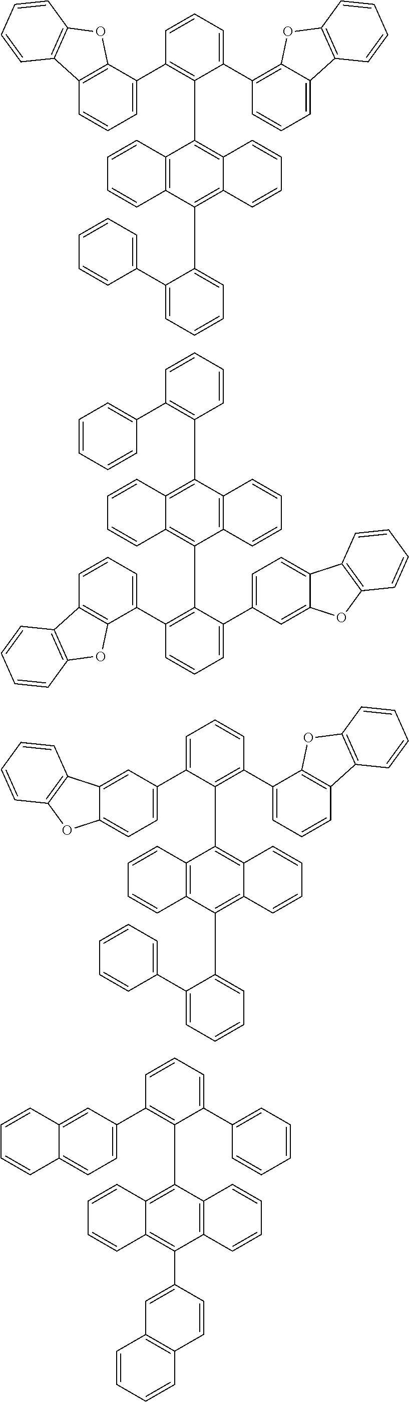

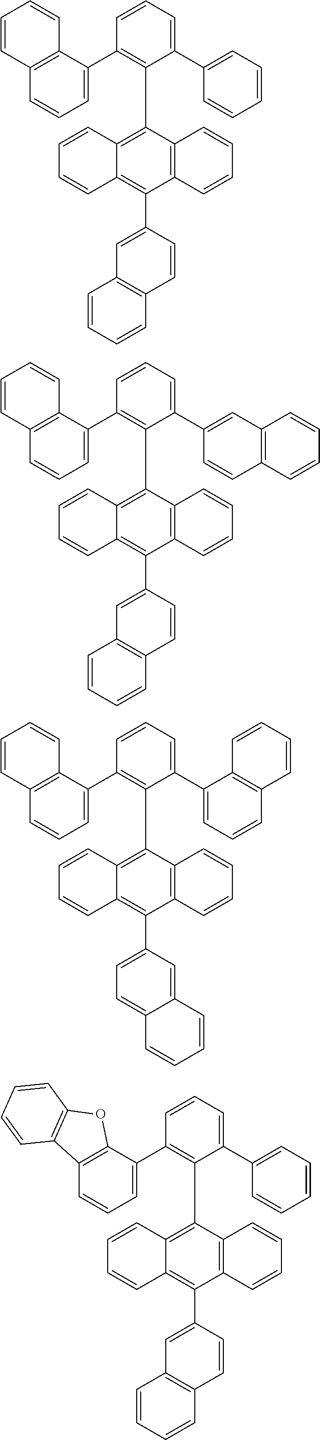

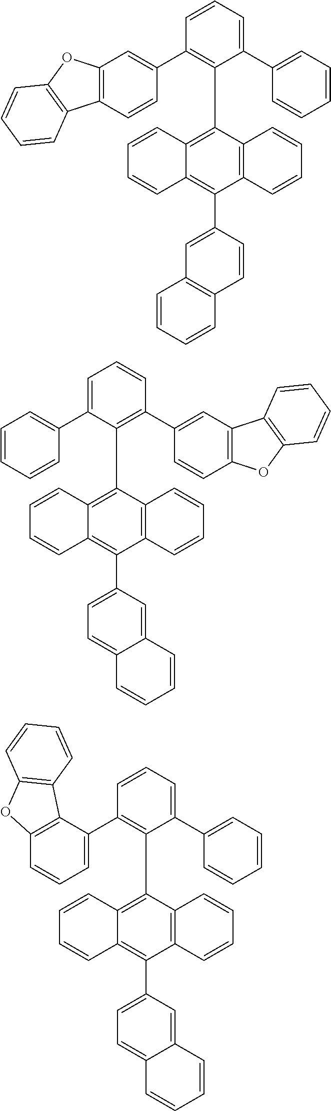 [SMILES: c1ccc(-c2cccc(-c3ccc4c(c3)oc3ccccc34)c2-c2c3ccccc3c(-c3ccc4ccccc4c3)c3ccccc23)cc1 ; c1ccc(-c2cccc(-c3ccc4oc5ccccc5c4c3)c2-c2c3ccccc3c(-c3ccc4ccccc4c3)c3ccccc23)cc1 ; c1ccc(-c2cccc(-c3cccc4oc5ccccc5c34)c2-c2c3ccccc3c(-c3ccc4ccccc4c3)c3ccccc23)cc1]

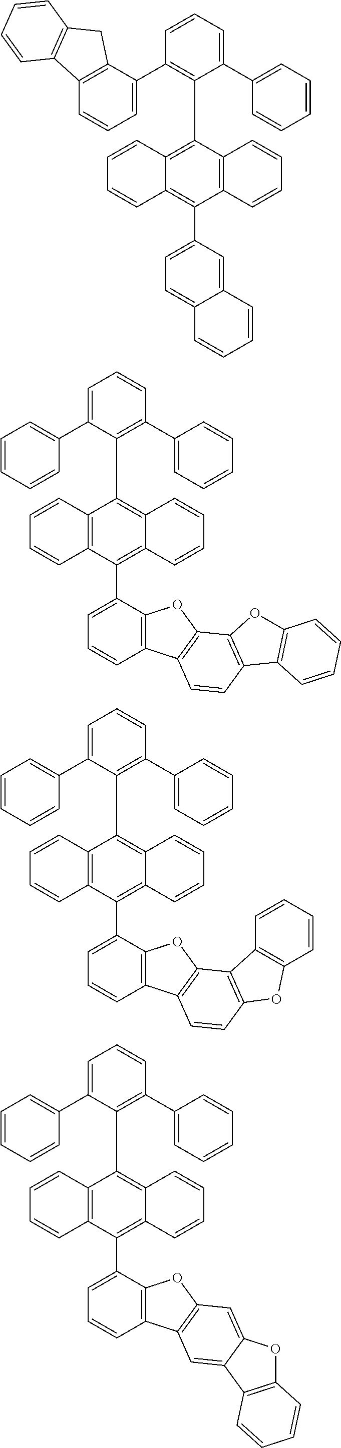

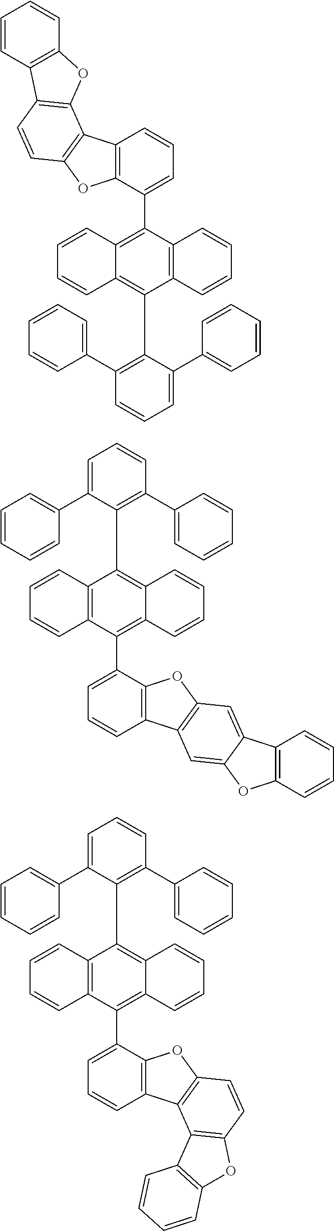

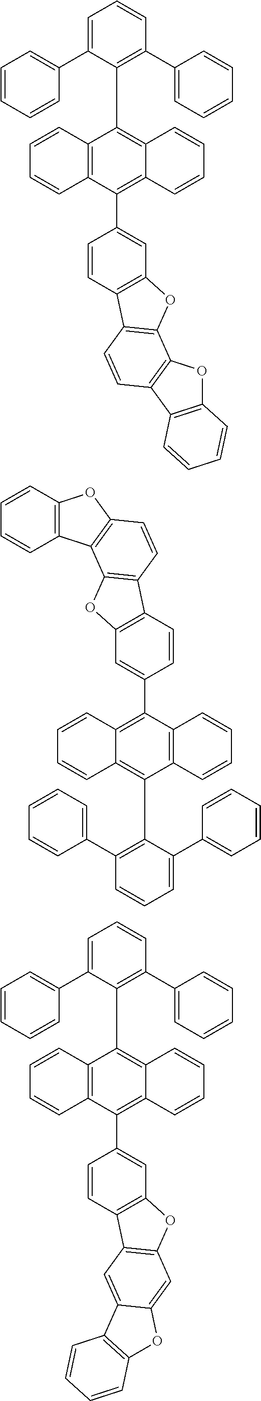

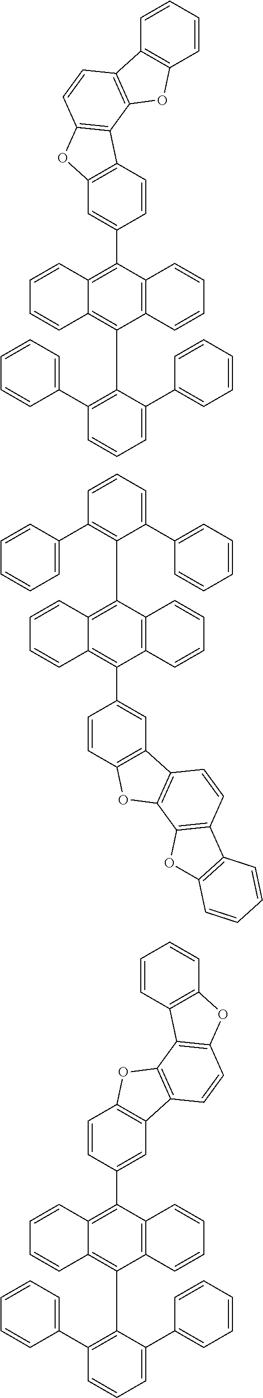

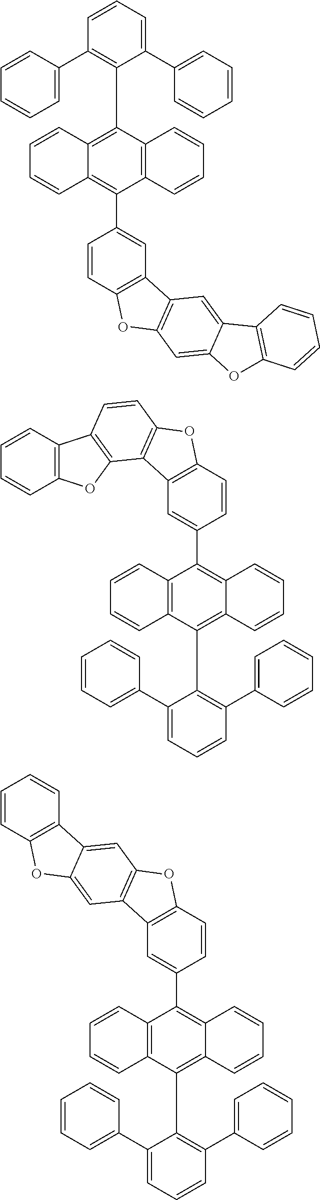 [SMILES: c1ccc(-c2cccc(-c3ccccc3)c2-c2c3ccccc3c(-c3ccc4oc5cc6oc7ccccc7c6cc5c4c3)c3ccccc23)cc1 ; c1ccc(-c2cccc(-c3ccccc3)c2-c2c3ccccc3c(-c3ccc4oc5ccc6c7ccccc7oc6c5c4c3)c3ccccc23)cc1 ; c1ccc(-c2cccc(-c3ccccc3)c2-c2c3ccccc3c(-c3ccc4oc5cc6c(cc5c4c3)oc3ccccc36)c3ccccc23)cc1]

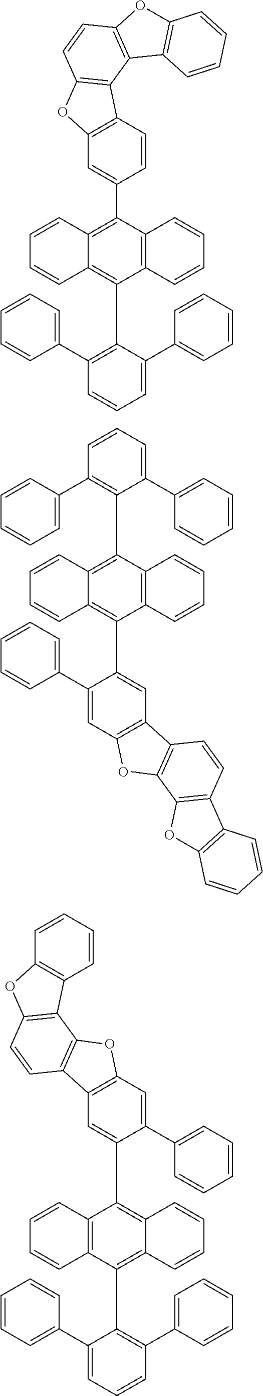

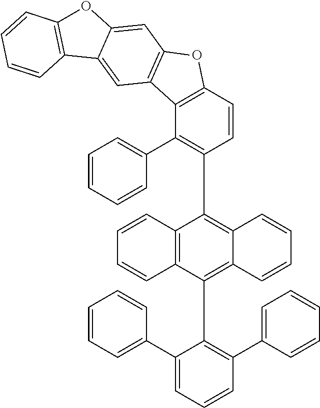 [SMILES: c1ccc(-c2cccc(-c3ccccc3)c2-c2c3ccccc3c(-c3ccc4oc5cc6oc7ccccc7c6cc5c4c3-c3ccccc3)c3ccccc23)cc1]

The organic compound as described herein can be used as an organic functional material in electronic devices, especially in OLED devices. The organic functional material is selected from a hole-injection material (HIM), a hole-transport material (HTM), an electron-transport material (ETM), an electron-injection material (EIM), an electron-blocking material (EBM), a hole-blocking material (HBM), an emitting material (Emitter), a host material (Host), or an organic dye.

In some embodiments, the organic compound as described herein can be used as a fluorescent host material or a co-host material.

As the phosphorescent host material, the organic compound should have an appropriate triplet state energy level, namely T₁. In some embodiments, the organic compound as described herein, its T₁≤2.0 eV, preferably ≤1.9 eV, and most preferably ≤1.8 eV.

As the fluorescent host material, the organic compound should have an appropriate singlet energy level, namely S₁. In some embodiments, the organic compound as described herein, its S₁≥2.8 eV, preferably ≥2.85 eV, more preferably ≥2.9 eV, and most preferably ≥2.95 eV.

In some embodiments, the organic compound as described herein, its (T₂−S₁)≤0.2 eV, preferably ≤0.15 eV, more preferably ≤0.1 eV, further preferably ≤0.05 eV, and most preferably ≈0 eV. Here T₂ stands for the second triplet state energy level.

In some embodiments, the organic compound as described herein, its (2T₁−S₁)≥0.1 eV, preferably ≥0.15 eV, more preferably ≥0.2 eV, further preferably ≥0.25 eV, and most preferably ≥0.3 eV.

As a fluorescent host material, it is desirable to have good thermal stability. Generally, the glass transition temperature (Tg) of the organic compound≥100° C.; in some embodiments, Tg≥120° C.; in some embodiments, Tg≥140° C.; in some embodiments, Tg≥160° C.

In some embodiments, the (HOMO−(HOMO−1)) of the organic compound≥0.2 eV, preferably ≥0.25 eV, more preferably ≥0.3 eV, even more preferably ≥0.35 eV, further preferably ≥0.4 eV, and most preferably ≥0.45 eV.

In some embodiments, the ((LUMO+1)−LUMO) of the organic compound≥0.15 eV, preferably ≥0.20 eV, more preferably ≥0.25 eV, further preferably ≥0.30 eV, and most preferably ≥0.35 eV.

In some embodiments, the organic compound as described herein has a light-emitting function with an emitting wavelength between 300 nm and 1000 nm, preferably between 350 nm and 900 nm, more preferably between 400 nm and 800 nm. Herein the emission refers to photoluminescence or electroluminescence.

In another aspect, the present disclosure also provides to a polymer comprising at least one repeating unit, the at least one repeating unit comprises a structure of formula (I).

In some embodiments, the polymer is a non-conjugated polymer in which the structural unit of formula (I) is on a side chain. In some embodiments, the polymer is a conjugated polymer.

The term “small molecule” herein refers to a molecule that is no one of following: a polymer, an oligomer, a dendrimer, or a blend. In particular, there are no repeating structures in the small molecule. The molecular weight of the small molecule≤4000 g/mol, preferably ≤3000 g/mol, and most preferably ≤2000 g/mol.

The term “polymer” comprises homopolymer, copolymer, and block copolymer. Also in the present disclosure, the term “polymer” comprises dendrimer. For the synthesis and application of the dendrimers please refer to [Dendrimers and Dendrons, Wiley-VCH Verlag GmbH & Co. KGaA, 2002, Ed. George R. Newkome, Charles N. Moorefield, Fritz Vogtle.].

The term “conjugated polymer” refers to a polymer with backbone mainly comprising sp² hybrid orbitals of C atoms, well-known examples are polyacetylene and poly(phenylene vinylene). The C atoms on the backbones can also be substituted with other non-C atoms. Moreover, the above-mentioned structure should still be considered as a conjugated polymer when the sp² hybridization on the backbone is interrupted by natural defects. Also in the present disclosure, the backbone of conjugated polymer comprises aryl amines, aryl phosphines and other heteroarmotics, organometallic complexes, etc.

In some embodiments, the synthetic method of the polymer is selected from the group consisting of SUZUKI-, YAMAMOTO-, STILLE-, NIGESHI-, KUMADA-, HECK-, SONOGASHIRA-, HIYAMA-, FUKUYAMA-, HARTWIG-BUCHWALD-, and ULLMAN-.

In some embodiments, the glass transition temperature (Tg) of the polymer≥100° C., preferably ≥120° C., more preferably ≥140° C., further preferably ≥160° C., and most preferably ≥180° C.

In some embodiments, the polydispersity index (PDI) of the polymer is preferably from 1 to 5, more preferably 1 to 4; even more preferably 1 to 3, further preferably 1 to 2, and most preferably 1 to 1.5.

In some embodiments, the weight-average molecular weight (M_{w}) of the polymer is preferably from 10 k to 1 million, more preferably 50 k to 500 k, even more preferably 100 k to 400 k, further preferably 150 k to 300 k, and most preferably 200 k to 250 k.

In yet another aspect, the present disclosure further provides a mixture comprising an organic compound or a polymer as described above, and at least one organic functional material. The at least one organic functional material is selected from a hole-injection material (HIM), a hole-transport material (HTM), a hole-blocking material (HBM), an electron-injection material (EIM), an electron-transport material (ETM), an electron-blocking material (EBM), an emitting material, a host material (Host), a singlet emitting material (fluorescent emitting material), a triplet emitting material (phosphorescent emitting material), a thermally activated delayed fluorescence material (TADF material), especially is a luminescent organometallic complex. These organic functional materials are described in detail, for example, in WO2010135519A1, US20090134784A1, and WO2011110277A1. The entire contents of these three documents are hereby incorporated into this document for reference. The at least one organic functional material can be a small molecule and polymer material.

In some embodiments, the mixture comprises an organic compound or a polymer as described above, and a fluorescent emitting material. The organic compound or polymer as described herein can be used here as fluorescent host material with the weight percentage≤10 wt %, preferably ≤9 wt %, more preferably ≤8 wt %, particularly preferably ≤7 wt %, and most preferably ≤5 wt %.

In some embodiments, the mixture comprises an organic compound or a polymer as described above, and a HTM material.

In some embodiments, the mixture comprises an organic compound as described above, and a fluorescent emitting material (singlet emitting material). The organic compound herein may be used as a host in a weight ratio of 80% to 95%, preferably 85% to 95%, preferably 90% to 95%.

In some embodiments, the mixture comprises an organic compound as described above, and another singlet host material. The organic compound herein can be used as a co-host, and the weight ratio of which to the another singlet host material is from 1:5 to 5:1, preferably from 1:4 to 4:1, more preferably from 1:3 to 3:1, and most preferably from 1:2 to 2:1.

In some embodiments, the mixture comprises an organic compound as described above, a singlet host material, and a fluorescent emitting material (singlet emitting material). The organic compound herein can be used as a co-host.

The singlet host and fluorescent emitting material are described in detail below (but not limited thereto).

### Singlet Host

Examples of singlet host materials are not specially limited, any organic compound may be used as a host, as long as its singlet energy is higher than emitter, especially higher than that of the singlet emitter or the fluorescent emitter.

Examples of the organic compound used as the singlet host material may be selected from cyclic aromatic hydrocarbon compound, such as benzene, biphenyl, triphenylbenzene, benzophenanthrene, triphenylene, anthracene, phenalene, phenanthrene, fluorene, pyrene, chrysene, perylene, azulene. The organic compound used as the singlet host material may be also selected from aromatic heterocyclic compound, such as dibenzothiophene, dibenzofuran, dibenzothiophene, furan, thiophene, benzofuran, benzothiophene, benzoselenophene, carbazole, indocarbazole, pyridindole, pyrroledipyridine, pyrazole, imidazole, triazole, isoxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indolizine, benzoxazole, benzoisoxazole benzothiazole, quinoline, isoquinoline, cinnoline, quinazoline, quinoxaline, naphthalene, phthalein pteridine, xanthene, acridine, phenazine, phenothiazine, phenoxazine, benzofuranopyridine, furandipyridine, benzothienopyridine, thiophenedipyridine, benzoselenophenopyridine, or selenophenodipyridine. The organic compound used as the singlet host material may be selected from groups containing 2 to 10 ring atoms, which may be the same or different types of cyclic aryl or heterocyclic aryl, and are linked to each other directly or by at least one of the following groups, such as oxygen atom, nitrogen atom, sulfur atom, silicon atom, phosphorus atom, boron atom, chain structure unit, or aliphatic ring group.

In some embodiments, the singlet host material may be selected from the compounds comprising at least one of the following groups:

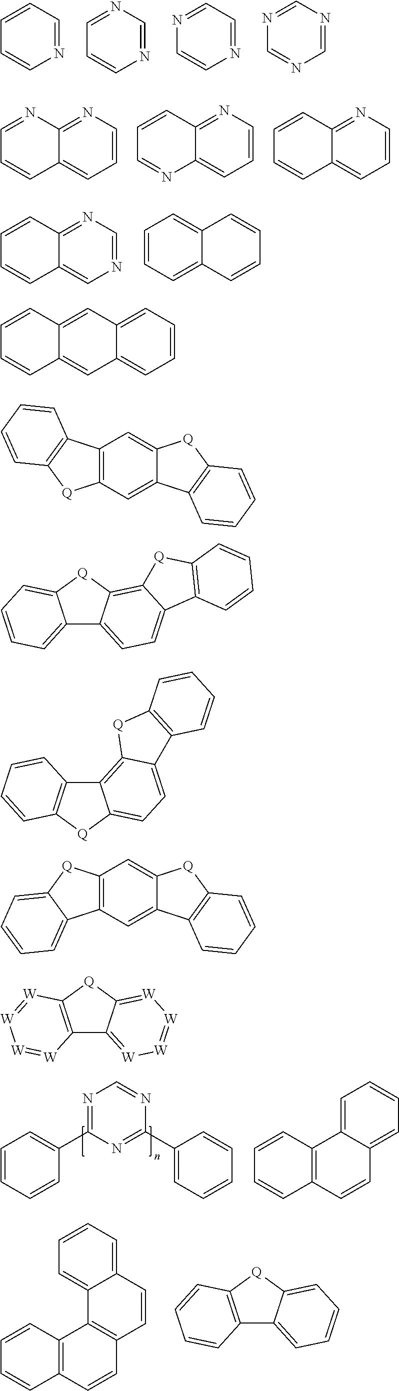

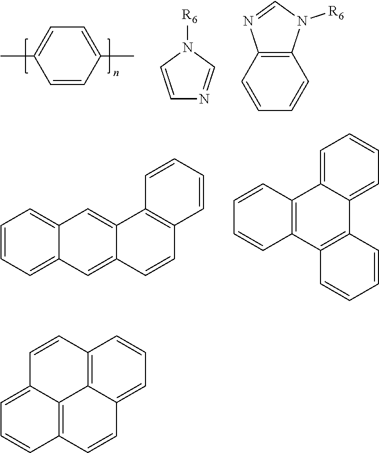

Q at each occurrence is independently selected from C(R)², NR, O, or S; W at each occurrence is CR or R; R₆ at each occurrence is independently selected from the following groups: H, deuterium, halogen atom (F, Cl, Br, I), cyano, alkyl, alkoxy, amino, alkenyl, alkynyl, aralkyl, heteroalkyl, aryl, and heteroaryl, and each n is an integer from 1 to 20.

In some embodiments, the singlet host is selected from anthracene derivatives, such examples are disclosed in the patent literature such as CN102224614B, CN100471827C, CN1914293B, WO2015033559A1, US2014246657A1, WO2016117848A1, WO2016117861A1, WO2016171429A2, CN102369256B, CN102428158B, etc.

The following are some examples of anthracene-based singlet host materials:

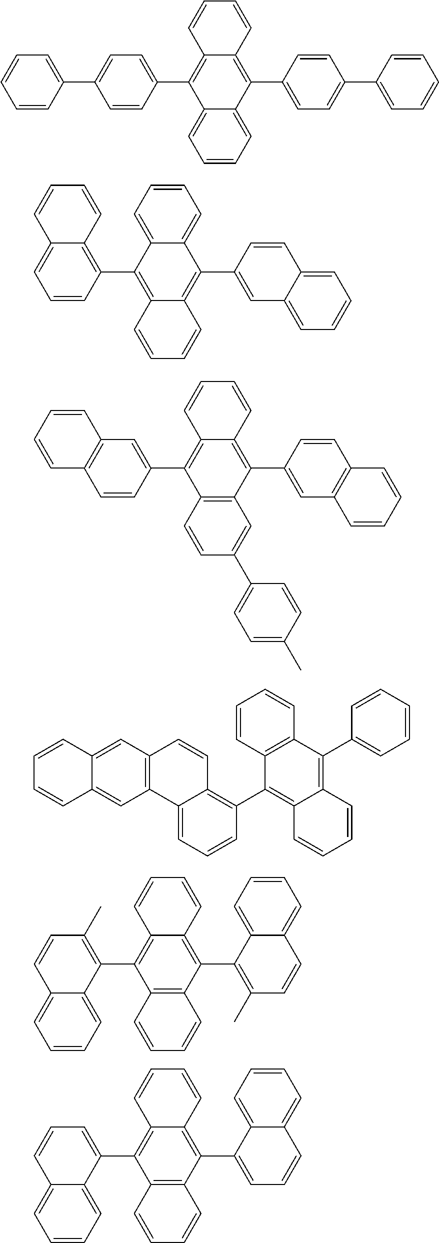

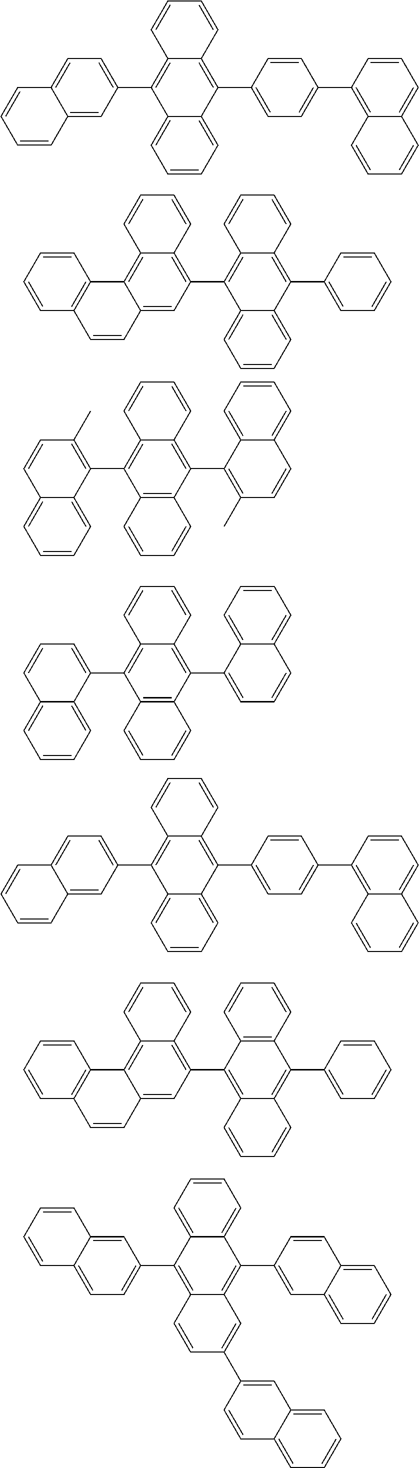

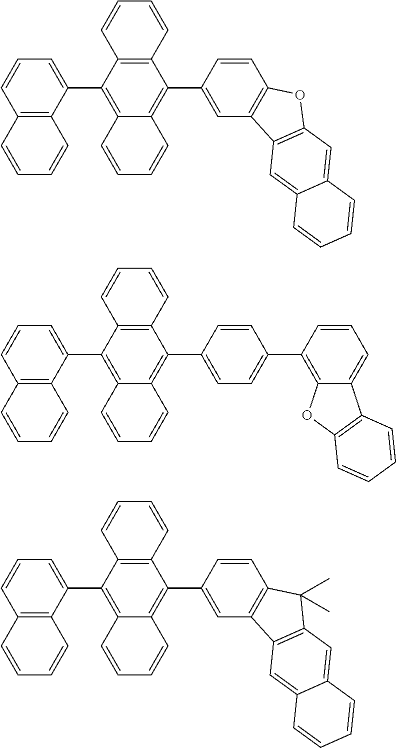

In some embodiments, the anthracene-based singlet host material is deuterated, i.e. the host material molecule contains at least one deuterium atom, such examples are disclosed in the patent literature such as CN102369256B, CN102428158B, CN102639671B, US2015021586A1, etc. The specific examples include:

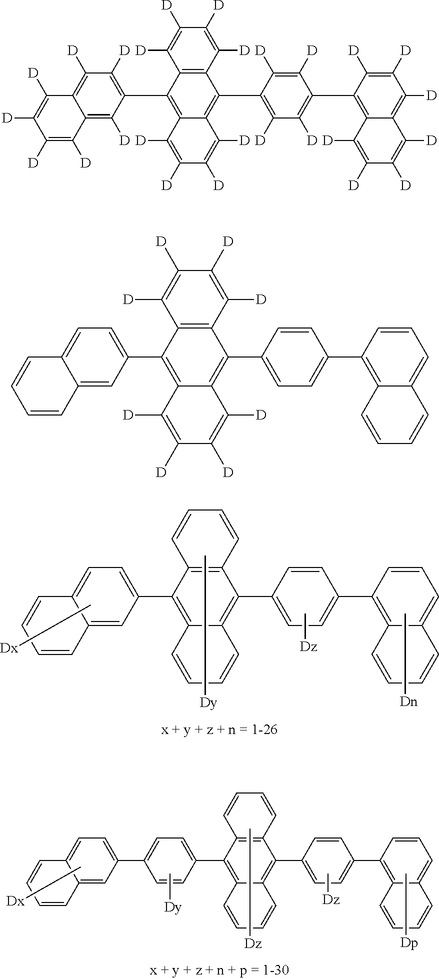

### Fluorescent Emitting Material

The singlet emitter tends to have a long conjugated π-electron system. Hitherto, there have been many examples of styryl amines and derivatives thereof as disclosed in JP2913116B and WO2001021729A1, and indenofluorenes and derivatives thereof as disclosed in WO2008006449 and WO2007140847.

In some embodiments, the singlet emitter can be selected from the group consisting of monostyrylamines, distyrylamines, tristyrylamines, tetrastyrylamines, styrenphosphines, styrenethers, and arylamines.

A monostyrylamine refers to a compound which comprises one unsubstituted or substituted styryl group and at least one amine, most preferably an aryl amine. Distyrylamine refers to a compound comprising two unsubstituted or substituted styryl groups and at least one amine, most preferably an aryl amine. Ternarystyrylamine refers to a compound which comprises three unsubstituted or substituted styryl groups and at least one amine, most preferably an aryl amine. Quaternarystyrylamine refers to a compound comprising four unsubstituted or substituted styryl groups and at least one amine, most preferably an aryl amine. Preferred styrene is stilbene, which may be further substituted. The corresponding phosphines and ethers are defined similarly as amines. Aryl amine or aromatic amine refers to a compound comprising three unsubstituted or substituted cyclic or heterocyclic aryl systems directly attached to nitrogen. At least one of these cyclic or heterocyclic aryl systems is preferably selected from fused ring systems and most preferably has at least 14 aryl ring atoms. Among the preferred examples are aryl anthramine, aryl anthradiamine, aryl pyrene amines, aryl pyrene diamines, aryl chrysene amines and aryl chrysene diamine. Aryl anthramine refers to a compound in which one diarylamino group is directly attached to anthracene, most preferably at position 9. Aryl anthradiamine refers to a compound in which two diarylamino groups are directly attached to anthracene, most preferably at positions 9,10. Aryl pyrene amines, aryl pyrene diamines, aryl chrysene amines and aryl chrysene diamine are similarly defined, where the diarylarylamino group is most preferably attached to position 1 or 1,6 of pyrene.

Examples of singlet emitters based on vinylamines and arylamines, which are also preferred, may be found in the following patent documents: WO2006000388, WO2006058737, WO2006000389, WO2007065549, WO2007115610, U.S. Pat. No. 7,250,532B2, DE102005058557A1, CN1583691A, JP08053397A, U.S. Pat. No. 6,251,531B1, US2006210830A, EP1957606A1, and US20080113101A1. The patent documents listed above are specially incorporated herein by reference in their entirety.

Examples of singlet emitters based on stilbene and its derivatives may be found in U.S. Pat. No. 5,121,029.

Further preferred singlet emitter can be selected from the group consisting of indenofluorene-amine and indenofluorene-diamine, as disclosed in WO2006122630, benzoindenofluorene-amine and benzoindenofluorene-diamine, as disclosed in WO2008006449, dibenzoindenofluorene-amine and dibenzoindenofluorene-diamine, as disclosed in WO2007140847.

Other materials that can be used as singlet emitter include polycyclic aromatic hydrocarbon compounds, in particular selected from the derivatives of the following compounds: anthracene such as 9,10-di(2-naphthyl)anthracene, naphthalene, tetraphenyl, phenanthrene, perylene such as 2,5,8,11-tetra-t-butylatedylene, indenoperylene, phenylene (benzo fused ring such as 4,4′-(bis(9-ethyl-3-carbazovinylene)-1,1′-biphenyl)), periflanthene, decacyclene, coronene, fluorene, spirobifluorene, arylpyren (e.g., US20060222886), arylenevinylene (e.g. U.S. Pat. Nos. 5,121,029, 5,130,603), cyclopentadiene such as tetraphenylcyclopentadiene, rubrene, coumarine, rhodamine, quinacridone, pyrane such as 4 (dicyanoethylene)-6-(4-dimethylaminostyryl-2-methyl)-4H-pyrane (DCM), thiapyran, bis (azinyl) imine-boron compounds (e.g. US20070092753A1), bis (azinyl) methene compounds, carbostyryl compounds, oxazone, benzoxazole, benzothiazole, benzimidazole, or diketopyrrolopyrrole. Some singlet emitter materials may be found in the following patent documents: US20070252517A1, U.S. Pat. Nos. 4,769,292, 6,020,078. The patent documents listed above are specially incorporated herein by reference in their entirety.

The following are some examples of singlet emitters:

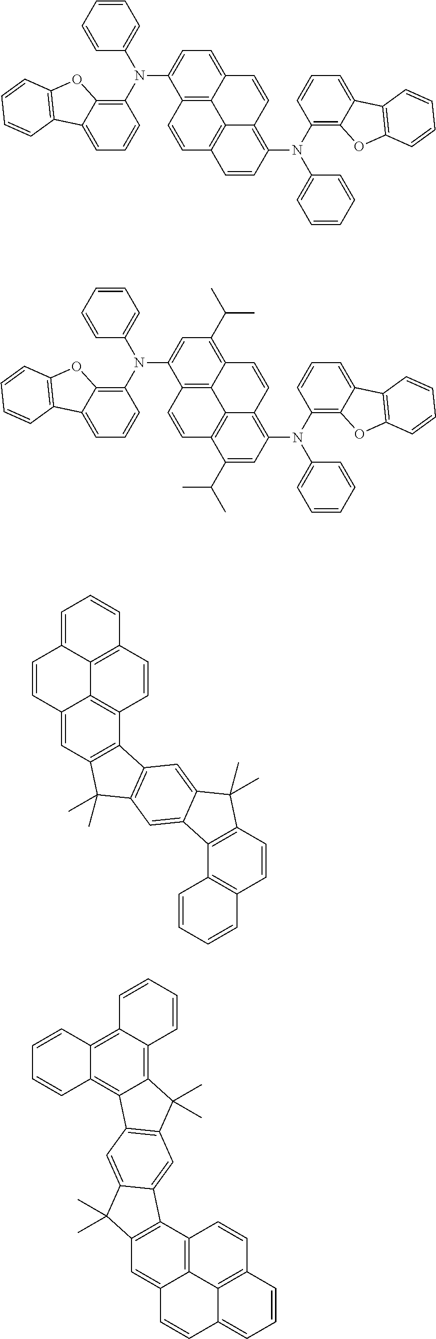

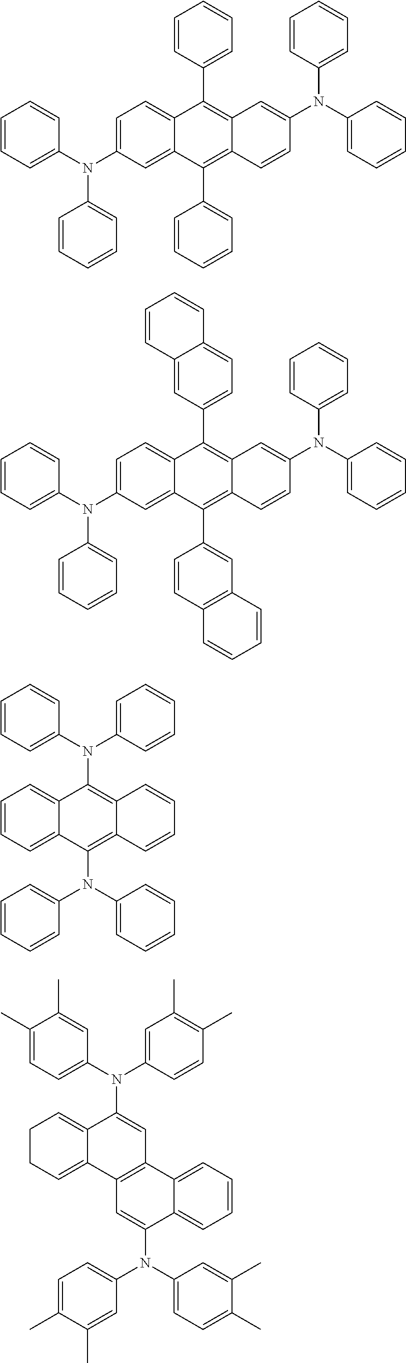

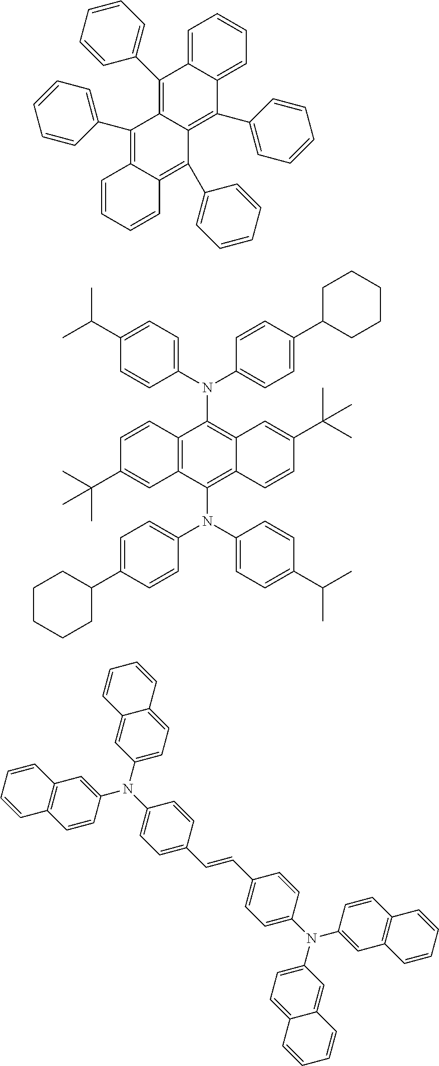

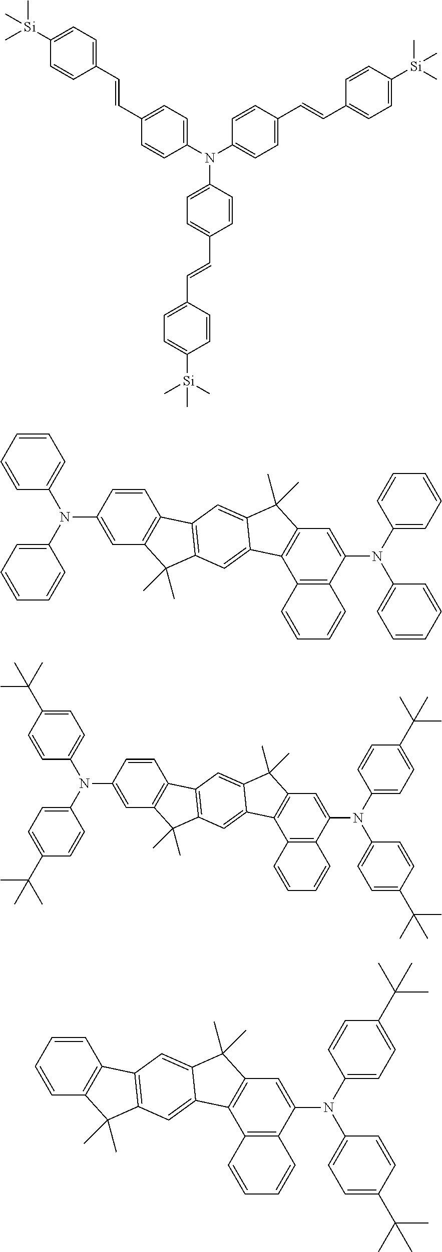

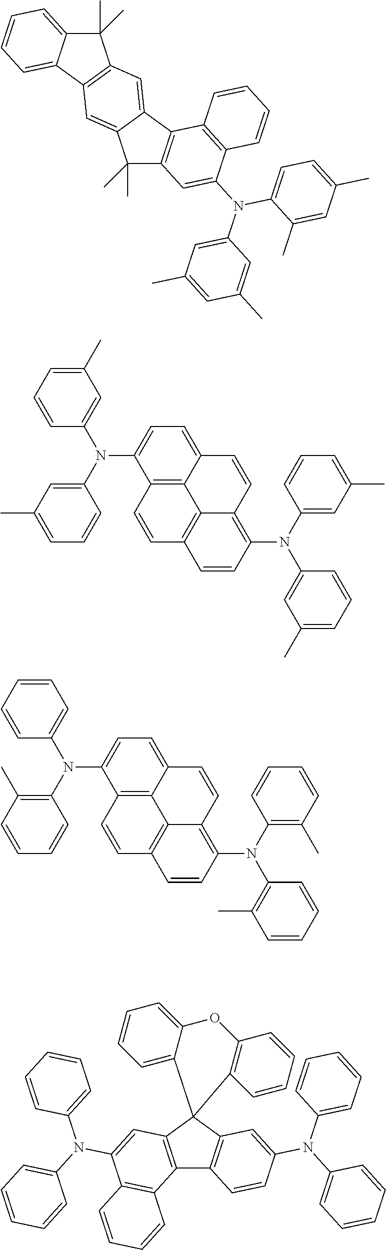

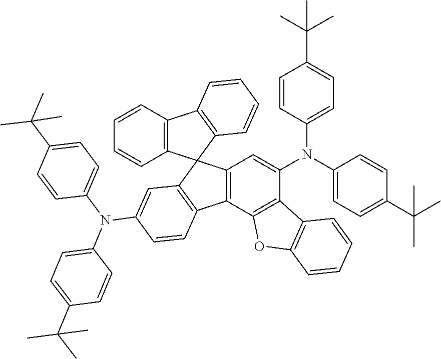 [SMILES: CC(C)(C)c1ccc(N(c2ccc(C(C)(C)C)cc2)c2ccc3c(c2)C2(c4ccccc4-c4ccccc42)c2cc(N(c4ccc(C(C)(C)C)cc4)c4ccc(C(C)(C)C)cc4)c4c(oc5ccccc54)c2-3)cc1]

It is an object of the present disclosure to provide a material for the evaporation-based OLEDs.

In some embodiments, the molecular weight of the organic compound≤1100 g/mol, preferably ≤1000 g/mol, more preferably ≤950 g/mol, furthermore preferably ≤900 g/mol, and most preferably ≤800 g/mol.

Another object of the present disclosure is to provide a material for the printed OLEDs.

In some embodiments, the molecular weight of the organic compound≥700 g/mol, preferably ≥800 g/mol, more preferably ≥900 g/mol, furthermore preferably ≥1000 g/mol, and most preferably ≥1100 g/mol.

In some embodiments, the organic compound as described herein has a solubility of ≥10 mg/mL in toluene at 25° C., preferably ≥15 mg/mL, and most preferably ≥20 mg/mL.

In yet another aspect, the present disclosure further provides to a formulation or ink comprising an organic compound, or a polymer as described above, and at least one organic solvent.

The viscosity and surface tension of the ink are important parameters in printing processes. A suitable ink surface tension is required for the specific substrates and the specific printing methods.

In some embodiments, the surface tension of the ink as described herein at 25° C. is in the range of 19 dyne/cm to 50 dyne/cm; more preferably in the range of 22 dyne/cm to 35 dyne/cm; and most preferably in the range of 25 dyne/cm to 33 dyne/cm.

In some embodiments, the viscosity of the ink as described herein at 25° C. is in the range of from about 1 cps to 100 cps; particularly in the range of 1 cps to 50 cps; more particularly in the range of 1.5 cps to 20 cps; and most particularly in the range of 4 cps to 20 cps. The resulting formulation will be particularly suitable for ink-jet printing.

The viscosity can be adjusted by different methods, such as by the selection of appropriate solvent and the concentration of the functional materials in the ink. In the ink comprising the above-mentioned metal-organic complexes or polymers as described herein facilitate the adjustment of the printing ink in the appropriate range according to the printing method used. Generally, in the formulation comprising the functional material as described herein, the weight ratio of the functional material ranges from 0.3 wt % to 30 wt %, preferably in the range of 0.5 wt % to 20 wt %, more preferably in the range of 0.5 wt % to 15 wt %, further preferably in the range of 0.5 wt % to 10 wt %, and most preferably in the range of 1 wt % to 5 wt %.

In some embodiments, the at least one organic solvent of the ink as described herein is selected from aromatic-based or heteroaromatic-based solvents, particular in aliphatic chain/ring substituted aromatic solvents, aromatic ketone solvents, or aromatic ether solvents.

Examples of solvents suitable for the present disclosure include, but not limited to aromatic-based or heteroaromatic-based solvents, such as p-diisopropylbenzene, amylbenzene, tetralin, cyclohexylbenzene, chloronaphtalene, 1,4-dimethylnaphthalene, 3-isopropylbenzene, p-methylisopropylbenzene, dipentylbenzene, tripentylbenzene, pentyltoluene, o-xylene, m-xylene, p-xylene, o-diethylbenzene, m-diethylbenzene, p-diethylbenzene, 1,2,3,4-tetramethylbenzene, 1,2,3,5-tetramethylbenzene, 1,2,4,5-tetramethylbenzene, butylbenzene, dodecylbenzene, dihexylbenzene, dibutylbenzene, p-diiisopropylbenzene, 1-methoxynaphthalene, cyclohexylbenzene, dimethylnaphthalene, 3-isopropylbiphenyl, p-methylisopropylbenzene, 1-methylnaphthalene, 1,2,4-trichlorobenzene, 1,3-dipropoxybenzene, 4,4-difluorobenzenemethane, 1,2-dimethoxy-4-(1-propenyl)benzene, diphenylmethane, 2-phenylpyridine, 3-phenylpyridine, N-methyldiphenylamine, 4-isopropylbipheny, α,α-dichlorodiphenylmethane, 4-(3-phenylpropyl)pyridine, benzylbenzoatel, 1,1-bis(3,4-dimethylphenyl) ethane, 2-isopropylnaphthalene, dibenzyl ether, etc; ketone-based solvents, such as 1-tetrahydronaphthalone, 2-tetrahydronaphthalone, 2-(phenylepoxy)tetrahydronaphthalone, 6-(methoxy)tetrahydronaphthalone, acetophenone, phenylacetone, benzophenone, and derivatives thereof such as 4-methyl acetophenone, 3-methyl acetophenone, 2-methyl acetophenone, 4-methyl propanone, 3-methyl propanone, 2-methyl propanone, isophorone, 2,6,8-trimethyl-4-nonanone, fenchone, 2-nonanone, 3-nonanone, 5-nonanone, 2-decanone, 2,5-hexanedione, phoron, di-n-amylketone, etc; aromatic ether solvents, such as 3-phenoxytoluene, butoxybenzene, benzylbutylbenzene, p-anisaldehyde dimethyl acetal, tetrahydro-2-phenoxy-2H-pyran, 1,2-dimethoxy-4-(1-propenyl)benzene, 1,4-benzodioxane, 1,3-dipropylbenzene, 2,5-dimethoxytoluene, 4-ethylphenyl ether, 1,2,4-trimethoxybenzene, 4-(1-propenyl)-1,2-dimethoxybenzene, 1,3-dimethoxybenzene, glycidyl phenyl ether, dibenzyl ether, 4-tert-butyl anisole, trans-anethole, 1,2-dimethoxybenzene, 1-methoxynaphthalene, diphenyl ether, 2-phenoxymethyl ether, 2-phenoxytetrahydrofuran, ethyl-2-naphthyl ether, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc; ester solvent, such as alkyl octanoate, alkyl sebacate, alkyl stearate, alkyl benzoate, alkyl phenylacetate, alkyl cinnamate, alkyl oxalate, alkyl maleate, alkyl lactone, alkyl oleate, etc.

Further, the at least one organic solvent of the ink as described herein can be selected from aliphatic ketones, such as 2-nonanone, 3-nonanone, 5-nonanone, 2-decanone, 2,5-hexanedione, 2,6,8-trimethyl-4-nonanone, phorone, 6-undecanone, etc; and the at least one organic solvent of the ink can be selected from aliphatic, ethers, such as, dipentyl ether, hexyl ether, dioctyl ether, ethylene glycol dibutyl ether, diethylene glycol diethyl ether, diethylene glycol butyl methyl ether, diethylene glycol dibutyl ether, triethylene glycol dimethyl ether, triethylene glycol ethyl methyl ether, triethylene glycol butyl methyl ether, tripropylene glycol dimethyl ether, tetraethylene glycol dimethyl ether, etc.

In some embodiments, the printing ink further comprises another organic solvent. Examples of the another organic solvent include, but not limited to: methanol, ethanol, 2-methoxyethanol, dichloromethane, trichloromethane, chlorobenzene, o-dichlorobenzene, tetrahydrofuran, anisole, morpholine, toluene, o-xylene, m-xylene, p-xylene, 1,4-dioxane, acetone, methyl ethyl ketone, 1,2-dichloroethane, 3-phenoxytoluene, 1,1,1-trichloroethane, 1,1,2,2-tetrachloroethane, ethyl acetate, butyl acetate, dimethylformamide, dimethylacetamide, dimethylsulfoxide, tetraline, decalin, indene, and/or mixtures thereof.

In some embodiments, the formulation as described herein is a solution.

In some embodiments, the formulation as described herein is a dispersion.

The formulations in the embodiments as described herein may comprise the organic compound or mixture thereof of 0.01 wt % to 20 wt %, preferably 0.1 wt % to 15 wt %, more preferably 0.2 wt % to 10 wt %, and most preferably 0.25 wt % to 5 wt %.

The present disclosure further provides the use of the formulation as a coating or printing ink in the preparation of organic electronic devices, particularly preferably by printing or coating processing methods.

Where suitable printing or coating techniques include, but not limited to ink-jet printing, nozzle printing, gravure printing, typographic printing, screen printing, dip coating, spin coating, blade coating, roller printing, torsion roll printing, planographic printing, flexographic printing, rotary printing, spray coating, brush coating, pad printing, slit die coating, and so on. Preferred techniques are ink-jet printing, nozzle printing, and gravure printing. The solution or dispersion may additionally comprise one or more components, such as surface active compounds, lubricants, wetting agents, dispersing agents, hydrophobic agents, binders, etc., which are used to adjust the viscosity and film forming properties, or to improve adhesion, etc. For more information on printing technologies and their requirements for solutions, such as solvent, concentration, viscosity, etc, please refer to Handbook of Print Media: Technologies and Production Methods, edited by Helmut Kipphan, ISBN 3-540-67326-1.

Based on the organic compound, the present disclosure also provides an application of the organic compound or polymer as described above, i.e., the organic compound or polymer is applied to an organic electronic device, and the organic electronic device may be selected from, but not limited to an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic electronic device, an organic sensor, an organic plasmon emitting diode (OPED), etc., particularly preferably is an organic electroluminescent device, such as an OLED, an OLEEC, an organic light emitting field effect transistor. In the embodiments of the present disclosure, it is preferred to use the organic compound for the light-emitting layer of the organic electroluminescent device.

In yet another aspect, the present disclosure further provides an organic electronic device comprising an organic compound, or a polymer, or a mixture as described above. Generally, such organic electronic device comprises a cathode, an anode, and a functional layer disposed between the cathode and the anode, where the functional layer comprises an organic compound, or a polymer, or a mixture as described above. The organic electronic device may be selected from, but not limited to, an organic light-emitting diode (OLED), an organic photovoltaic cell (OPV), an organic light emitting electrochemical cell (OLEEC), an organic field effect transistor (OFET), an organic light emitting field effect transistor, an organic laser, an organic spintronic device, an organic sensor, or an organic plasmon emitting diode (OPED), etc., particularly preferably is an organic electroluminescent device, such as an OLED, an OLEEC, an organic light emitting field effect transistor.

In some embodiments, the electroluminescent device comprises a light-emitting layer, the light-emitting layer comprises an organic compound, or a polymer, or a mixture as described above; or comprising an organic compound or a polymer as described above, and a phosphorescent emitter; or comprising an organic compound or a polymer as described above, and a host material; or comprising an organic compound or a polymer as described above, a phosphorescent emitter, and a host material.

In the electroluminescent devices as described above, in particular an OLED, which comprises a substrate, an anode, at least one light-emitting layer, and a cathode.

The substrate should be opaque or transparent. A transparent substrate could be used to produce a transparent light-emitting device (for example: Bulovic et al., Nature 1996, 380, p29, and Gu et al., Appl. Phys. Lett. 1996, 68, p2606). The substrate can be rigid/flexible, e.g. it can be plastic, metal, semiconductor wafer, or glass. Preferably, the substrate has a smooth surface. Particularly desirable are substrates without surface defects. In some embodiments, the substrate is flexible and can be selected from a polymer film or plastic with a glass transition temperature (Tg)>150° C., preferably >200° C., more preferably >250° C., and most preferably >300° C. Examples of the suitable flexible substrate includes poly ethylene terephthalate (PET) and polyethylene glycol (2,6-naphthalene) (PEN).

The choice of anodes may include a conductive metal, or a metal oxide, or a conductive polymer. The anode should be able to easily inject holes into a hole-injection layer (HIL), a hole-transport layer (HTL), or a light-emitting layer. In some embodiments, the absolute value of the difference between the work function of the anode and the HOMO energy level of the emitter of the light-emitting layer, or the HOMO energy level/valence band energy level of the p-type semiconductor materials of the hole-injection layer (HIL)/hole-transport layer (HTL)/electron-blocking layer (EBL)<0.5 eV, preferably <0.3 eV, and most preferably <0.2 eV. Examples of anode materials may include, but not limited to: Al, Cu, Au, Ag, Mg, Fe, Co, Ni, Mn, Pd, Pt, ITO, aluminum-doped zinc oxide (AZO), etc. Other suitable anode materials are known and can be readily selected for use by the general technicians in this field. The anode material can be deposited using any suitable technique, such as a suitable physical vapor deposition method, including RF magnetron sputtering, vacuum thermal evaporation, e-beam, etc. In some embodiments, the anode is patterned. Patterned conductive ITO substrates are commercially available and can be used to produce the devices as described herein.

The choice of cathode may include a conductive metal or a metal oxide. The cathode should be able to easily inject electrons into the electron-injection layer (EIL), the electron-transport layer (ETL), or the directly into the light-emitting layer. In some embodiments, the absolute value of the difference between the work function of the cathode and the LUMO energy level of the emitter of the light-emitting layer, or the LUMO energy level/conduction band energy level of the n-type semiconductor materials of the electron-injection layer (EIL)/electron-transport layer (ETL)/hole-blocking layer (HBL)<0.5 eV, preferably <0.3 eV, and most preferably <0.2 eV. In principle, all materials that can be used as cathodes for OLEDs may be applied as cathode materials for the devices as described herein. Examples of cathode materials include, but not limited to: Al, Au, Ag, Ca, Ba, Mg, LiF/Al, MgAg alloy, BaF₂/Al, Cu, Fe, Co, Ni, Mn, Pd, Pt, ITO, etc. The cathode material can be deposited using any suitable technique, such as the suitable physical vapor deposition method, including RF magnetron sputtering, vacuum thermal evaporation, e-beam, etc.

The OLED may also comprise other functional layers, such as a hole-injection layer (HIL), a hole-transport layer (HTL), an electron-blocking layer (EBL), an electron-injection layer (EIL), an electron-transport layer (ETL), and a hole-blocking layer (HBL). Materials suitable for use in these functional layers are described in details above and in WO2010135519A1, US20090134784A1 and WO2011110277A1, the entire contents of these three documents are hereby incorporated herein for reference.

In some embodiments, the light-emitting layer of the light-emitting device is prepared by vacuum evaporation deposition of the organic compound as described herein.

In some embodiments, the light-emitting layer of the light-emitting device is prepared by the formulation as described herein.

The light-emitting device as described herein has a light-emitting wavelength between 300 nm and 1000 nm, preferably between 350 nm and 900 nm, more preferably between 400 nm and 800 nm.

The present disclosure further provides the applications of organic electronic devices in various electronic equipment, including, but not limited to, display devices, lighting equipments, light sources, sensors, etc.

The present disclosure further provides electronic devices comprising organic electronic devices as described herein, including, but not limited to, display devices, lighting equipments, light sources, sensors, etc.

The present disclosure will be described below in conjunction with the preferred embodiments, but the present disclosure is not limited to the following embodiments. It should be understood that the scope of the present disclosure is covered by the scope of the claims of the present disclosure, and those skilled in the art should understand that certain changes may be made to the embodiments of the present disclosure.

### Specific Embodiment

### Example 1

The synthetic route of the compound 1 is shown below:

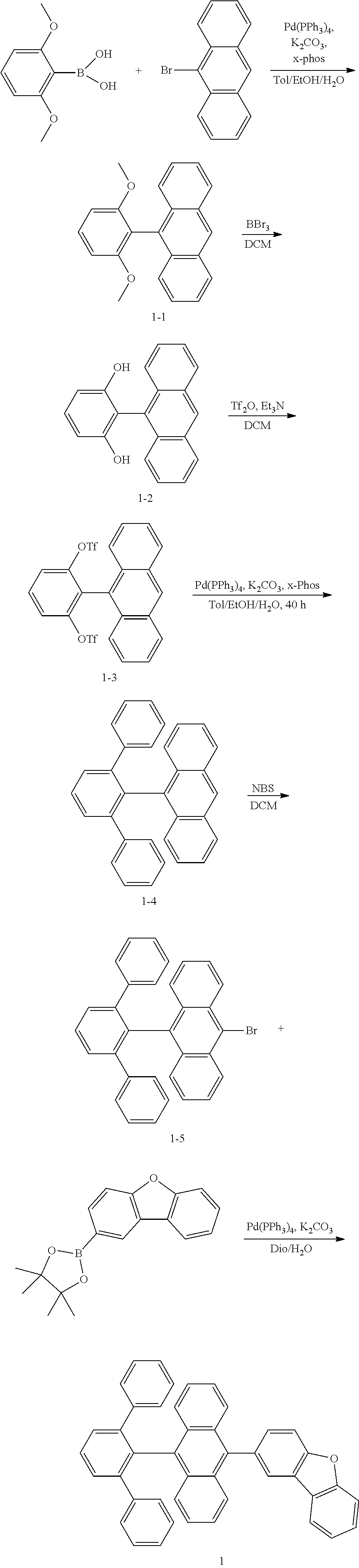

9-Bromoanthracene (51.4 g, 0.2 mol), 2,6-dimethoxyphenylboronic acid (72.8 g, 0.4 mol), potassium carbonate (55.2 g, 0.4 mol), x-phos (9.56 g, 40 mmol), and tetrakis(triphenylphosphine)palladium (6.9 g, 12 mmol) were added in turn to a 1 L three-necked flask. After adding 400 mL of toluene, 100 mL of ethanol, and 100 mL of water, the resulting mixture was heated to 90° C. under N₂ atmosphere and reacted for overnight. After cooling down to room temperature, 500 mL of water was added, then the resulting mixture separated, and the aqueous phase was extracted with DCM (300 mL*3). The organic phases were then combined, dried over anhydrous Na₂SO₄, the excess solvent was removed under reduced pressure distillation, then the resulting sample was further purified by silica gel column chromatography (eluent: petroleum ether) to yield about 29.8 g (about 47% yield) of compound 1-1. MS (ASAP)=314.4.

Compound 1-1 (29.5 g, 0.09 mol) was added into a 500 mL three-necked flask, then about 300 mL of anhydrous dichloromethane was added. After cooling down to −78° C. in a liquid nitrogen ethanol bath, boron tribromide (35 mL, 0.36 mol) was added dropwise into the system, then the resulting mixture was heated to room temperature and reacted for 3 h. After adding 30 mL of methanol dropwise to the reaction system under N₂ atmosphere, the excess boron tribromide was quenched, then saturated aqueous sodium carbonate solution was added to the reaction system until the solution was neutral. After the separation, the aqueous phase was extracted with DCM (300 mL*3), the organic phases were combined and dried over anhydrous Na₂SO₄, the excess solvent was removed under reduced pressure distillation, then the resulting sample was purified by silica gel column chromatography (eluent: DCM) to yield about 25 g (about 93% yield) of compound 1-2. MS (ASAP)=286.3.

Compound 1-2 (25 g, 88 mmol) was added into a 500 mL three-necked flask, then about 300 mL of anhydrous dichloromethane was added. After adding triethylamine (35.6 g, 352 mmol) under N₂ atmosphere, the result was cooled down to about 0° C. in an ice-salt bath, trifluoromethanesulfonic anhydride (99.3 g, 352 mmol) was added dropwise into the above mixture, then the resulting mixture was naturally heated to room temperature and reacted for overnight. After the reaction was completed, the reaction solution was removed to obtain crude product, then 100 mL of methanol was added. After the sonication, the result was filtrated and the residue was further washed with petroleum ether (80 mL*3) to obtained about 39 g (about 80% yield) of compound 1-3. MS (ASAP)=550.4.

Compound 1-3 (39 g, 71 mmol), phenylboronic acid (34.6 g, 284 mmol), potassium carbonate (39.2 g, 284 mmol), x-phos (3.4 g, 7.1 mmol), and tetrakis(triphenylphosphine)palladium (2.3 g, 2.1 mmol) were added in turn to a 1 L three-necked flask. After adding 500 mL of 1,4-dioxane and 100 mL of water, the resulting mixture was heated to 90° C. under N₂ atmosphere and reacted for overnight. After cooling down to room temperature, the result was diluted with 500 mL of water, then extracted with DCM (300 mL*3). The organic phases were then combined, dried over anhydrous Na₂SO₄, the excess solvent was removed under reduced pressure distillation, then the resulting sample was further purified by silica gel column chromatography (eluent: petroleum ether) to yield about 20.7 g (about 72% yield) of compound 1-4. MS (ASAP)=406.5.

Compound 1-4 (20.5 g, 50 mmol) was added into a 500 mL three-necked flask, then about 100 mL of anhydrous dichloromethane was added. After heating up to 60° C. under N₂ atmosphere, dichloromethane solution of NB S (9.8 g, 55 mmol) was added dropwise into the reaction system, then the reaction was reacted for 2 h in dark. After cooling down to room temperature, the excess solvent was removed under reduced pressure distillation, then the result was ultrasonically washed with 100 mL of methanol and filtrated to obtain 22.3 g (about 92% yield) of compound 1-5. MS (ASAP)=485.4.

Compound 1-5 (22 g, 45 mmol), dibenzofuran-2-boronic acid pinacol ester (14.5 g, 49.5 mmol), tetrakis(triphenylphosphine)palladium (2.5 g, 2.3 mmol), and potassium carbonate (12.4 g, 90 mmol) were added in turn to a 500 mL three-necked flask. After adding 240 mL of 1,4-dioxane and 40 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 20 g (about 78% yield) of compound 1. MS (ASAP)=572.7.

### Example 2

The synthetic route of the compound 2 is shown below:

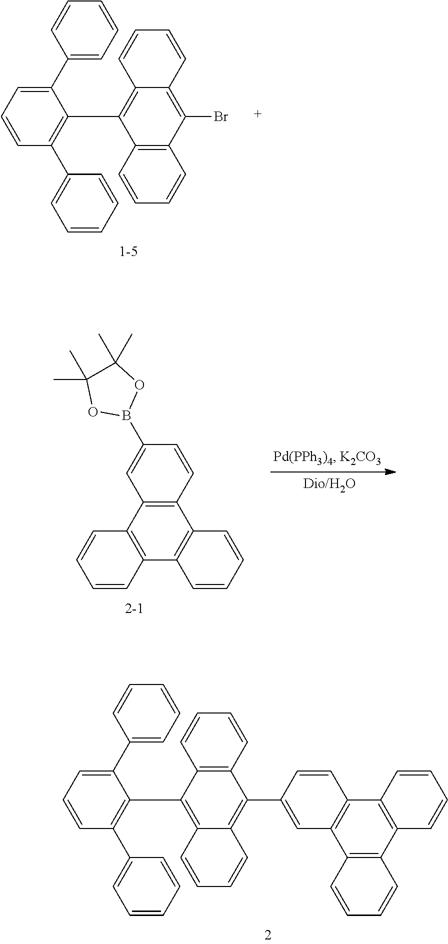

Compound 1-5 (12 g, 25 mmol), pinacol ester of tritylene-2-boronic acid (9.7 g, 27.5 mmol), tetrakis(triphenylphosphine)palladium (1.5 g, 1.3 mmol), and potassium carbonate (6.9 g, 50 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 12.6 g (about 80% yield) of compound 2. MS (ASAP)=632.8.

### Example 3

The synthetic route of the compound 3 is shown below:

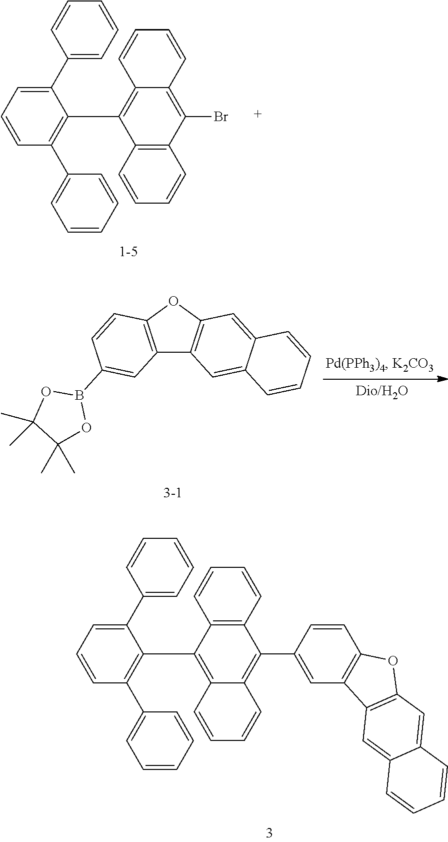 [SMILES: Brc1c2ccccc2c(-c2c(-c3ccccc3)cccc2-c2ccccc2)c2ccccc12 ; CC1(C)OB(c2ccc3oc4cc5ccccc5cc4c3c2)OC1(C)C ; c1ccc(-c2cccc(-c3ccccc3)c2-c2c3ccccc3c(-c3ccc4oc5cc6ccccc6cc5c4c3)c3ccccc23)cc1]

Compound 1-5 (13.1 g, 27 mmol), compound 3-1 (10.2 g, 29.7 mmol), tetrakis(triphenylphosphine)palladium (1.5 g, 1.3 mmol), and potassium carbonate (7.5 g, 54 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 13.5 g (about 80% yield) of compound 3. MS (ASAP)=622.8.

### Example 4

The synthetic route of the compound 4 is shown below:

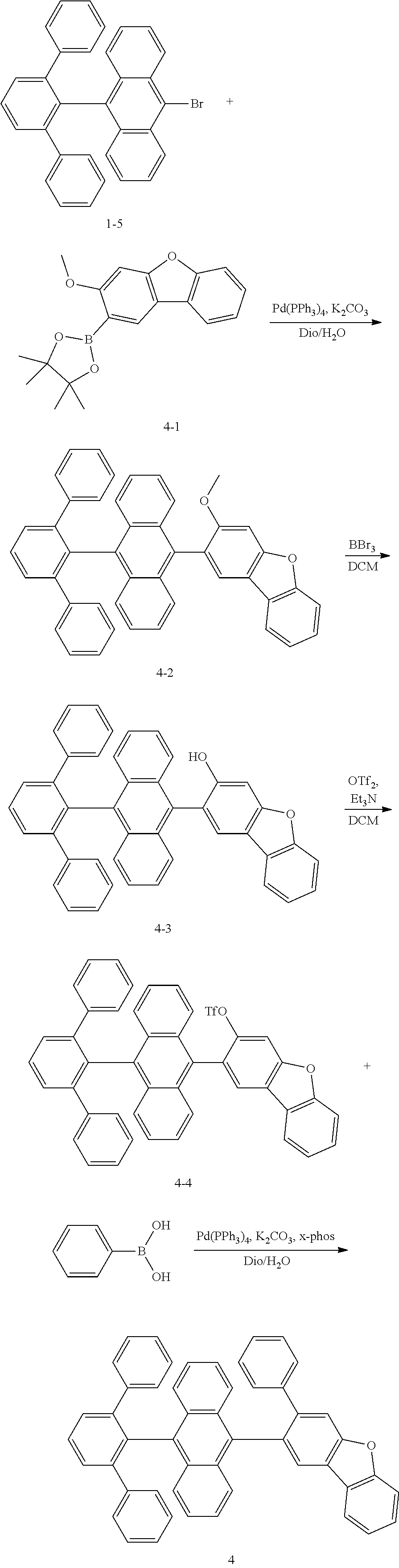

Compound 1-5 (10 g, 21 mmol), compound 4-1 (7.5 g, 23 mmol), tetrakis(triphenylphosphine)palladium (1.1 g, 1 mmol), and potassium carbonate (5.8 g, 42 mmol) were added in turn to a 250 mL three-necked flask. After adding 100 mL of 1,4-dioxane and 20 mL of water, the resulting mixture was heated to 90° C. under N₂ atmosphere and reacted for overnight. After cooling down to room temperature, 100 mL of water was added, then the result was extracted with DCM (300 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 8.2 g (about 65% yield) of compound 4-2. MS (ASAP)=602.7.

Compound 4-2 (8 g, 13 mmol) was added into a 250 mL three-necked flask, then about 100 mL of anhydrous dichloromethane was added. After cooling down to −78° C. in a liquid nitrogen ethanol bath, boron tribromide (2.5 mL, 26 mmol) was added dropwise into the system, then the resulting mixture was heated to room temperature and reacted for 3 h. After adding 10 mL of methanol dropwise to the reaction system under N₂ atmosphere, the excess boron tribromide was quenched, then saturated aqueous sodium carbonate solution was added to the reaction system until the solution system is neutral. After the separation, the aqueous phase was extracted with DCM (300 mL*3), the organic phases were combined and dried over anhydrous Na₂SO₄, then the excess solvent was removed under reduced pressure distillation, and the resulting sample was purified by silica gel column chromatography (eluent: DCM) to yield 6.9 g (about 90% yield) of compound 4-3. MS (ASAP)=588.7.

Compound 4-3 (6.9 g, 11.7 mmol) was added into a 250 mL three-necked flask, then about 300 mL of anhydrous dichloromethane was added. After adding triethylamine (2.4 g, 23.4 mmol) under N₂ atmosphere, the result was cooled down to about 0° C. in an ice-salt bath, trifluoromethanesulfonic anhydride (6.6 g, 23.4 mmol) was added dropwise into the above mixture, then the resulting mixture was naturally heated to room temperature and reacted for overnight. After the the reaction was completed, the reaction solution was removed to obtain crude product, then 50 mL of methanol was added. After the sonication, the result was filtrated and the residue was further washed with petroleum ether (80 mL*3) to obtained about 6.9 g (about 82% yield) of compound 4-4. MS (ASAP)=720.8.

Compound 4-4 (6.9 g, 9.5 mmol), phenylboronic acid (2.3 g, 19 mmol), potassium carbonate (2.6 g, 19 mmol), x-phos (0.45 g, 0.95 mmol), and tetrakis(triphenylphosphine)palladium (0.5 g, 0.48 mmol) were added in turn to a 250 mL three-necked flask. After adding 80 mL of 1,4-dioxane and 20 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 4.6 g (about 75% yield) of compound 4. MS (ASAP)=648.8.

### Example 5

The synthetic route of the compound 5 is shown below:

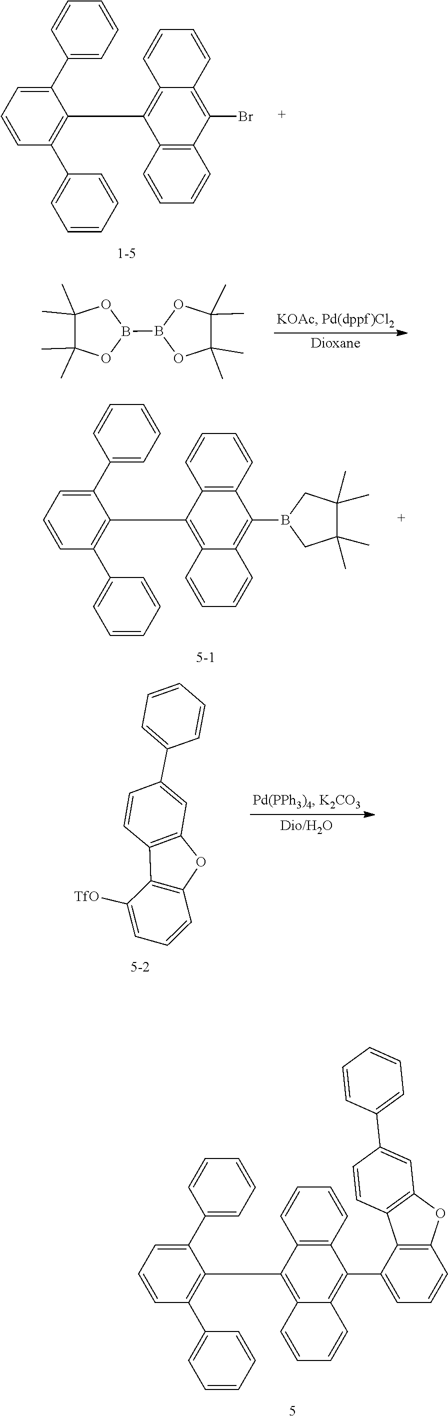

Compound 1-5 (10 g, 21 mmol), bis(pinacolato)diboron (8.0 g, 31.5 mmol), potassium acetate (3.1 g, 31.5 mmol), Pd(dppf)Cl₂ (0.7 g, 1 mmol) were added in turn to a 250 mL three-necked flask. After adding about 100 mL of anhydrous 1,4-dioxane, the resulting mixture was heated to 110° C. under N₂ atmosphere and reacted for 4 h. After the reaction was completed, the excess solvent was removed under reduced pressure distillation, then the resulting sample was further purified by silica gel column chromatography (eluent: PE:ECM=5:1) to yield 9.5 g (about 85% yield) of compound 5-1. MS (ASAP)=532.5.

Compound 5-1 (9.5 g, 17.8 mmol), compound 5-2 (7.0 g, 17.8 mmol), potassium carbonate (4.9 g, 35.6 mmol), and tetrakis(triphenylphosphine)palladium (1.0 g, 0.89 mmol) were added in turn to a 250 mL three-necked flask. After adding 100 mL of 1,4-dioxane and 20 mL of water, the resulting mixture was heated to 90° C. under N₂ atmosphere and reacted for overnight. After cooling down to room temperature, the result was diluted with 100 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 9.6 g (about 83% yield) of compound 5. MS (ASAP)=648.8.

### Example 6

The synthetic route of the compound 6 is shown below:

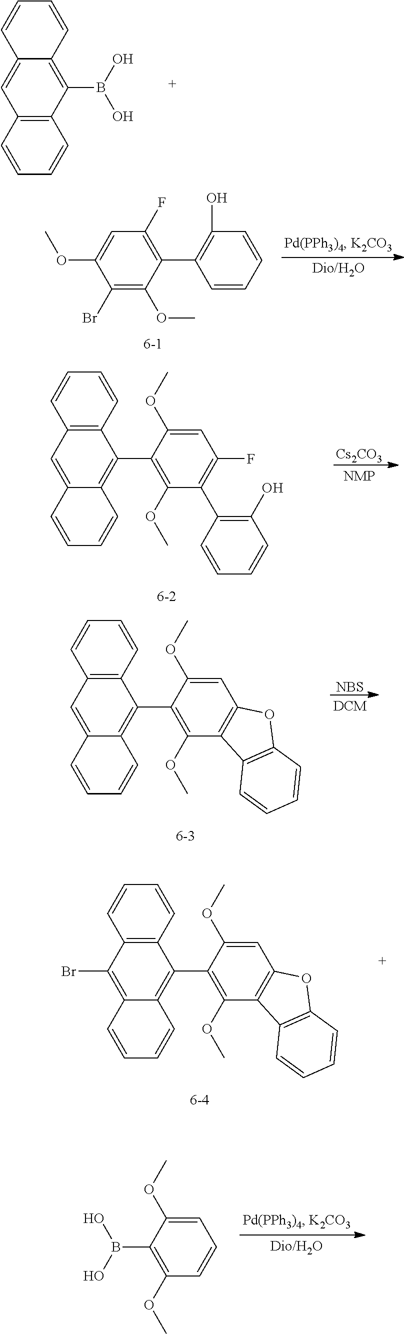

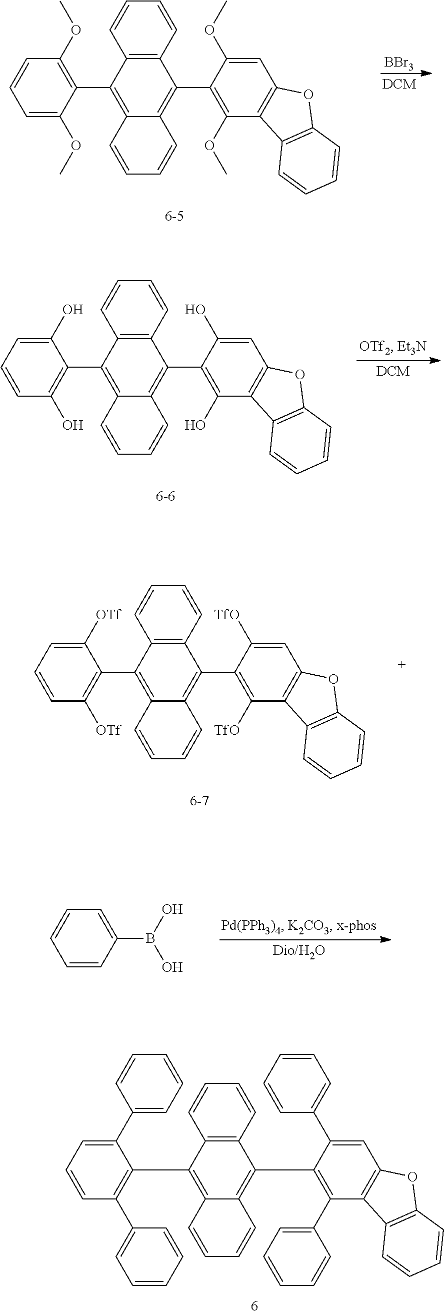 [SMILES: COc1cccc(OC)c1-c1c2ccccc2c(-c2c(OC)cc3oc4ccccc4c3c2OC)c2ccccc12 ; "BBr3" ; Oc1cccc(O)c1-c1c2ccccc2c(-c2c(O)cc3oc4ccccc4c3c2O)c2ccccc12 ; O=S(=O)(Oc1cccc(OS(=O)(=O)C(F)(F)F)c1-c1c2ccccc2c(-c2c(OS(=O)(=O)C(F)(F)F)cc3oc4ccccc4c3c2OS(=O)(=O)C(F)(F)F)c2ccccc12)C(F)(F)F ; OB(O)c1ccccc1 ; c1ccc(-c2cccc(-c3ccccc3)c2-c2c3ccccc3c(-c3c(-c4ccccc4)cc4oc5ccccc5c4c3-c3ccccc3)c3ccccc23)cc1]

9-Anthraceneboronic acid (88 g, 0.4 mol), compound 6-1 (129.6 g, 0.4 mol), potassium carbonate (82.8 g, 0.6 mol), tetrakis(triphenylphosphine)palladium (13.8 g, 12 mmol) were added in turn to a 2 L three-necked flask. After adding 1000 mL of 1,4-dioxane and 200 mL of water, the resulting mixture was heated to 90° C. under N₂ atmosphere and reacted for overnight. After cooling down to room temperature, 500 mL of water was added, then the resulting mixture was separated, and the aqueous phase was extracted with DCM (300 mL*3). The organic phases were then combined, dried over anhydrous Na₂SO₄, the excess solvent was removed under reduced pressure distillation, then the resulting sample was purified by silica gel column chromatography (eluent: PE:DCM=1:1) to yield about 144.3 g (about 85% yield) of compound 6-2. MS (ASAP)=424.5.

Compound 6-2 (144 g, 339 mmol) was added into a 2 L three-necked flask, then about 1000 mL of anhydrous N-methyl-2-pyrrolidone and cesium carbonate (165.8 g, 508 mmol) were added. After heating up to 140° C. under N₂ atmosphere, the resulting mixture was reacted for 4 h. After the the reaction was completed, the resulting mixture was filtrated, then the excess solvent was removed under reduced pressure distillation, and the resulting sample was purified by silica gel column chromatography (eluent: PE:DCM=5:1) to yield 116.6 g (about 85% yield) of compound 6-3. MS (ASAP)=404.5.

Compound 6-3 (116.5 g, 288 mmol) was added into a 2 L three-necked flask, the about 800 mL of anhydrous dichloromethane was added. After heating up to 60° C. under N₂ atmosphere, dichloromethane solution of NBS (56.4 g, 316.8 mmol) was added dropwise into the reaction system, then the reaction was reacted for 2 h in dark. After cooling down to room temperature, the excess solvent was removed under reduced pressure distillation, then the result was ultrasonically washed with 300 mL of methanol and filtrated to obtain 125.3 g (about 90% yield) of compound 6-4. MS (ASAP)=483.4.

Compound 6-4 (20 g, 41.3 mmol), 2,6-dimethoxyphenylboronic acid (15 g, 82.6 mmol), potassium carbonate (11.4 g, 82.6 mmol), and tetrakis(triphenylphosphine)palladium (2.3 g, 2.1 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, the result was diluted with 100 mL of water, then extracted with DCM (300 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 16.1 g (about 72% yield) of compound 6-5. MS (ASAP)=540.6.

Compound 6-5 (16 g, 29.6 mmol) was added into a 500 mL three-necked flask, then about 200 mL of anhydrous dichloromethane was added. After cooling down to −78° C. in a liquid nitrogen ethanol bath, boron tribromide (14 mL, 148 mmol) was added dropwise into the system, then the resulting mixture was heated to room temperature and reacted for 3 h. After adding 30 mL of methanol dropwise to the reaction system under N₂ atmosphere, the excess boron tribromide was quenched, then saturated aqueous sodium carbonate solution was added to the reaction system until the solution was neutral. After the separation, the aqueous phase was extracted with DCM (300 mL*3), the organic phases were combined and dried with anhydrous Na₂SO₄, the excess solvent was removed under reduced pressure distillation, then the resulting sample was purified by silica gel column chromatography (eluent: DCM) to yield about 12.9 g (about 90% yield) of compound 6-6. MS (ASAP)=484.5.

Compound 6-6 (12.9 g, 11.7 mmol) was added into a 250 mL three-necked flask, then about 300 mL of anhydrous dichloromethane was added. After adding triethylamine (7.1 g, 70.2 mmol) under N₂ atmosphere, the result was cooled down to about 0° C. in an ice-salt bath, trifluoromethanesulfonic anhydride (19.8 g, 70.2 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained about 8.9 g (about 75% yield) of compound 6-7. MS (ASAP)=1012.7.

Compound 6-7 (8.9 g, 8.8 mmol), phenylboronic acid (8.6 g, 70.4 mmol), potassium carbonate (9.7 g, 70.4 mmol), x-phos (0.42 g, 0.88 mmol), and tetrakis(triphenylphosphine)palladium (0.5 g, 0.44 mmol) were added in turn to a 250 mL three-necked flask. After adding 80 mL of 1,4-dioxane and 20 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 4.3 g (about 69% yield) of compound 6. MS (ASAP)=724.9.

### Example 7

The synthetic route of the compound 7 is shown below:

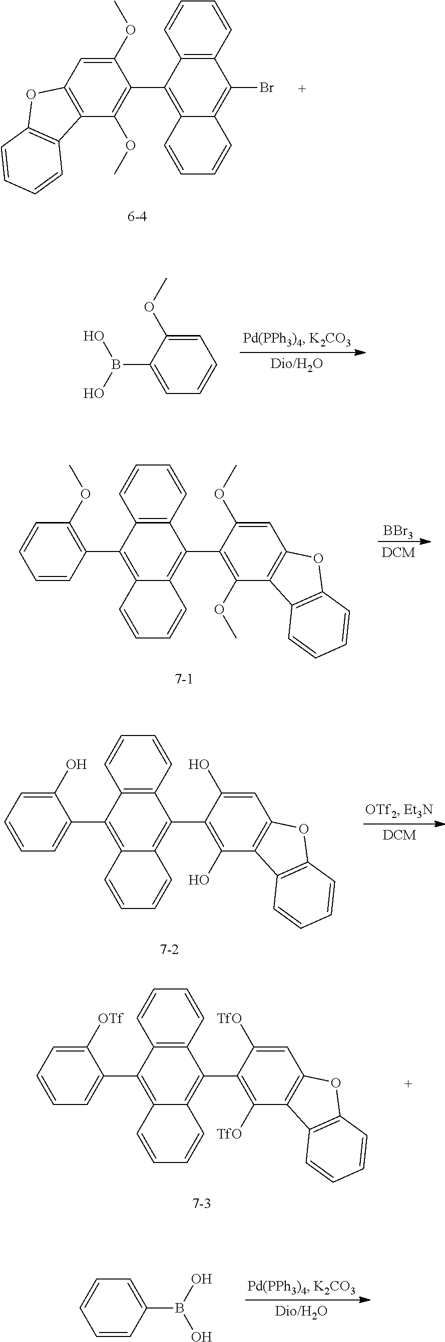

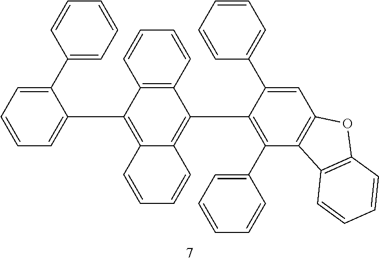 [SMILES: c1ccc(-c2ccccc2-c2c3ccccc3c(-c3c(-c4ccccc4)cc4oc5ccccc5c4c3-c3ccccc3)c3ccccc23)cc1]

Compound 6-4 (20 g, 41.3 mmol), 2-methoxyphenylboronic acid (12.6 g, 82.6 mmol), potassium carbonate (11.4 g, 82.6 mmol), and tetrakis(triphenylphosphine)palladium (2.3 g, 2.1 mmol) were added into a 500 mL three-necked flask. After adding 200 mL of 1,4-dioxane and 40 mL of water, the resulting mixture was heated to 90° C. under N₂ atmosphere and reacted for overnight. After cooling down to room temperature, the result was diluted with 100 mL of water, then extracted with DCM (30 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 16.9 g (about 80% yield) of compound 7-1. MS (ASAP)=510.6.

Compound 7-1 (16.9 g, 33 mmol) was added into a 500 mL three-necked flask, then about 200 mL of anhydrous dichloromethane was added. After cooling down to −78° C. in a liquid nitrogen ethanol bath, boron tribromide (12.5 mL, 132 mmol) was added dropwise into the system, then the resulting mixture was heated to room temperature and reacted for 3 h. After adding 30 mL of methanol dropwise to the reaction system under N₂ atmosphere, the excess boron tribromide was quenched, then saturated aqueous sodium carbonate solution was added to the reaction system until the solution system is neutral. After the separation, the aqueous phase was extracted with DCM (300 mL*3), the organic phases were combined and dried with anhydrous Na₂SO₄, the excess solvent was removed under reduced pressure distillation, then the resulting sample was purified by silica gel column chromatography (eluent: DCM) to yield 13.9 g (about 90% yield) of compound 7-2. MS (ASAP)=468.5.

Compound 7-2 (13.9 g, 29.6 mmol) was added into a 500 mL three-necked flask, then about 150 mL of anhydrous dichloromethane was added. After adding triethylamine (14.9 g, 148 mmol) under N₂ atmosphere, the result was cooled down to about 0° C. in an ice-salt bath, trifluoromethanesulfonic anhydride (41.7 g, 148 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained about 19.7 g (about 77% yield) of compound 7-3. MS (ASAP)=864.7.

Compound 7-3 (19.5 g, 22.6 mmol), phenylboronic acid (16.5 g, 135.6 mmol), potassium carbonate (18.7 g, 135.6 mmol), x-phos (1.1 g, 2.3 mmol), and tetrakis(triphenylphosphine)palladium (1.4 g, 1.2 mmol) were added in turn to a 500 mL three-necked flask. After adding about 200 mL of 1,4-dioxane and 40 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 9.5 g (about 65% yield) of compound 7. MS (ASAP)=648.8.

### Example 8

The synthetic route of the compound 8 is shown below:

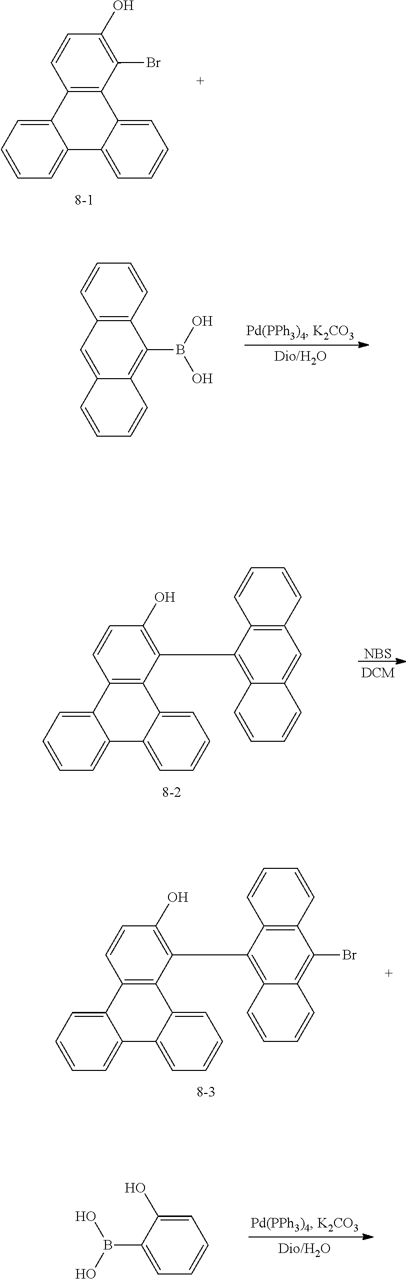

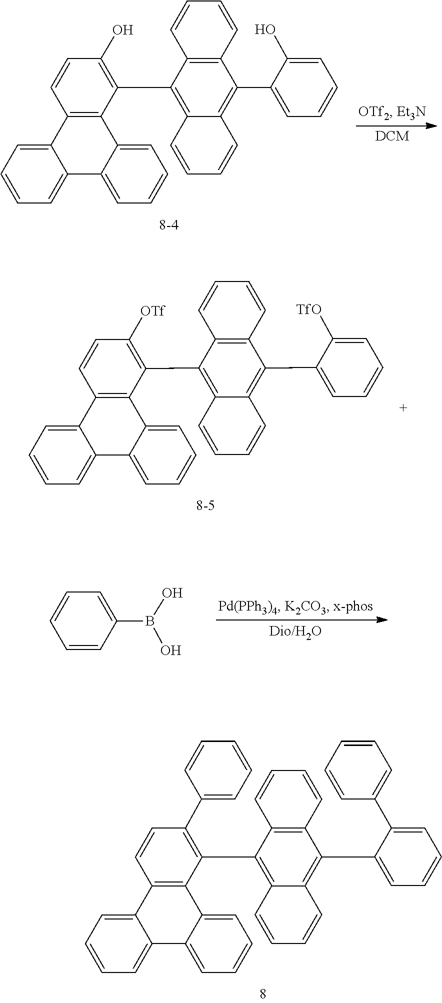

Anthraceneboronic acid (66 g, 297 mmol), compound 8-1 (95.9 g, 297 mol), potassium carbonate (82.8 g, 594 mmol), tetrakis(triphenylphosphine)palladium (10.3 g, 8.9 mmol) were added in turn to a 2 L three-necked flask. After adding 800 mL of 1,4-dioxane and 150 mL of water, the resulting mixture was heated to 90° C. under N₂ atmosphere and reacted for overnight. After cooling down to room temperature, 500 mL of water was added, then the resulting mixture separated, and the aqueous phase was extracted with DCM (300 mL*3). The organic phases were then combined, dried over anhydrous Na₂SO₄, the excess solvent was removed under reduced pressure distillation, then the resulting sample was further purified by silica gel column chromatography (eluent: PE:DCM=1:1) to yield about 108.6 g (about 87% yield) of compound 8-2. MS (ASAP)=420.5.

Compound 8-2 (108 g, 257 mmol) was added into a 2 L three-necked flask, then about 800 mL of anhydrous dichloromethane was added. After heating up to 60° C. under N₂ atmosphere, dichloromethane solution of NBS (50.3 g, 282.7 mmol) was added dropwise into the reaction system, then the reaction was reacted for 2 h in dark. After cooling down to room temperature, the excess solvent was removed under reduced pressure distillation, then the result was ultrasonically washed with 300 mL of methanol and filtrated to obtain 110 g (about 86% yield) of compound 8-3. MS (ASAP)=499.4.

Compound 8-3 (20 g, 41.3 mmol), 2-hydroxyphenylboronic acid (8.6 g, 62 mmol), potassium carbonate (11.4 g, 82.6 mmol), and tetrakis(triphenylphosphine)palladium (2.3 g, 2.1 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, 100 mL of water was added, then the result was extracted with DCM (300 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 17.1 g (about 81% yield) of compound 8-4. MS (ASAP)=512.6.

Compound 8-4 (17 g, 33.2 mmol) was added into a 500 mL three-necked flask, then about 100 mL of anhydrous dichloromethane was added. After adding triethylamine (13.4 g, 132.8 mmol) under N₂ atmosphere, the result was cooled down to about 0° C. in an ice-salt bath, trifluoromethanesulfonic anhydride (37.4 g, 132.8 mmol) was added dropwise into the above mixture, then the resulting mixture was naturally heated to room temperature and reacted for overnight. After the the reaction was completed, the reaction solution was removed to obtain crude product, then 80 mL of methanol was added. After the sonication, the result was filtrated and the residue was further washed with petroleum ether (80 mL*3) to obtained about 21.9 g (about 85% yield) of compound 8-5. MS (ASAP)=776.7.

Compound 8-5 (21.5 g, 27.7 mmol), phenylboronic acid (13.5 g, 110.8 mmol), potassium carbonate (15.3 g, 110.8 mmol), x-phos (1.3 g, 2.8 mmol), and tetrakis(triphenylphosphine)palladium (1.6 g, 1.4 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 11.4 g (about 65% yield) of compound 8. MS (ASAP)=632.8.

### Example 9

The synthetic route of the compound 9 is shown below:

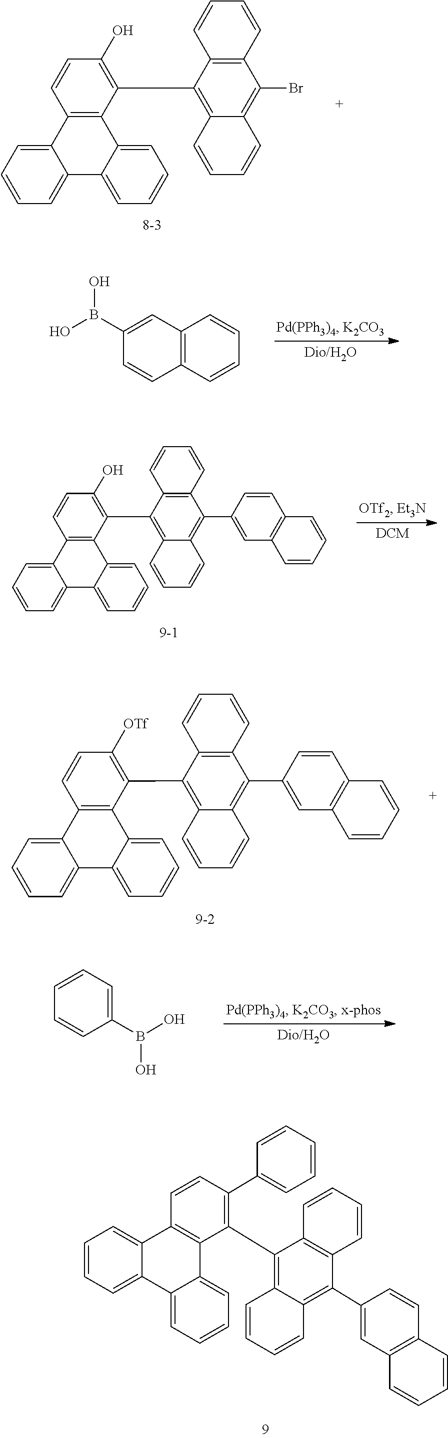

Compound 8-3 (15 g, 30 mmol), 2-naphthaleneboronic acid (5.7 g, 33 mmol), potassium carbonate (8.3 g, 60 mmol), tetrakis(triphenylphosphine)palladium (1.7 g, 1.5 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 11.2 g (about 68% yield) of compound 9-1. MS (ASAP)=546.7.

Compound 9-1 (11 g, 33.2 mmol) was added into a 500 mL three-necked flask, then about 100 mL of anhydrous dichloromethane was added. After adding triethylamine (6.7 g, 66.4 mmol) under N₂ atmosphere, the result was cooled down to about 0° C. in an ice-salt bath, trifluoromethanesulfonic anhydride (18.7 g, 66.4 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained about 20.1 g (about 89% yield) of compound 9-2. MS (ASAP)=678.7.

Compound 9-2 (20 g, 29.5 mmol), phenylboronic acid (7.2 g, 59 mmol), potassium carbonate (4.0 g, 59 mmol), x-phos (1.4 g, 2.9 mmol), and tetrakis(triphenylphosphine)palladium (1.6 g, 1.4 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 12.8 g (about 72% yield) of compound 9. MS (ASAP)=606.8.

### Example 10

The synthetic route of the compound 10 is shown below:

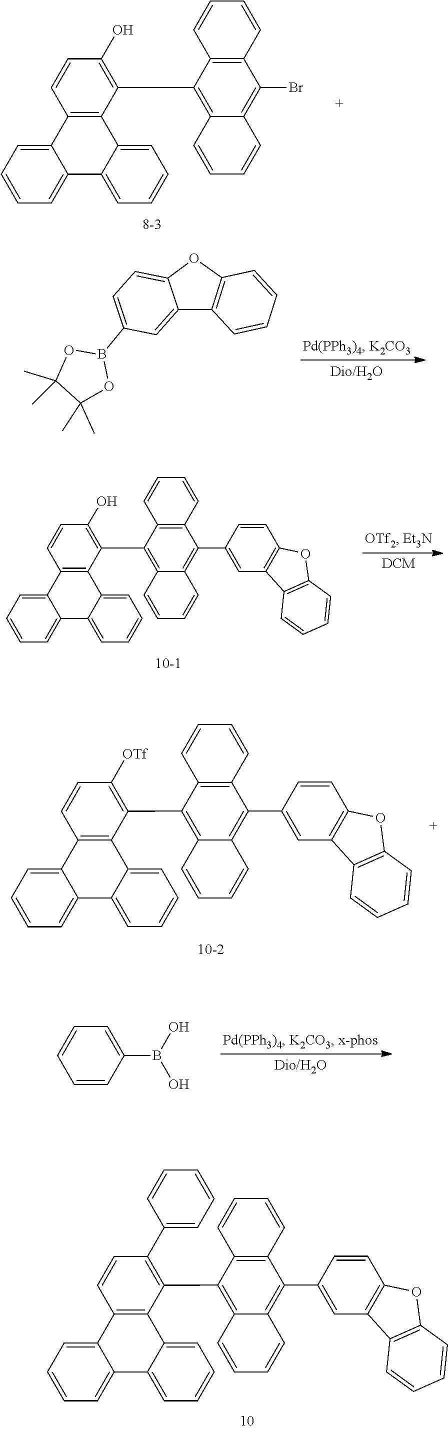 [SMILES: Oc1ccc2c3ccccc3c3ccccc3c2c1-c1c2ccccc2c(Br)c2ccccc12 ; CC1(C)OB(c2ccc3oc4ccccc4c3c2)OC1(C)C ; Oc1ccc2c3ccccc3c3ccccc3c2c1-c1c2ccccc2c(-c2ccc3oc4ccccc4c3c2)c2ccccc12 ; O=S(=O)(Oc1ccc2c3ccccc3c3ccccc3c2c1-c1c2ccccc2c(-c2ccc3oc4ccccc4c3c2)c2ccccc12)C(F)(F)F ; OB(O)c1ccccc1 ; c1ccc(-c2ccc3c4ccccc4c4ccccc4c3c2-c2c3ccccc3c(-c3ccc4oc5ccccc5c4c3)c3ccccc23)cc1]

Compound 8-3 (15 g, 30 mmol), dibenzofuran-2-boronate (9.7 g, 33 mmol), potassium carbonate (8.3 g, 60 mmol), and tetrakis(triphenylphosphine)palladium (1.7 g, 1.5 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 13.7 g (about 78% yield) of compound 10-1. MS (ASAP)=586.7.

Compound 10-1 (13.5 g, 23 mmol) was added into a 500 mL three-necked flask, then about 150 mL of anhydrous dichloromethane was added. After adding triethylamine (4.6 g, 46 mmol) under N₂ atmosphere, the result was cooled down to about 0° C. in an ice-salt bath, trifluoromethanesulfonic anhydride (13 g, 46 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained about 14.7 g (about 89% yield) of compound 10-2. MS (ASAP)=718.8.

Compound 10-2 (14.5 g, 20.1 mmol), phenylboronic acid (4.9 g, 40.2 mmol), potassium carbonate (5.5 g, 40.2 mmol), x-phos (0.95 g, 2.0 mmol), and tetrakis(triphenylphosphine)palladium (1.1 g, 1 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 9.2 g (about 71% yield) of compound 10. MS (ASAP)=646.8.

### Example 11

The synthetic route of compound 11 is shown below:

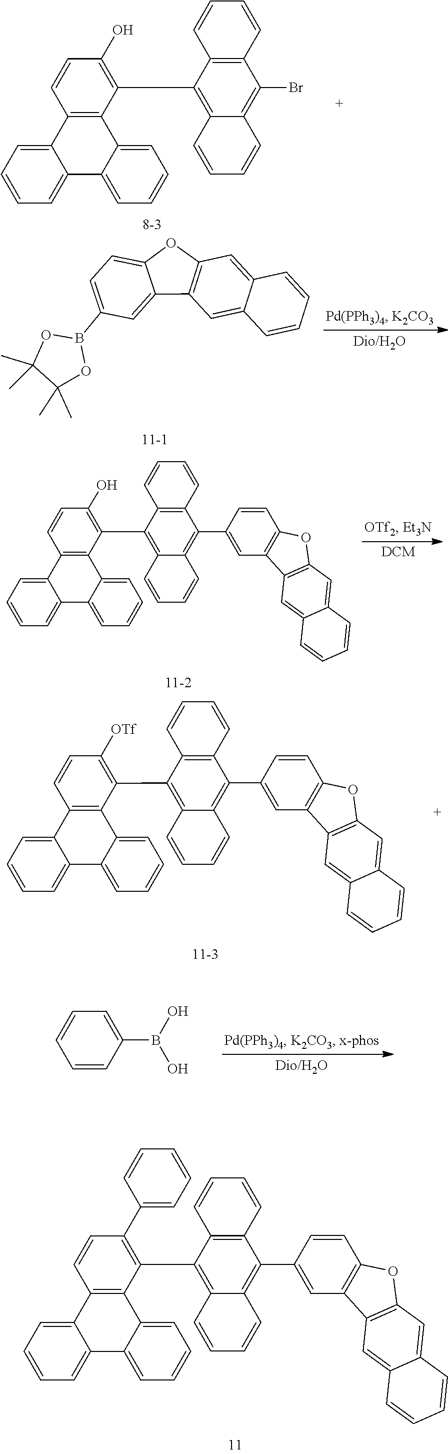

Compound 8-3 (15 g, 30 mmol), compound 11-1 (11.4 g, 33 mmol), potassium carbonate (8.3 g, 60 mmol), tetrakis(triphenylphosphine)palladium (1.7 g, 1.5 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 14.3 g (about 75% yield) of compound 11-2. MS (ASAP)=636.8.

Compound 11-2 (14 g, 22 mmol) was added into a 500 mL three-necked flask, then about 150 mL of anhydrous dichloromethane was added. After adding triethylamine (4.4 g, 44 mmol) under N₂ atmosphere, the result was cooled down to about 0° C. in an ice-salt bath, trifluoromethanesulfonic anhydride (12.4 g, 44 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained about 14.5 g (about 86% yield) of compound 11-3. MS (ASAP)=768.8.

Compound 11-3 (14.5 g, 20.1 mmol), phenylboronic acid (4.9 g, 40.2 mmol), potassium carbonate (5.5 g, 40.2 mmol), x-phos (0.95 g, 2.0 mmol), and tetrakis(triphenylphosphine)palladium (1.1 g, 1 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 10.2 g (about 73% yield) of compound 11. MS (ASAP)=696.9.

### Example 12

The synthetic route of the compound 12 is shown below:

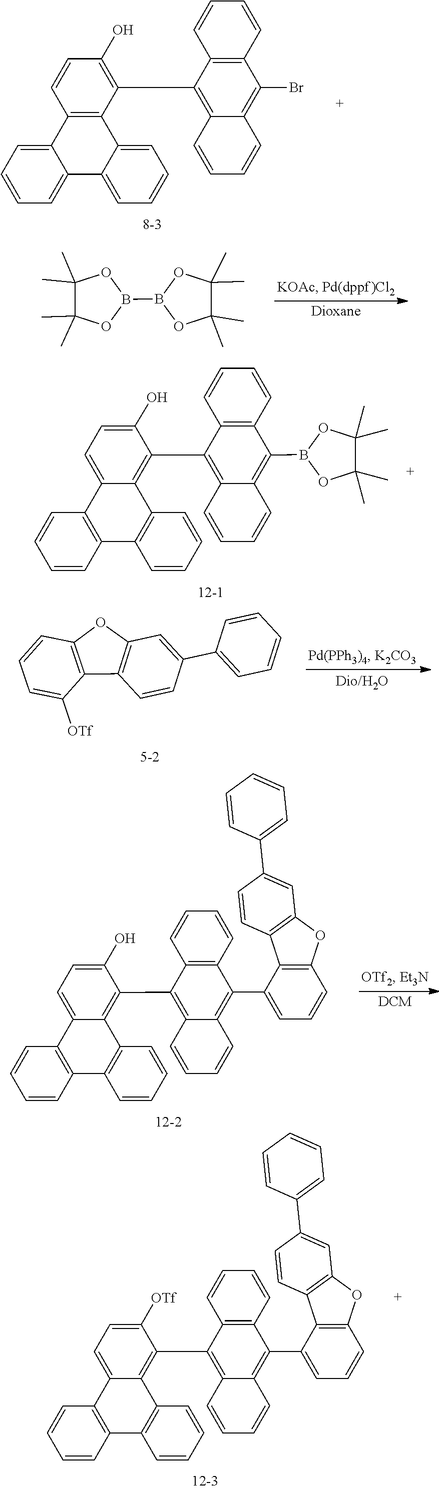

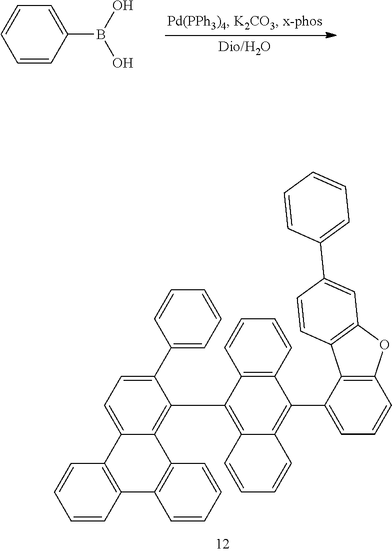 [SMILES: OB(O)c1ccccc1 ; c1ccc(-c2ccc3c(c2)oc2cccc(-c4c5ccccc5c(-c5c(-c6ccccc6)ccc6c7ccccc7c7ccccc7c56)c5ccccc45)c23)cc1]

Compound 8-3 (15 g, 30 mmol), bis(pinacolato)diboron (11.4 g, 45 mmol), potassium acetate (4.4 g, 45 mmol), Pd(dppf)Cl₂ (1.1 g, 1.5 mmol) were added in turn to a 250 mL three-necked flask. After adding about 100 mL of anhydrous 1,4-dioxane, the resulting mixture was heated to 110° C. under N₂ atmosphere and reacted for 4 h. After the reaction was completed, the excess solvent was removed under reduced pressure distillation, then the resulting sample was further purified by silica gel column chromatography (eluent: PE:DCM=5:1) to yield 13.9 g (about 85% yield) of compound 12-1. MS (ASAP)=546.5.

Compound 12-1 (13.9 g, 25.4 mmol), compound 5-2 (10 g, 25.4 mmol), potassium carbonate (7 g, 50.8 mmol), and tetrakis(triphenylphosphine)palladium (1.5 g, 1.3 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 12.3 g (about 73% yield) of compound 12-2. MS (ASAP)=662.8.

Compound 12-2 (12 g, 18.1 mmol) was added into a 500 mL three-necked flask, then about 150 mL of anhydrous dichloromethane was added. After adding triethylamine (3.7 g, 36.2 mmol) under N₂ atmosphere, the result was cooled down to about 0° C. in an ice-salt bath, trifluoromethanesulfonic anhydride (10.2 g, 36.2 mmol) was added dropwise into the above mixture, then the resulting mixture was naturally heated to room temperature and reacted for overnight. After the reaction was completed, the reaction solution was removed to obtain crude product, then 80 mL of methanol was added. After the sonication, the result was filtrated and the residue was further washed with petroleum ether (80 mL*3) to obtained 11.5 g (about 80% yield) of compound 12-3. MS (ASAP)=794.8.

Compound 12-3 (11.5 g, 14.5 mmol), phenylboronic acid (3.5 g, 29 mmol), potassium carbonate (4.0 g, 29 mmol), x-phos (0.7 g, 1.5 mmol), and tetrakis(triphenylphosphine)palladium (0.8 g, 0.7 mmol) were added in turn to a 250 mL three-necked flask. After adding 100 mL of 1,4-dioxane and 20 mL of water, the resulting mixture was heated to 90° C. under N₂ atmosphere and reacted for overnight. After cooling down to room temperature, the result was diluted with 60 mL of water, then extracted with DCM (80 mL*3). The organic phases were then combined, dried over anhydrous Na₂SO₄, the excess solvent was removed under reduced pressure distillation, then the resulting sample was further purified by silica gel column chromatography (eluent: petroleum ether) to yield 7.7 g (about 73% yield) of compound 12. MS (ASAP)=722.9.

### Example 13

The synthetic route of the compound 13 is shown below:

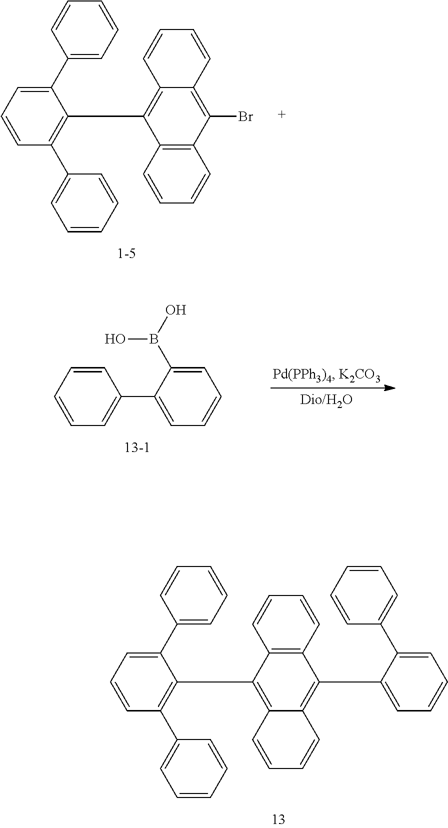

Compound 1-5 (22 g, 45 mmol), 2-biphenylboronic acid (10.7 g, 54 mmol), tetrakis(triphenylphosphine)palladium (2.5 g, 2.3 mmol), and potassium carbonate (12.4 g, 90 mmol) were added in turn to a 500 mL three-necked flask. After adding 240 mL of 1,4-dioxane and 40 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 20 g (about 78% yield) of compound 13. MS (ASAP)=558.7.

### Example 14

The synthetic route of the compound 14 is shown below:

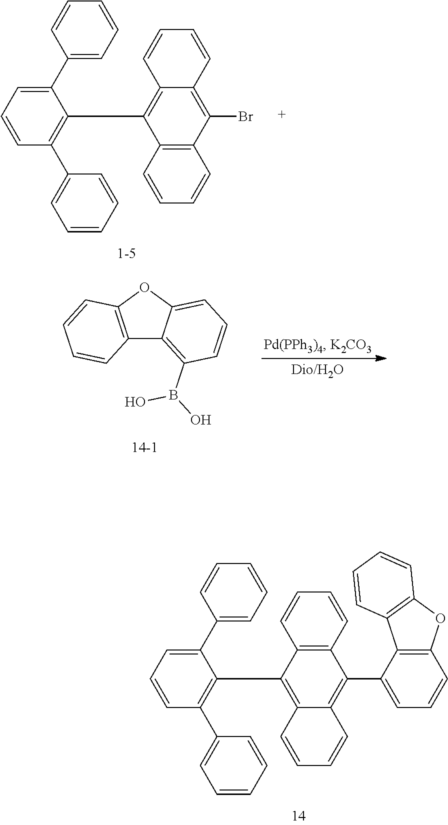 [SMILES: Brc1c2ccccc2c(-c2c(-c3ccccc3)cccc2-c2ccccc2)c2ccccc12 ; OB(O)c1cccc2oc3ccccc3c12 ; c1ccc(-c2cccc(-c3ccccc3)c2-c2c3ccccc3c(-c3cccc4oc5ccccc5c34)c3ccccc23)cc1]

Compound 1-5 (22 g, 45 mmol), 1-dibenzofuranylboronic acid (11.4 g, 54 mmol), tetrakis(triphenylphosphine)palladium (2.5 g, 2.3 mmol), and potassium carbonate (12.4 g, 90 mmol) were added in turn to a 500 mL three-necked flask. After adding 240 mL of 1,4-dioxane and 40 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 20 g (about 72% yield) of compound 14. MS (ASAP)=572.7.

### Example 15

The synthetic route of the compound 15 is shown below:

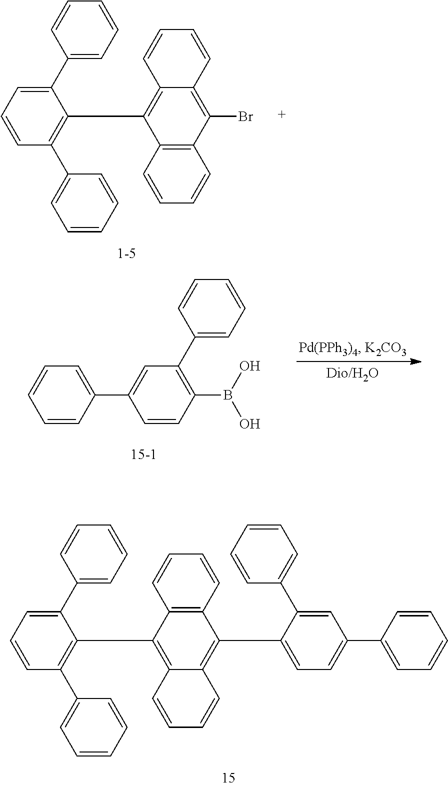

Compound 1-5 (22 g, 45 mmol), 1,3-m-diphenyl-4-boronic acid (14.8 g, 54 mmol), tetrakis(triphenylphosphine)palladium (2.5 g, 2.3 mmol), and potassium carbonate (12.4 g, 90 mmol) were added in turn to a 500 mL three-necked flask. After adding 240 mL of 1,4-dioxane and 40 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 20 g (about 66% yield) of compound 15. MS (ASAP)=634.8.

### Example 16

The synthetic route for compound 16 is shown below:

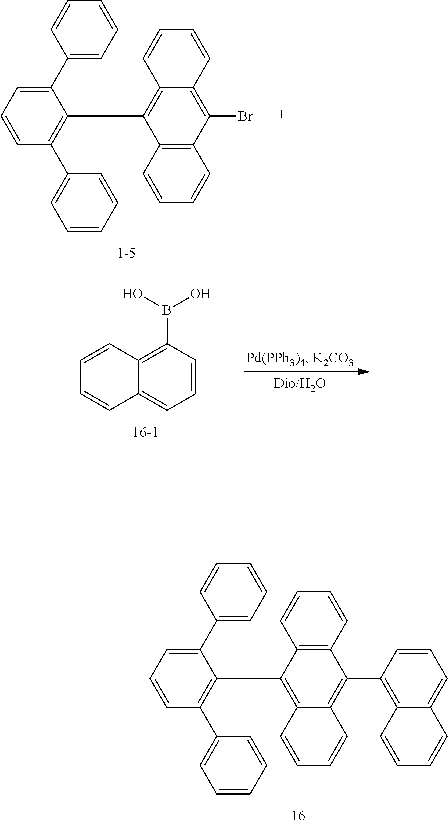

### Comparative Example 1:

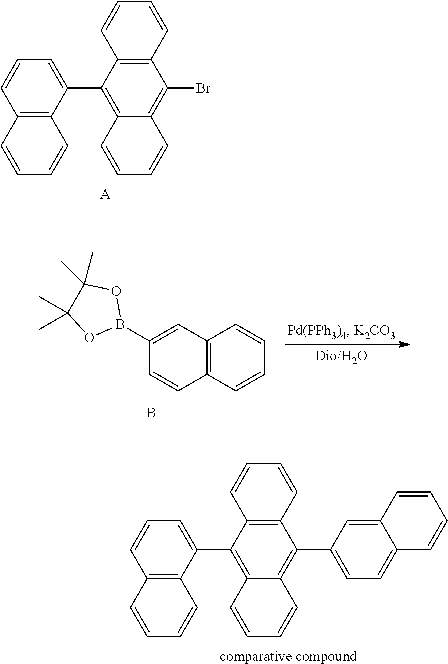

Compound A (19 g, 49.6 mmol), compound B (12.6 g, 49.6 mmol), tetrakis(triphenylphosphine)palladium (2.8 g, 2.5 mmol), and potassium carbonate (3.7 g, 99.2 mmol) were added in turn to a 500 mL three-necked flask. After adding 200 mL of 1,4-dioxane and 40 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 18.1 g (about 85% yield) of comparative compound 1. MS (ASAP)=430.6.

### Comparative Example 2:

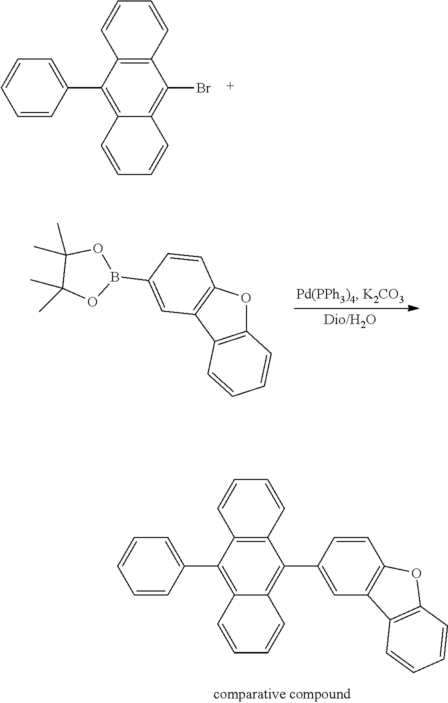 [SMILES: Brc1c2ccccc2c(-c2ccccc2)c2ccccc12 ; CC1(C)OB(c2ccc3oc4ccccc4c3c2)OC1(C)C ; c1ccc(-c2c3ccccc3c(-c3ccc4oc5ccccc5c4c3)c3ccccc23)cc1]

9-Bromo-10-phenylanthracene (15 g, 45 mmol), dibenzofuran-2-boronate (13.2 g, 45 mmol), tetrakis(triphenylphosphine)palladium (2.5 g, 2.2 mmol), and potassium carbonate (12.4 g, 90 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, 300 mL of water was added, then the result was extracted with DCM (300 mL*3). The organic phases were then combined, dried over anhydrous Na₂SO₄, the excess solvent was removed under reduced pressure distillation, then the residue was recrystallized with toluene and n-hexane (volume ratio: 10:3) to yield 16.8 g (about 89% yield) of comparative compound 2. MS (ASAP)=420.5.

### Comparative Example 3:

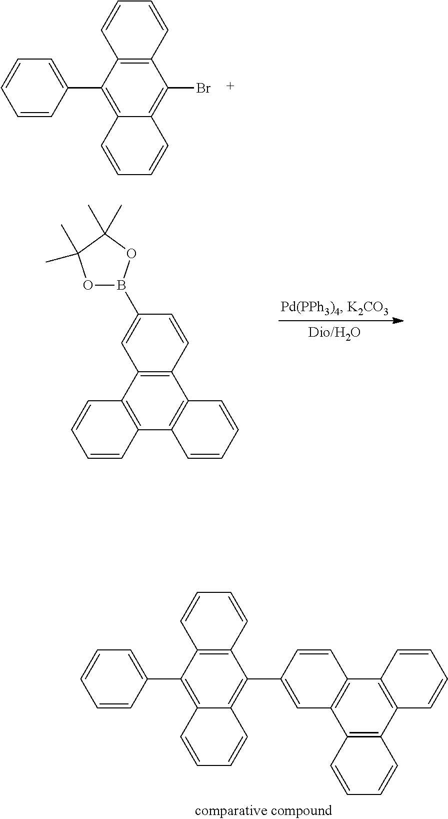

9-Bromo-10-phenylanthracene (15 g, 45 mmol), tritylene-2-boronate (15.9 g, 45 mmol), tetrakis(triphenylphosphine)palladium (2.5 g, 2.2 mmol), and potassium carbonate (12.4 g, 90 mmol) were added in turn to a 500 mL three-necked flask. After adding 150 mL of 1,4-dioxane and 30 mL of water, the resulting mixture was heated to 90° C. under N₂ atmosphere and reacted for overnight. After cooling down to room temperature, 300 mL of water was added, then the result was extracted with DCM (300 mL*3). The organic phased were then combined, dried over anhydrous Na₂SO₄, the excess solvent was removed under reduced pressure distillation, then the residue was recrystallized with toluene and n-hexane (volume ratio: 10:3) to yield 17.9 g (about 83% yield) of comparative compound 3. MS (ASAP)=480.6.

The energy level of the organic material can be calculated by quantum computation, for example, using TD-DFT (time-dependent density functional theory) by Gaussian09W (Gaussian Inc.), the specific simulation methods of which can be found in WO2011141110. Firstly, the molecular geometry is optimized by semi-empirical method “Ground State/Semi-empirical/Default Spin/AM1” (Charge 0/Spin Singlet), and then the energy structure of organic molecules is calculated by TD-DFT (time-dependent density functional theory) “TD-SCF/DFT/Default Spin/B3PW91” and the basis set “6-31G (d)” (Charge 0/Spin Singlet). The HOMO and LUMO levels are calculated using the following calibration formula, where S₁, T₁, T₂ and resonant factor f(S₁) are used directly.

HOMO(eV)=((HOMO(G)×27.212)−0.9899)/1.1206

LUMO(eV)=((LUMO(G)×27.212)−2.0041)/1.385

Where HOMO(G) and LUMO(G) are the direct calculation results of Gaussian 09W, in units of Hartree. The results are shown in Table 1 below:

TABLE 1

| Material | HOMO | LUMO | f(S₁) | T₁ | T₂ | S₁ | ΔE_{S} |
|---|---|---|---|---|---|---|---|
| compound 1 | −5.51 | −2.72 | 0.1889 | 1.64 | 3.16 | 3.08 | 1.44 |
| compound 2 | −5.50 | −2.73 | 0.2333 | 1.64 | 2.79 | 3.06 | 1.42 |
| compound 3 | −5.53 | −2.74 | 0.2146 | 1.64 | 2.37 | 3.07 | 1.43 |
| compound 4 | −5.50 | −2.74 | 0.1731 | 1.62 | 3.01 | 3.04 | 1.42 |
| compound 5 | −5.56 | −2.76 | 0.1618 | 1.64 | 2.92 | 3.07 | 1.43 |
| compound 6 | −5.51 | −2.73 | 0.1607 | 1.63 | 3.04 | 3.04 | 1.41 |
| compound 7 | −5.50 | −2.73 | 0.1744 | 1.63 | 3.02 | 3.05 | 1.42 |
| compound 8 | −5.49 | −2.74 | 0.1731 | 1.61 | 2.73 | 3.02 | 1.42 |
| compound 9 | −5.49 | −2.74 | 0.2225 | 1.61 | 2.65 | 3.03 | 1.42 |
| compound 10 | −5.51 | −2.74 | 0.2191 | 1.62 | 2.72 | 3.04 | 1.43 |
| compound 11 | −5.53 | −2.76 | 0.2512 | 1.62 | 2.37 | 3.04 | 1.42 |
| compound 12 | −5.56 | −2.79 | 0.2020 | 1.61 | 2.72 | 3.02 | 1.41 |
| compound 13 | −5.48 | −2.72 | 0.3008 | 1.62 | 3.20 | 3.05 | 1.43 |
| compound 14 | −5.55 | −2.76 | 0.1520 | 1.64 | 3.15 | 3.07 | 1.43 |
| compound 15 | −5.49 | −2.72 | 0.1919 | 1.62 | 3.13 | 3.04 | 1.42 |
| compound 16 | −5.51 | −2.72 | 0.1579 | 1.64 | 2.64 | 3.09 | 1.45 |
| comparative | −5.56 | −2.70 | 0.0006 | 1.68 | 2.64 | 3.50 | 1.83 |
| compound 1 | | | | | | | |
| comparative | −5.56 | −2.70 | 0.1824 | 1.68 | 3.22 | 3.18 | 1.51 |
| compound 2 | | | | | | | |
| comparative | −5.55 | −2.69 | 0.2105 | 1.68 | 2.79 | 3.18 | 1.50 |
| compound 3 | | | | | | | |

### Device Example

Materials used in each layer of an OLED device:

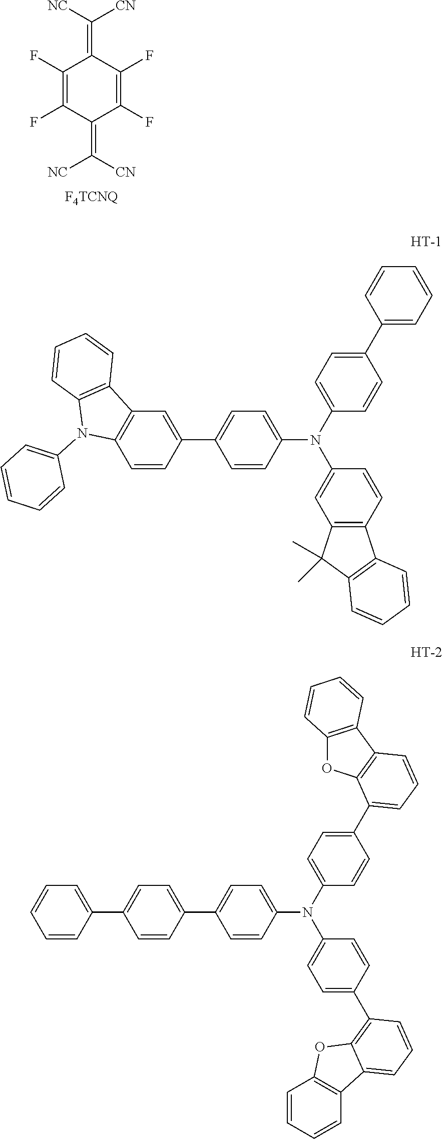 [SMILES: N#CC(C#N)=c1c(F)c(F)c(=C(C#N)C#N)c(F)c1F ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(-c3ccc(N(c4ccc(-c5cccc6c5oc5ccccc56)cc4)c4ccc(-c5cccc6c5oc5ccccc56)cc4)cc3)cc2)cc1]

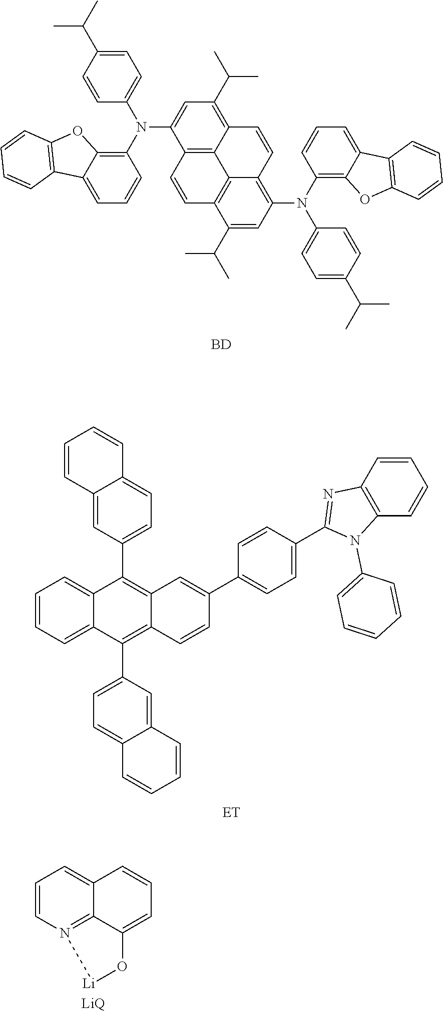

The OLEDs were fabricated based on the device structure of ITO/HI(10 nm)/HT-1(50 nm)/HT-2(10 nm)/BH:BD(25 nm)/ET:LiQ(30 nm)/LiQ(1 nm)/A1(100 nm), and the preparation procedures are as follows.
a. Cleaning of the conductive glass substrate: prior to first-time use, the substrates are washed with various solvents (such as chloroform, ketone, or isopropyl alcohol), and then treated with UV and ozone.
b. The resultant ITO substrate was mounted on a vacuum deposition apparatus in high vacuum (1×10⁻⁶ mbar), the weight ratio of HT-1 and F4TCNQ was controlled to be 98:2 to form a hole-injection layer (HIL) having a thickness of 10 nm, then compound HT-1 and compound HT-2 were vacuum-deposited sequentially on the hole-injection layer to form two hole transport layers having a thickness of 50 nm and 10 nm, respectively. Then the weight ratio of BH and BD was controlled to be 97:3 to form a light-emitting layer having a thickness of 25 nm. Subsequently, compound ET and Liq were placed in two different evaporation units, and co-deposited on the light-emitting layer at a weight ratio of 50:50 to form an electron-transport layer having a thickness of 30 nm. LiQ was then deposited on the electron-transport layer to form an electron-injection layer having a thickness of 1 nm, and Al was deposited on the electron-injection layer to form a cathode having a thickness of 100 nm.
d. Encapsulation: encapsulating the device in a nitrogen-regulated glove box with UV curable resin.

All devices have the same embodiments except for the light-emitting layer, which uses a different compound or mixture as the blue host materials. The current-voltage (J-V) characteristics of each OLED were studied. The current efficiency, device lifetime and the external quantum efficiency were summarized in Table 2, where the lifetime value is the relative value of the devices with the comparative compound 2.

TABLE 2

| | | | Device |
|---|---|---|---|
| | | | lifetime@50 |
| | | EQE | mA/cm² |
| | blue host material (BH) | (%) | (LT95, %) |
| Device Example 1 | compound 2 | 7.2 | 110% |
| Device Example 1a | compound 2 + comparative | 7.1 | 115% |
| | compound 2 (1:1) | | |
| Device Example 2 | compound 4 | 7.9 | 109% |
| Device Example 2a | compound 4 + comparative | 7.7 | 116% |
| | compound 2 (1:1) | | |
| Device Example 3 | compound 6 | 7.3 | 115% |
| Device Example 3a | compound 6 + comparative | 7.6 | 120% |
| | compound 2 (1:1) | | |
| Device Example 4 | compound 7 | 7.9 | 106% |
| Device Example 4a | compound 7 + comparative | 7.8 | 115% |
| | compound 2 (1:1) | | |
| Device Example 5 | compound 14 | 7.8 | 118% |
| Device Example 5a | compound 14 + comparative | 7.7 | 116% |
| | compound 2 (1:1) | | |
| Device Example 6 | compound 16 | 7.6 | 107% |
| Device Example 6a | compound 16 + comparative | 7.7 | 112% |
| | compound 2 (1:1) | | |
| Device Example 7 | compound 13 | 7.8 | 92% |
| Device Example 7a | compound 13 + comparative | 7.6 | 111% |
| | compound 2 (1:1) | | |
| Device Example 8 | compound 15 | 7.7 | 90% |
| Device Example 8a | compound 15 + comparative | 7.67 | 106% |
| | compound 2 (1:1) | | |
| Comparative | comparative compound 1 | 6.7 | 74% |
| Example 1 | | | |
| Comparative | comparative compound 2 | 7.4 | 100% |
| Example 2 | | | |
| Comparative | comparative compound 3 | 7.3 | 89% |
| Example 3 | | | |

Compared with the comparative examples 1-3, for the organic compound used in the device examples 1-8, the introduction of the bulky groups at 9 and 10-positions of the anthracence compound provides a large steric hindrance, which can effectively reduces the effect of O₂ on the carbon atom at 9 and 10-positions of anthracene, thereby prolong the device lifetime.

In addition, the use of the mixture in device examples 1a-8a also enhances the device lifetime.

It will be understood that the application of the present disclosure is not limited to the foregoing examples, and may be improved or transformed in accordance with the foregoing description to one of ordinary skill in the art, all these modifications and improvements are within the scope of the present disclosure.

What is claimed is:

1. An organic compound, comprising a structure of formula (I): 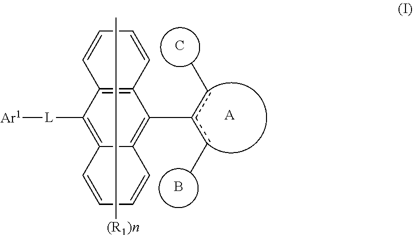
wherein:
A, B, C are the same or different, and are independently selected from the group consisting of a substituted/unsubstituted fused aromatic or heteroaromatic ring structure containing 5 to 30 ring atoms, B and C are each linked to A at the adjacent position of the linkage site between A and anthracene unit, and B and C are fused with A to form a ring, respectively;
Ar¹ is selected from a substituted/unsubstituted fused aromatic or heteroaromatic ring structure containing 8 to 40 ring atoms;
L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof;
R₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group;
n is integer from 0 to 8, and the adjacent two R₁s are fused to form a ring.

2. The organic compound according to claim 1, comprising a structure of formulas (II-a)-(II-f): 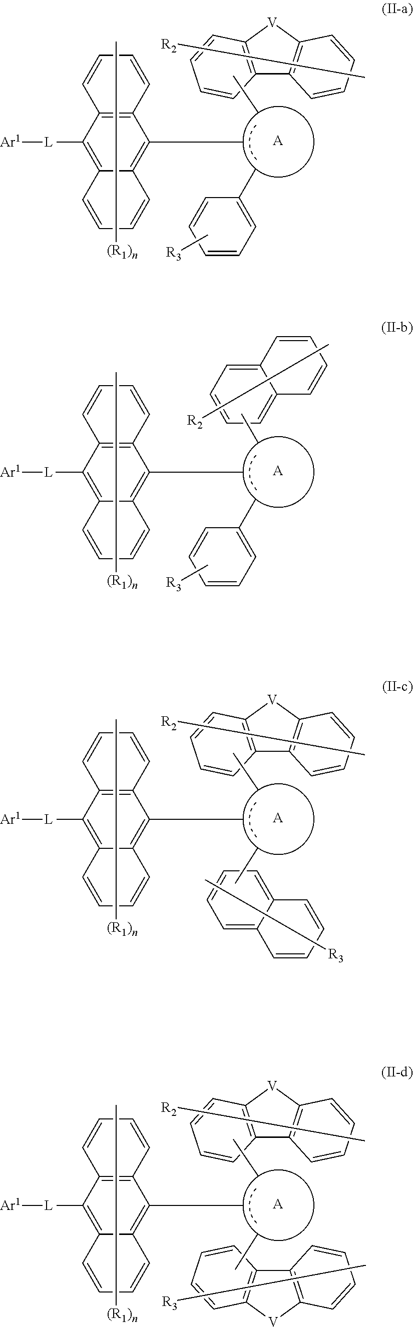 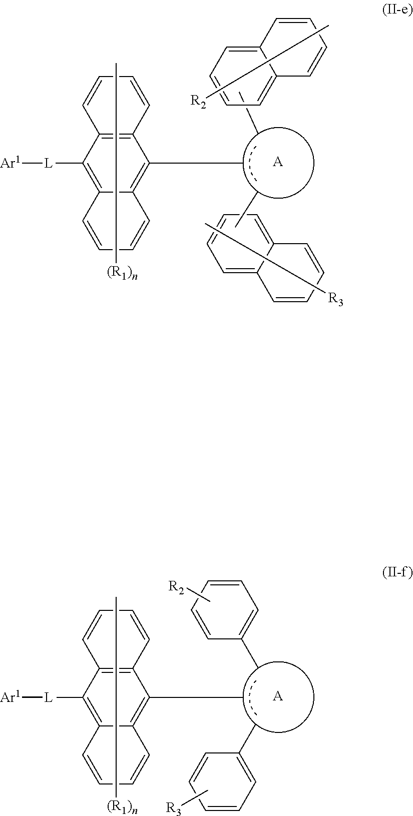
wherein:
V, in multiple occurrences, is independently selected from B(R₁₀), C(═O), N(R₁₀), C(R₁₀R₁₁), O, S, P, P═O, or P═S;
each of R₁, R₂, R₃, R₁₀, and R₁₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group;
each A is independently selected from the group consisting of a substituted/unsubstituted fused aromatic or heteroaromatic ring structure containing 5 to 30 ring atoms; Ar¹ is selected from a substituted/unsubstituted fused aromatic or heteroaromatic ring structure containing 8 to 40 ring atoms; L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof; n is integer from 0 to 8.

3. The organic compound according to claim 1, comprising a structure of one of the formulas (III-a)-(III-c): 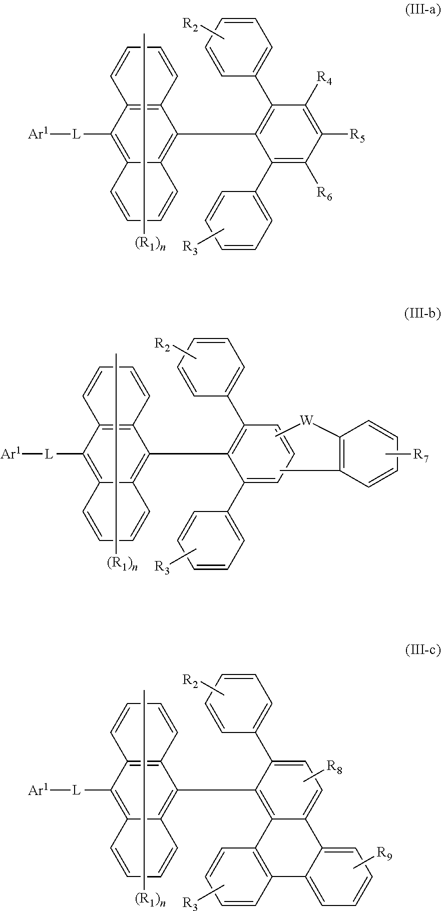
wherein:
W, in multiple occurrences, is independently selected from B(R₃₀), C(═O), N(R₃₁), O, S, P, P═O, or P═S;
each of R₁-R₉, R₃₀, and R₃₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; while R₄ and R₆ are independently fused with R₅ to form a ring;
Ar¹ is selected from a substituted/unsubstituted fused aromatic or heteroaromatic ring structure containing 8 to 40 ring atoms; L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof; n is integer from 0 to 8.

4. The organic compound according to claim 2, comprising a structure of one of the formulas (III-a)-(III-c): 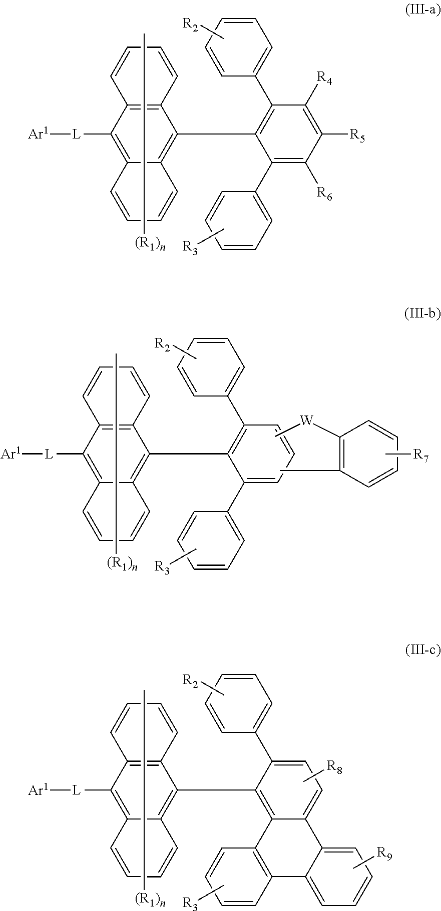
wherein:
W, in multiple occurrences, is independently selected from B(R₃₀), C(═O), N(R₃₁), O, S, P, P═O, or P═S;
each of R₁-R₉, R₃₀, and R₃₁ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; while R₄ and R₆ are independently fused with R₅ to form a ring;
Ar¹ is selected from a substituted/unsubstituted fused aromatic or heteroaromatic ring structure containing 8 to 40 ring atoms; L is a linking group selected from the group consisting of a single bond, a C₆-C₃₀ arylene group, a C₂-C₃₀ heteroaromatic group, a C₃-C₃₀ aliphatic ring, a C₆-C₃₀ fused aromatic ring, and any combination thereof; n is integer from 0 to 8.

5. The organic compound according to claim 1, wherein each Ar¹ is the same or different, and is independently selected from a substituted/unsubstituted naphthalene, anthracene, phenanthrene, fluoranthene, pyrene, fluorene, pyridine, dibenzofuran, etc., where the substituents of the or one of the following structures: 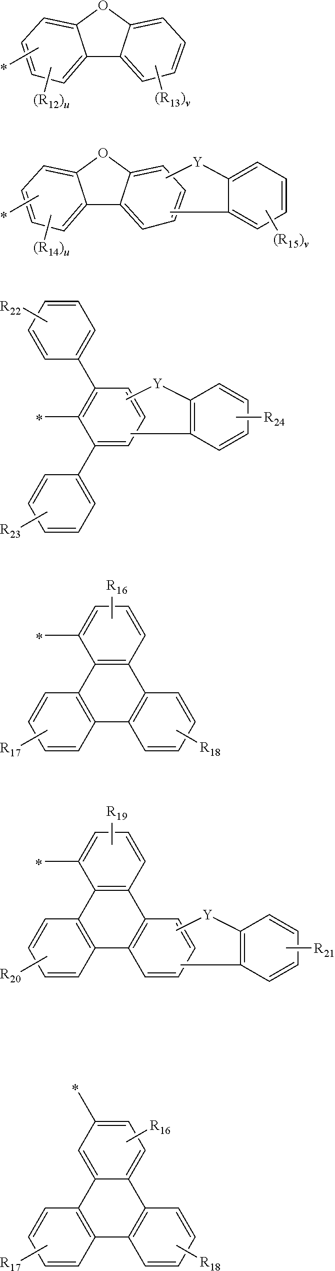
wherein:
Y, in multiple occurrences, is independently selected from B(R₂₅), C(═O), N(R₂₆), C(R₂₅R₂₆), O, S, P, P═O, or P═S;
* denotes a linkage site;
each of R₁₂ to R₂₆ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; u, v are integers from 0 to 8, and when a plurality of substituents are present at the same time, the adjacent two substituents are fused to form a ring.

6. The organic compound according to claim 2, wherein each Ar¹ is the same or different, and is independently selected from a substituted/unsubstituted naphthalene, anthracene, phenanthrene, fluoranthene, pyrene, fluorene, pyrrole, furan, thiophene, pyridine, cyclopentadiene, dibenzofuran or one of the following structures: 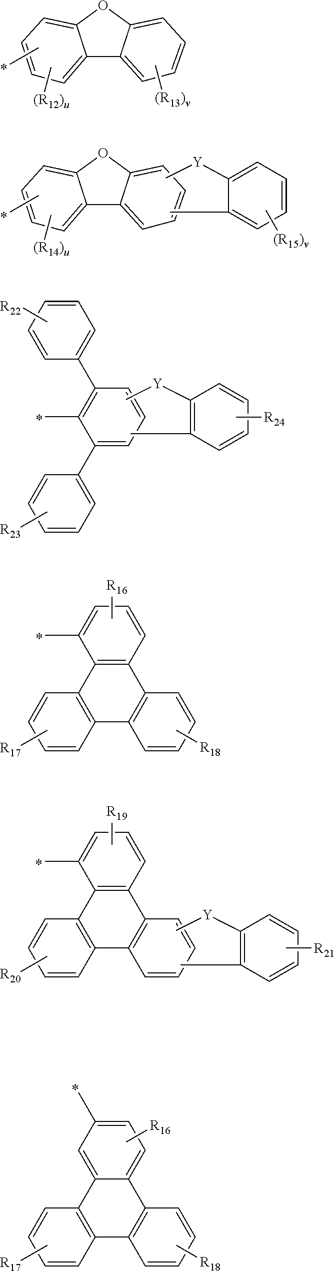
wherein:
Y, in multiple occurrences, is independently selected from B(R₂₅), C(═O), N(R₂₆), C(R₂₅R₂₆), O, S, P, P═O, or P═S;
* denotes a linkage site;
each of R₁₂ to R₂₆ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; u, v are integers from 0 to 8, and when a plurality of substituents are present at the same time, the adjacent two substituents are fused to form a ring.

7. The organic compound according to claim 3, wherein each Ar¹ is the same or different, and is independently selected from a substituted/unsubstituted naphthalene, anthracene, phenanthrene, fluoranthene, pyrene, fluorene, pyrrole, furan, thiophene, pyridine, cyclopentadiene, dibenzofuran or one of the following structures: 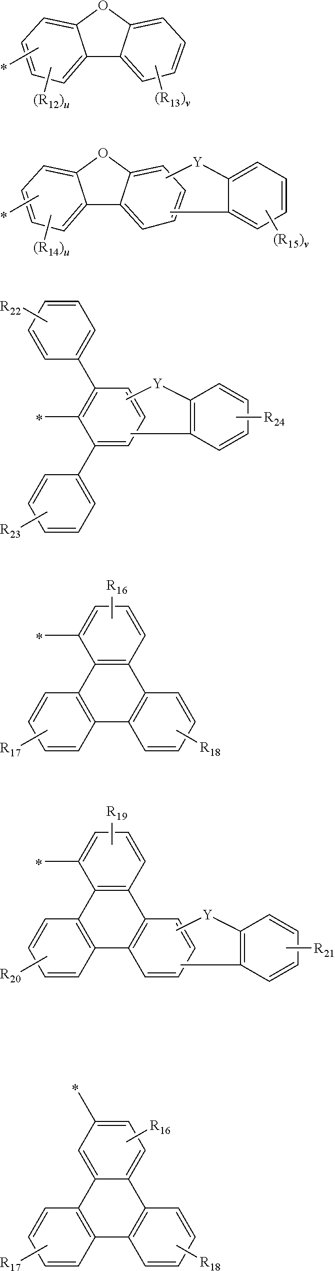
wherein:
Y, in multiple occurrences, is independently selected from B(R₂₅), C(═O), N(R₂₆), C(R₂₅R₂₆), O, S, P, P═O, or P═S;
* denotes a linkage site;
each of R₁₂ to R₂₆ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; u, v are integers from 0 to 8, and when a plurality of substituents are present at the same time, the adjacent two substituents are fused to form a ring.

8. The organic compound according to claim 4, wherein each Ar¹ is the same or different, and is independently selected from a substituted/unsubstituted naphthalene, anthracene, phenanthrene, fluoranthene, pyrene, fluorene, pyrrole, furan, thiophene, pyridine, cyclopentadiene, dibenzofuran or one of the following structures: 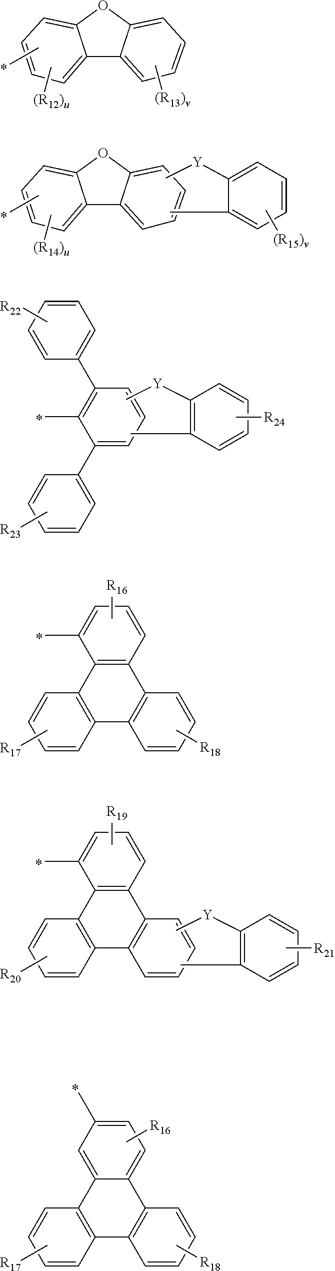
wherein:
Y, in multiple occurrences, is independently selected from B(R₂₅), C(═O), N(R₂₆), C(R₂₅R₂₆), O, S, P, P═O, or P═S;
* denotes a linkage site;
each of R₁₂ to R₂₆ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group; u, v are integers from 0 to 8, and when a plurality of substituents are present at the same time, the adjacent two substituents are fused to form a ring.

9. The organic compound according to claim 1, wherein L is the same or different, and is independently selected from a single bond, one or combinations of more than one of the following groups: 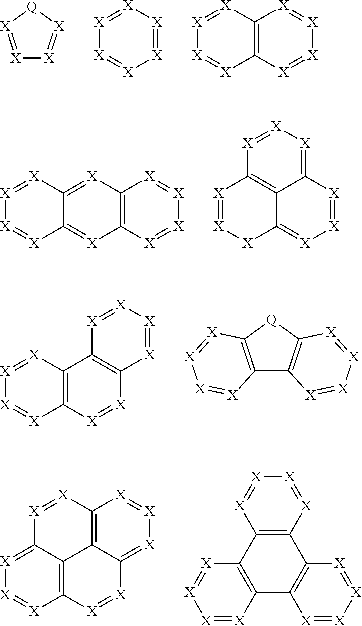
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

10. The organic compound according to claim 2, wherein L is the same or different, and is independently selected from a single bond, one or combinations of more than one of the following groups: 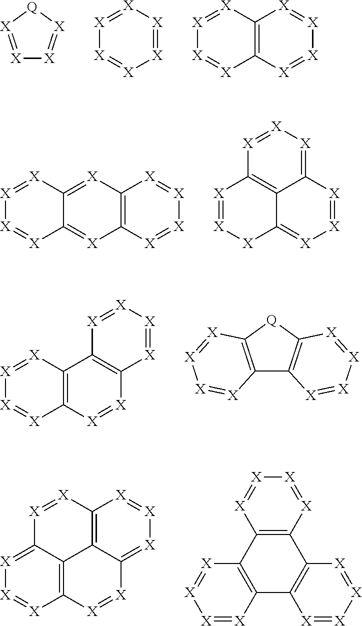
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

11. The organic compound according to claim 3, wherein L is the same or different, and is independently selected from a single bond, one or combinations of more than one of the following groups: 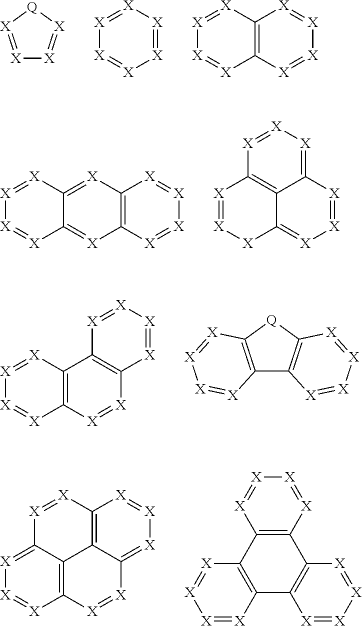
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

12. The organic compound according to claim 4, wherein L is the same or different, and is independently selected from a single bond, one or combinations of more than one of the following groups: 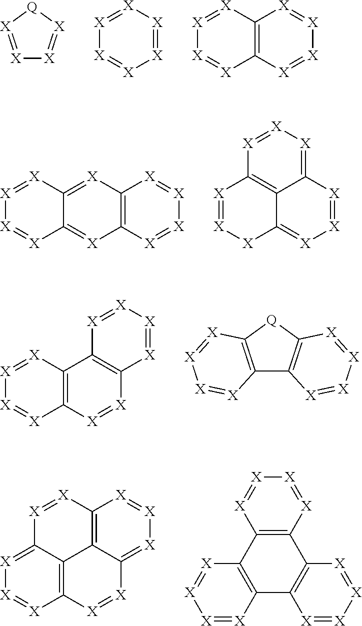
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

13. The organic compound according to claim 5, wherein L is the same or different, and is independently selected from a single bond, one or combinations of more than one of the following groups: 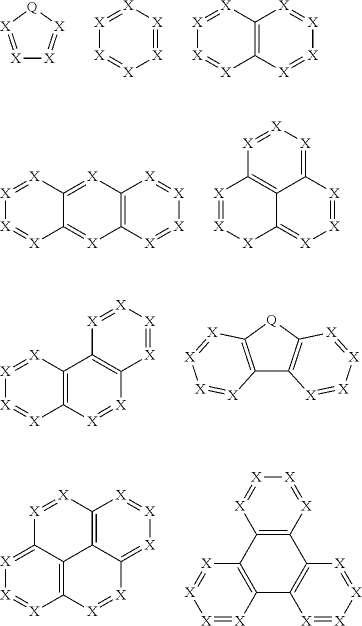
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

14. The organic compound according to claim 6, wherein L is the same or different, and is independently selected from a single bond, one or combinations of more than one of the following groups: 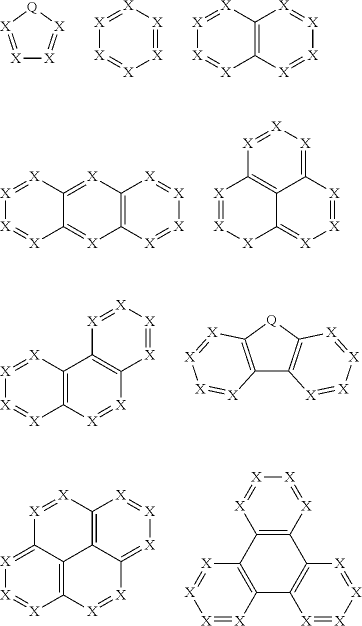
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

15. The organic compound according to claim 7, wherein L is the same or different, and is independently selected from a single bond, one or combinations of more than one of the following groups: 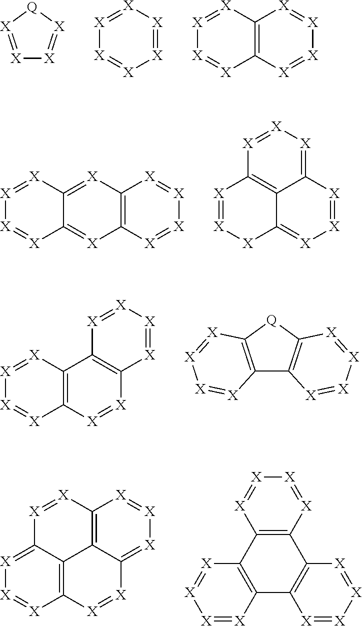
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

16. The organic compound according to claim 8, wherein L is the same or different, and is independently selected from a single bond, one or combinations of more than one of the following groups: 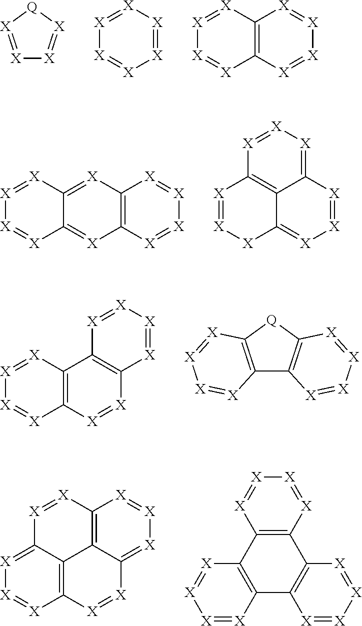
wherein:
X, in multiple occurrences, is independently selected from C(R₂₇) or N;
Q, in multiple occurrences, is independently selected from B(R₂₈), C(═O), N(R₂₉), O, S, P, P═O, or P═S;
each of R₂₇ to R₂₉ is a substituent, and at each occurrence is independently selected from the group consisting of a C₁-C₂₀ linear alkyl group, a C₁-C₂₀ linear alkoxy group, a C₁-C₂₀ linear thioalkoxy group, a C₃-C₂₀ branched/cyclic alkyl group, a C₃-C₂₀ branched/cyclic alkoxy group, a C₃-C₂₀ branched/cyclic thioalkoxy group, a C₃-C₂₀ branched/cyclic silyl group, a C₁-C₂₀ ketone group, a C₂-C₂₀ alkoxycarbonyl group, a C₇-C₂₀ aryloxycarbonyl group, a cyano group, a carbamoyl group, a haloformyl group, a formyl group, an isocyano group, an isocyanate group, a thiocyanate group, an isothiocyanate group, a hydroxyl group, a nitro group, —CF₃, —Cl, —Br, —F, —I, a cross-linkable group, a substituted/unsubstituted aromatic or heteroaromatic group containing 5 to 60 ring atoms, an aryloxy or heteroaryloxy group containing 5 to 60 ring atoms, and any combination thereof; and R₁ is bridged to a carbon atom on an adjacent or linked aromatic ring via a bridging group, the bridging group being the same or different as a single bond, or a di-bridging group, or tri-bridging group.

17. A formulation, comprising the organic compound according to claim 1, and at least one organic solvent.

18. An organic electronic device, comprising a functional layer, the functional layer comprising the organic compound according to claim 1.

19. The organic electronic device according to claim 18, wherein the organic electronic device is selected from an organic light-emitting diode, an organic photovoltaic cell, an organic light emitting electrochemical cell, an organic field effect transistor, an organic light emitting field effect transistor, an organic laser, an organic spintronic device, an organic sensor, or an organic plasmon emitting diode.

20. The organic electronic device according to claim 18, wherein the organic electronic device is an electroluminescent device and comprises a light-emitting layer, the light-emitting layer comprises the organic compound according to claim 1.
